(19) United States
(12) Patent Application Publication
(10) Pub. No.: US 2006/0019980 A1
(43) Pub. Date: Jan. 26, 2006

(54) Methods for treating or preventing erectile dysfunction or urinary incontinence

(75) Inventors: Csaba Szabo, Gloucester, MA (US); Andrew L. Salzman, Belmont, MA (US)

Correspondence Address: WILMER CUTLER PICKERING HALE AND DORR LLP, 399 PARK AVENUE, NEW YORK, NY, 10022, US

(73) Assignee: Inotek Pharmaceutical, Corp., Beverly, MA (US)

(21) Appl. No.: 11/153,628

(22) Filed: Jun. 15, 2005

Related U.S. Application Data
(60) Provisional application No. 60/580,040, filed on Jun. 16, 2004.

(51) Int. Cl. A61K 31/473

(52) U.S. Cl. 514/284; 514/285

(57) ABSTRACT

The present invention relates to methods for treating or preventing erectile dysfunction or urinary incontinence, comprising administering to a subject in need thereof an effective amount of a compound of the invention.

This application claims the benefit of U.S. Provisional Application No. 60/580,040, filed Jun. 16, 2004, the disclosure of which is incorporated by reference herein in its entirety.

### 1. FIELD OF THE INVENTION

The present invention relates to methods for treating or preventing erectile dysfunction or urinary incontinence, comprising administering to a subject in need thereof an effective amount of a compound of the invention.

### 2. BACKGROUND OF THE INVENTION

Erectile dysfunction (“ED”) is a significant male-health issue. While estimating its prevalence is difficult, estimates range from about 15 million to 30 million sufferers worldwide.

The etiology of erectile dysfunction can be multiple, and can include mechanical trauma to the nerves (such as during prostatectomy), or it can be due to diabetes, cardiovascular diseases, induced by radiation, certain drugs, or in the elderly.

Urinary incontinence affects people of all ages and levels of physical health, both in health care settings and in the community at large. Persons suffering from urinary incontinence can be predisposed to also having urinary-tract infections, pressure ulcers, perineal rashes and urosepsis. Psychosocially, urinary incontinence can be associated with embarrassment, social stigmatization, depression and a risk of institutionalization (Herzo et al., Annu. Rev. Gerontol. Geriatr. 9:74 (1989)).

There remains, however, a need in the art for methods for treating or preventing erectile dysfunction or urinary incontinence.

Citation of any reference in Section 2 of this application is not an admission that the reference is prior art.

### 3. SUMMARY OF THE INVENTION

In one embodiment the present invention provides methods for treating or preventing erectile dysfunction or urinary incontinence, comprising administering to a subject in need thereof an effective amount of a compound having the Formula (I-149): 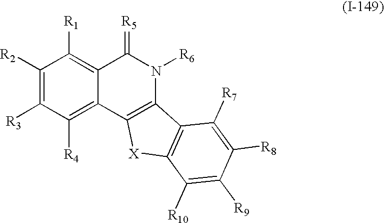
or a pharmaceutically acceptable salt thereof,
wherein:
R₅ is O, NH or S;
R₆ is —H or —C₁-C₅ alkyl;
X is —C(O)—, —CH₂—, —CH(halo)-, —CH(OH)—(CH₂)ₙ—, —CH(OH)—, —CH(-aryl)-, —O—, —NH—, —S—, —CH(NR₁₁R₁₂)— or —N(SO₂Y)—, wherein Y is —OH, —NH₂ or —(C₁-C₅ alkyl)-(3- to 7-membered monocyclic heterocycle);
R₁₁ and R₁₂ are independently -hydrogen or —C₁-C₁₀ alkyl; or N, R₁₁ and R₁₂ are taken together to form a -(nitrogen-containing 3- to 7-membered monocyclic heterocycle);
R₁ is -hydrogen, -halo, -C₁-C₁₀ alkyl, -(halo-substituted C₁-C₅ alkyl), —C₂-C₁₀ alkenyl, —(C₃-C₈ monocyclic cycloalkyl), -aryl, —NH₂, -(amino-substituted C₁-C₅ alkyl), —C(O)OH, —C(O)O(C₁-C₅ alkyl), —NO₂ or -A-B;
A is —SO₂—, —SO₂NH—, —NHC(O)—, —NHC(O)NH—, —O—, —C(O)—, —OC(O)—, —C(O)O—, —C(O)NH—, —C(O)N(C₁-C₅ alkyl)-, —NH—, —CH₂—, —S— or —C(S)—;
B is —C₁-C₁₀ alkyl, —C₂-C₁₀ alkenyl, -(3- to 7-membered monocyclic heterocycle), -(7- to 10-membered bicyclic heterocycle), —(C₃-C₈ monocyclic cycloalkyl), -aryl, —NZ₁Z₂, —(C₁-C₅ alkylene)-NZ₁Z₂, -(amino-substituted C₁-C₅ alkyl), —(C₁-C₅ alkylene)-(3- to 7membered monocyclic heterocycle), —(H₂NC(O)-substituted aryl), —C(O)OH, —C(O)O—(C₁-C₅ alkyl), —(C₁-C₅ alkylene)-C(O)OH, —C(O)O-phenyl or —C(NH)NH₂, each of which, other than —NZ₁Z₂, —C(O)OH, and —C(NH)NH₂, is unsubstituted or substituted with one or more of -(hydroxy-substituted C₁-C₅ alkyl), -(amino-substituted C₁-C₅ alkyl), —O—(C₁-C₅ alkyl), -halo, -hydroxy, —NO₂, —CN, —NZ₁Z₂, -(nitrogen-containing 3- to 7-membered monocyclic heterocycle), -(nitrogen containing 7- to 10-membered bicyclic heterocycle), —C₁-C₁₀ alkyl, —C₂-C₁₀ alkenyl, —C₂-C₁₀ alkynyl, -aryl, -benzyl, —C(O)OH, —(C₁-C₅ alkylene)-C(O)O—(C₁-C₅ alkyl), —C₁-C₅ alkylene)-OC(O)—(C₁-C₅ alkyl), or —(C₁-C₅ alkylene)-C(O)OH, each of which is unsubstituted or substituted with —C₁-C₁₀ alkyl or -(hydroxy-substituted C₁-C₅ alkyl);
R₂, R₃, R₄, R₇, R₈, R₉ and R₁₀ are independently -hydrogen, -halo, -hydroxy, —O—(C₁-C₅ alkyl), —C₁-C₁₀ alkyl, -(halo-substituted C₁-C₅ alkyl), —C₂-C₁₀ alkenyl, —(C₃-C₈ monocyclic cycloalkyl), -aryl, —NH₂, -(amino-substituted C₁-C₅ alkyl), —C(O)OH, —C(O)NH₂, —C(O)O(C₁-C₅ alkyl), —OC(O)(C₁-C₅ alkyl), —NO₂ or -A-B;
Z₁ and Z₂ are independently -hydrogen or —C₁-C₁₀ alkyl, which is unsubstituted or substituted with one or more of -halo, —OH or —NZ₃Z₄, where Z₃ and Z₄ are independently, -hydrogen or —C₁-C₅ alkyl, which is unsubstituted or substituted with one or more of -halo, -hydroxy, benzyl, or —NH₂; or N, Z₃ and Z₄ are taken together to form a -(nitrogen-containing 3- to 7-membered monocyclic heterocycle); or N, Z₁ and Z₂ are taken together to form a -(nitrogen-containing 3- to 7-membered monocyclic heterocycle); and
n is an integer ranging from 0-5.

The invention further provides methods for treating or preventing erectile dysfunction or urinary incontinence, comprising administering to a subject in need thereof an effective amount of a compound having the Formula (IV-149): 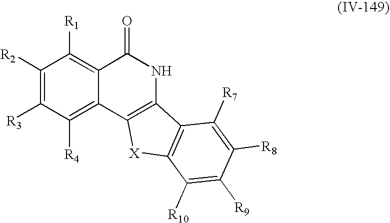
or a pharmaceutically acceptable salt thereof,
wherein:
X is —CH₂—, —O—, —NH—, or —S—;
R₁, R₂, R₃, R₄, R₇, R₈, R₉ and R₁₀ are independently -hydrogen, -halo, -hydroxy, —O—(C₁-C₅ alkyl), —C₁-C₁₀ alkyl, -(halo-substituted C₁-C₅ alkyl), —C₂-C₁₀ alkenyl, —(C₃-C₈ monocyclic cycloalkyl), -aryl, —NH₂, -(amino-substituted C₁-C₅ alkyl), —C(O)OH, —C(O)O(C₁-C₅ alkyl), —OC(O)(C₁-C₅ alkyl), —NO₂ or -A-B;
A is —SO₂—, —SO₂NH—, —NHC(O)—, —NHC(O)NH—, —O—, —CO—, —OC(O)—, —C(O)O—, —C(O)NH—, —C(O)N(C₁-C₅ alkyl)-, —NH—, —CH₂—, —S— or —C(S)—;
B is —C₁-C₁₀ alkyl, —C₂-C₁₀ alkenyl, -(3- to 7-membered monocyclic heterocycle), -(7- to 10-membered bicyclic heterocycle), —(C₃-C₈ monocyclic cycloalkyl), -aryl, —NZ₁Z₂, —(C₁-C₅ alkylene)-NZ₁Z₂, -(amino-substituted C₁-C₅ alkyl), —(C₁-C₅ alkyl)-(3- to 7-membered monocyclic heterocycle), —(H₂NC(O)-substituted aryl), —C(O)OH, —C(O)O—(C₁-C₅ alkyl), —C(O)O-phenyl or —C(NH)NH₂, each of which, other than —NZ₁Z₂, —C(O)OH, or —C(NH)NH₂, is unsubstituted or substituted with one or more of —O—(C₁-C₅ alkyl), -halo, -hydroxy, —NO₂, —CN, —NZ₁Z₂, -(nitrogen-containing 3- to 7-membered monocyclic heterocycle), —C₁-C₁₀ alkyl, —C₂-C₁₀ alkenyl, —C₂-C₁₀ alkynyl, -aryl, -benzyl, —C(O)OH, —(C₁-C₅-alkylene)-C(O)O—(C₁-C₅ alkyl) or —(C₁-C₅ alkylene)-OC(O)—(C₁-C₅ alkyl); and
Z₁ and Z₂ are independently —H or —C₁-C₁₀ alkyl, which is unsubstituted or substituted with one or more of -halo, —OH or —NZ₃Z₄, where Z₃ and Z₄ are independently, —H or —C₁-C₅-alkyl, which is unsubstituted or substituted with one or more of -halo, -hydroxy or —NH₂; or N, Z₃ and Z₄ are taken together to form a -(nitrogen-containing 3- to 7-membered monocyclic heterocycle); or N, Z₁ and Z₂ are taken together to form a -(nitrogen-containing 3- to 7-membered monocyclic heterocycle).

The invention further provides methods for treating or preventing erectile dysfunction or urinary incontinence, comprising administering to a subject in need thereof an effective amount of a compound having the Formula (I-152) 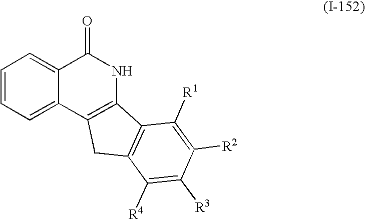
or a pharmaceutically acceptable salt thereof,
wherein
one of the R¹, R², R³ and R⁴ groups is —NH(CH₂)ₙ—N(5)(6) and the remaining groups are simultaneously —H;
R₅ and R₁ are independently —H, —C₁-C₆ alkyl or -phenyl, wherein the —C₁-C₆ alkyl or -phenyl is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, -hydroxy or —NH₂; or N, Z₃ and Z₄ are taken together to form an nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(a)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C₁-C₁₀ alkyl; or N, R⁵ and R⁶ are taken together to form a nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C₁-C₁₀ alkyl; and
n is an integer ranging from 2 to 6.

The invention further provides methods for treating or preventing erectile dysfunction or urinary incontinence, comprising administering to a subject in need thereof an effective amount of a compound having the Formula (II-152) 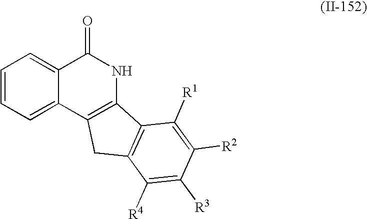
or a pharmaceutically acceptable salt thereof,
wherein
one of the R₁, R², R³ and R⁴ groups is —C(O)NH(CH₂)ₙ—N(R⁵)(R⁶) and the remaining groups are simultaneously —H;
R⁵ and R⁶ are independently —H, —C₁-C₆ alkyl or -phenyl, wherein the —C₁-C₆ alkyl or -phenyl is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, -hydroxy or —NH₂; or N, Z₃ and Z₄ are taken together to form an nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C₁-C₁₀ alkyl; or N, R⁵ and R⁶ are taken together to form a nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C₁-C₁₀ alkyl; and
n is an integer ranging from 2 to 6.

The invention further provides methods for treating or preventing erectile dysfunction or urinary incontinence, comprising administering to a subject in need thereof an effective amount of a compound having the Formula (I-153) 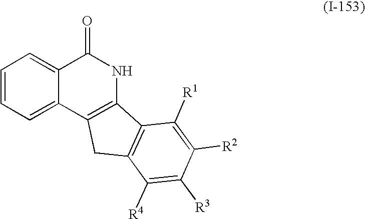
or a pharmaceutically acceptable salt thereof,
wherein
one of the R¹, R², R³ and R⁴ groups is —NHSO₂(CH₂)ₙ—N(R⁵)(R⁶) and the remaining groups are simultaneously —H;
R⁵ and R⁶ are independently —H, —C₁-C₆ alkyl or -phenyl, wherein the —C₁-C₆ alkyl or -phenyl is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, -hydroxy or —NH₂; or N, Z₃ and Z₄ are taken together to form an nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C, —C₁₀ alkyl; or N, R⁵ and R⁶ are taken together to form a nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C₁-C₁₀ alkyl; and
n is an integer ranging from 1 to 5.

The invention further provides methods for treating or preventing erectile dysfunction or urinary incontinence, comprising administering to a subject in need thereof an effective amount of a compound having the Formula (I-154) 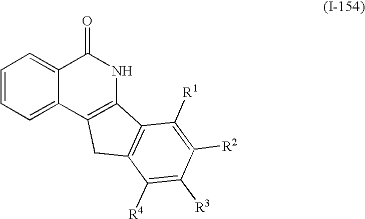
or a pharmaceutically acceptable salt thereof,
wherein
R₂ and R³ are hydrogen;
one of the R₁ and R⁴ groups is —NHC(O)—(CH₂)ₙ—NR⁵R⁶ and the remaining group is hydrogen;
R⁵ and R⁶ are independently —H, —C₁-C₆ alkyl or -phenyl, wherein the —C₁-C₆ alkyl or -phenyl is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, -hydroxy or —NH₂; or N, Z₃ and Z₄ are taken together to form an nitrogen-containing 3- to 7-membered monocyclic heterocycle which is unsubstituted or substituted with one to three of —C—C₅ alkyl, -halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C₁-C₁₀ alkyl; or N, R⁵ and R⁶ are taken together to form a nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C₁-C₁₀ alkyl; and
n is an integer ranging from 1 to 6.

The invention further provides methods for treating or preventing erectile dysfunction or urinary incontinence, comprising administering to a subject in need thereof an effective amount of a compound having the Formula (II-154) 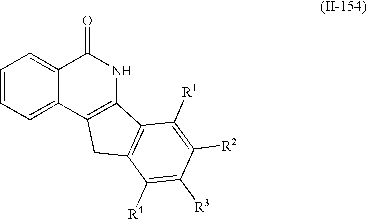
or a pharmaceutically acceptable salt thereof,
wherein
one of the R₁, R², R³, and R⁴ groups is —NHC(O)—(CH₂)ₙ—NZ₁Z₂ and the remaining groups are simultaneously hydrogen;
one of Z₁ and Z₂ is —H, —C₁-C₆ alkyl or -phenyl, and the other of Z₁ and Z₂ is -phenyl, wherein the -phenyl in each instance is unsubstituted or substituted with one or more of -halo, —OH or —N(Z₃)(Z₄), where N, Z₃ and Z₄ are taken together to form a nitrogen-containing 3- to 7-membered monocyclic heterocycle which is unsubstituted or substituted with one to three groups of —C₁-C₅ alkyl, -halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C₁-C₁₀ alkyl; or N, Z₁ and Z₂ are taken together to form a nitrogen-containing 3- to 7-membered monocyclic heterocycle, which is substituted with one to three groups of —C—C₅ alkyl, -halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C₁-C₁₀ alkyl; and
n is an integer ranging from 1 to 6.

The invention further provides methods for treating or preventing erectile dysfunction or urinary incontinence, comprising administering to a subject in need thereof an effective amount of a compound having the Formula (II-123): 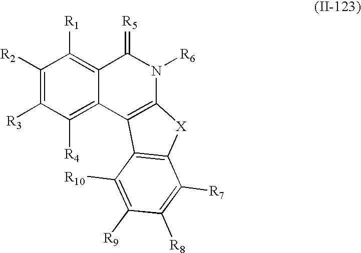
or a pharmaceutically acceptable salt thereof,
wherein:
R₅ is O, NH or S;
R₆ is —H or C₁-C₄ alkyl;
X is —C(O)—, —CH₂—, —CH(halo)-, —(C(OH)((CH₂)ₙCH₃))—, —(C(OH)(aryl))-, —O—, —NH—, —S—, —CH(NR₁₁R₁₂)— or —N(SO₂Y)—, wherein Y is —OH, —NH₂, —(C₁-C₅ alkyl)-(3- to 7-membered monocyclic heterocycle), or —(C₁-C₅ alkyl)-(7- to 10-membered bicyclic heterocycle) and n is an integer ranging from 0-5;
R₁₁ and R₁₂ are independently -hydrogen or —C₁-C₉ alkyl, or N, R₁₁ and R₁₂ are taken together to form a -(nitrogen-containing 3- to 7-membered monocyclic heterocycle), or a -(nitrogen-containing 7- to 10-membered bicyclic heterocycle);
R₁, R₂, R₃, R₄, R₇, R₉, R₉ and R₁₀ are independently -hydrogen, -halo, -hydroxy, —O—(C₁-C₅ alkyl), —C₁-C₁₀ alkyl, halo-substituted-(C₁-C₅ alkyl), —C₂-C₁₀ alkenyl, —C₃-C₈-cycloalkyl, -aryl, —NH₂, amino-substituted-(C₁-C₅ alkyl), —C(O)OH, —C(O)O(C₁-C₅ alkyl), —OC(O)(C₁-C₅ alkyl), NO₂ or -A-B;
A is —SO₂—, —SO₂NH—, —NHCO—, —NHCONH—, —O—, —CO—, —OC(O)—, —C(O)O—, —CONH—, —CON(C₁-C₄ alkyl)-, —NH—, —CH₂—, —S— or —C(S)—;
B is —C₁-C₁₀ alkyl, —C₂-C₁₀ alkenyl, -(nitrogen-containing 3- to 7-membered monocyclic heterocycle), -(nitrogen-containing 7- to 10-membered bicyclic heterocycle), -(3- to 7-membered monocyclic heterocycle), -(7- to 10-membered bicyclic heterocycle), —C₃-C₈ cycloalkyl, -aryl, —NZ₁Z₂, —(C₁-C₅ alkylene)-NZ₁Z₂, amino-substituted-(C₁-C₅ alkyl), —N(C₁-C₅ alkyl)(C₁-C₅ alkyl), —(C₁-C₅ alkyl)-(3- to 7-membered monocyclic heterocycle), or —(C₁-C₅ alkyl)-(7- to 10-membered bicyclic heterocycle), —(H₂NC(O))-substituted aryl, —(H₂NC(O))-substituted pyridyl, —C(O)OH, —C(O)O—(C₁-C₅ alkyl), —C(O)O-phenyl or —C(NH)NH₂, each of which is unsubstituted or substituted with one or more of —O—(C₁-C₅ alkyl), -halo, halo-substituted-(C₁-C₅ alkyl), HO-substituted-(C₁-C₅ alkyl), amino-substituted-(C₁-C₅ alkyl), -hydroxy, —NO₂, —NH₂, —CN, —NH(C₁-C₅ alkyl), —N(C₁-C₅ alkyl)(C₁-C₅ alkyl), -(-(nitrogen-containing 3- to 7-membered monocyclic heterocycle)), 7- to 10-membered bicycloheterocyclic amine, —C₁-C₁₀ alkyl, —C₂-C₁₀ alkenyl, —C₂-C₁₀ alkynyl, -aryl, -benzyl, —(H₂NC(O))-substituted(C₁-C₅ alkyl), carboxy-substituted-(C₁-C₅ alkyl), —C(O)OH, —C₁-C₅-alkylene-C(O)O—(C₁-C₅ alkyl) or —C₁-C₅ alkylene-OC(O)—(C₁-C₅ alkyl); and
Z₁ and Z₂ are independently —H or —C₁-C₁₀ alkyl, which is unsubstituted or substituted with one or more of -halo, —OH or —N(Z₃)(Z₄), where Z₃ and Z₄ are independently, —H or —C₁-C₅ alkyl, which is unsubstituted or substituted with one or more of -halo, -hydroxy or —NH₂; or N, Z₃ and Z₄ are taken together to form a -(nitrogen-containing 3- to 7-membered monocyclic heterocycle) or a -(nitrogen-containing 7- to 10-membered bicyclic heterocycle); or N, Z₁ and Z₂ are taken together to form a -(nitrogen-containing 3- to 7-membered monocyclic heterocycle), or a -(nitrogen-containing 7- to 10-membered bicyclic heterocycle).

The invention further provides methods for treating or preventing erectile dysfunction or urinary incontinence, comprising administering to a subject in need thereof an effective amount of a compound having the Formula (IIa-123): 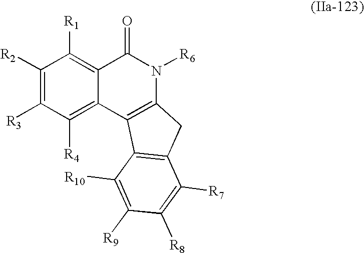
or a pharmaceutically acceptable salt thereof,
wherein:
R₆ is —H or C₁-C₄ alkyl;
R₁, R₂, R₃, R₄, R₇, R₈, R₉ and R₁₀ are independently -hydrogen, -halo, -hydroxy, —O—(C₁-C₅ alkyl), —C₁-C₁₀ alkyl, halo-substituted-(C₁-C₅ alkyl), —C₂-C₁₀ alkenyl, —C₃-C₈-cycloalkyl, -aryl, —NH₂, amino-substituted-(C₁-C₅ alkyl), —C(O)OH, —C(O)O(C₁-C₅ alkyl), —OC(O)(C₁-C₅ alkyl), NO₂ or -A-B;
A is —SO₂—, —SO₂NH—, —NHCO—, —NHCONH—, —O—, —CO—, —OC(O)—, —C(O)O—, —CONH—, —CON(C₁-C₄ alkyl)-, —NH—, —CH₂—, —S— or —C(S)—;
B is —C₁-C₁₀ alkyl, —C₂-C₁₀ alkenyl, -(nitrogen-containing 3- to 7-membered monocyclic heterocycle), -(nitrogen-containing 7- to 10-membered bicyclic heterocycle), -(3- to 7-membered monocyclic heterocycle), -(7- to 10-membered bicyclic heterocycle), —C₃-C₈ cycloalkyl, -aryl, —NZ₁Z₂, —(C₁-C₅ alkylene)-NZ₁Z₂, amino-substituted-(C₁-C₅ alkyl), —N(C₁-C₅ alkyl)(C₁-C₅ alkyl), —C₁-C₅ alkyl)-(3- to 7-membered monocyclic heterocycle), or —C₁-C₅ alkyl)-(7- to 10-membered bicyclic heterocycle), —(H₂NC(O))-substituted aryl, —(H₂NC(O))-substituted pyridyl, —C(O)OH, —C(O)O—(C₁-C₅ alkyl), —C(O)O-phenyl or —C(NH)NH₂, each of which is unsubstituted or substituted with one or more of —O—(C₁-C₅ alkyl), -halo, halo-substituted-(C₁-C₅ alkyl), HO-substituted-(C₁-C₅ alkyl), amino-substituted-(C₁-C₅ alkyl), -hydroxy, —NO₂, —NH₂, —CN, —NH(C₁-C₅ alkyl), —N(C₁-C₅ alkyl)(C₁-C₅ alkyl), -(-(nitrogen-containing 3- to 7-membered monocyclic heterocycle)), 7- to 10-membered bicycloheterocyclic amine, —C₁-C₁₀ alkyl, —C₂-C₁₀ alkenyl, —C₂-C₁₀ alkynyl, -aryl, -benzyl, —(H₂NC(O))-substituted(C₁-C₅ alkyl), carboxy-substituted-(C₁-C₅ alkyl), —C(O)OH, —C₁-C₅-alkylene-C(O)O—(C₁-C₅ alkyl) or —C₁-C₅ alkylene-OC(O)—(C₁-C₅ alkyl); and
Z₁ and Z₂ are independently —H or —C₁-C₁₀ alkyl, which is unsubstituted or substituted with one or more of -halo, —OH or —N(Z₃)(Z₄), where Z₃ and Z₄ are independently, —H or —C₁-C₅ alkyl, which is unsubstituted or substituted with one or more of -halo, -hydroxy or —NH₂; or N, Z₃ and Z₄ are taken together to form a -(nitrogen-containing 3- to 7-membered monocyclic heterocycle) or a -(nitrogen-containing 7- to 10-membered bicyclic heterocycle); or N, Z₁ and Z₂ are taken together to form a -(nitrogen-containing 3- to 7-membered monocyclic heterocycle), or a -(nitrogen-containing 7- to 10-membered bicyclic heterocycle).

The invention further provides methods for treating or preventing erectile dysfunction or urinary incontinence, comprising administering to a subject in need thereof an effective amount of a compound having the Formula (VI-123): 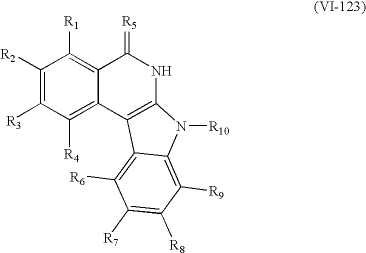
or a pharmaceutically acceptable salt thereof,
wherein:
R₅ is O. S, or NH;
R₁, R₂, R₃, R₄, R₆, R₇, R₈, and R₉ are independently -hydrogen, -halo, -hydroxy, —NH₂ NO₂, or -A-B;
A is —SO₂—, —SO₂NH—, —NHCO—, —NHCONH—, —O—, —CO—, —OC(O)—, —C(O)O—, —CONH—, —CON(C₁-C₄ alkyl)-, —NH—, —CH₂—, —S— or —C(S)—;
B is —C₁-C₁₀ alkyl, —C₂-C₁₀ alkenyl, -(nitrogen-containing 3- to 7-membered monocyclic heterocycle), -(nitrogen-containing 7- to 10-membered bicyclic heterocycle), -(3- to 7-membered monocyclic heterocycle), -(7- to 10-membered bicyclic heterocycle), —C₃-C₈ cycloalkyl, -aryl, —NZ₁Z₂, —(C₁-C₅ alkylene)-NZ₁Z₂, amino-substituted-(C₁-C₅ alkyl), —N(C₁-C₅ alkyl)(C₁-C₅ alkyl), —(C₁-C₅ alkyl)-(3- to 7-membered monocyclic heterocycle), or —C₁-C₅ alkyl)-(7- to 10-membered bicyclic heterocycle), —(H₂NC(O))-substituted aryl, —(H₂NC(O))-substituted pyridyl, —C(O)OH, —C(O)O—(C₁-C₅ alkyl), —C(O)O-phenyl or —C(NH)NH₂, each of which is unsubstituted or substituted with one or more of —O—(C₁-C₅ alkyl), -halo, halo-substituted-(C₁-C₅ alkyl), HO-substituted-(C₁-C₅ alkyl), amino-substituted-(C₁-C₅ alkyl), -hydroxy, —NO₂, —NH₂, —CN, —NH(C₁-C₅ alkyl), —N(C₁-C₅ alkyl)(C₁-C₅ alkyl), -(nitrogen-containing 3- to 7-membered monocyclic heterocycle), 7- to 10-membered bicycloheterocyclic amine, —C₁-C₁₀ alkyl, —C₂-C₁₀ alkenyl, —C₂-C₁₀ alkynyl, -aryl, -benzyl, —(H₂NC(O))-substituted(C₁-C₅ alkyl), carboxy-substituted-(C₁-C₅ alkyl), —C(O)OH, —C₁-C₅-alkylene-C(O)O—(C₁-C₅ alkyl) or —C₁-C₅ alkylene-OC(O)—(C₁-C₅ alkyl);
Z₁ and Z₂ are independently —H or —C₁-C₁₀ alkyl, which is unsubstituted or substituted with one or more of -halo, —OH or —N(Z₃)(Z₄), where Z₃ and Z₄ are independently, —H or —C₁-C₅ alkyl, which is unsubstituted or substituted with one or more of -halo, -hydroxy or —NH₂; or N, Z₃ and Z₄ are taken together to form a -(nitrogen-containing 3- to 7-membered monocyclic heterocycle) or a -(nitrogen-containing 7- to 10-membered bicyclic heterocycle); or N, Z₁ and Z₂ are taken together to form a -(nitrogen-containing 3- to 7-membered monocyclic heterocycle), or a -(nitrogen-containing 7- to 10-membered bicyclic heterocycle);
R₁₀ is —H, —C₁-C₅ alkyl, —(CH₂)ₙ—CN, —(CH₂)ₙ-aryl, —(CH₂)ₙ-(3- to 7-membered monocyclic heterocycle), —(CH₂)ₙ-(7- to 10-membered bicyclic heterocycle), —(CH₂)ₙ—COO—(C₁-C₅ alkyl), —(CH₂)ₙ—COO-aryl, —(CH₂)ₙ—COOH, —CONH—(CH₂), —COOH, —CONH—(CH₂), —COO—(C₁-C₅ alkyl), —CONH—(CH₂)ₙ-aryl, —CONHNH—(C₁-C₅ alkyl), —CONHNH-aryl, —(CH₂)ₙ—CONH₂, —(CH₂), —CONH—(C₁-C₅ alkyl), —(CH₂), —CONH-aryl, —(CH₂)ₙ—CONH—(CH₂)_{q}-aryl, —(CH₂)ₙ—CONH—(CH₂)_{q}-(3- to 7-membered monocyclic heterocycle), —(CH₂)ₙ—CONH—(CH₂)_{q}-(3- to 7-membered monocyclic heterocycle), —(CH₂)ₙ—CONH—(CH₂)_{q}—CONH₂(CH₂)ₙ—CONH—(CH₂)_{q}—CONH—(C₁-C₅ alkyl), —(CH₂)ₙ—CONH—(CH₂)_{q}—CON(C₁-C₅ alkyl)₂, —C(O)(CH₂)ₙ—(C₁-C₅ alkyl), —C(O)(CH₂)ₙ-aryl, —C(O)(CH₂), —COOH, —C(O)(CH₂)ₙ—COO—(C₁-C₅alkyl), —C(O)(CH₂)ₙ—COO-(3- to 7-membered monocyclic heterocycle), —C(O)(CH₂), —COO-(7- to 10-membered bicyclic heterocycle), —C(O)(CH₂)ₙ-phenyl, —C(O)(CH₂)ₙ-(3- to 7-membered monocyclic heterocycle), —C(O)(CH₂)ₙ-phenyl, —C(O)(CH₂)ₙ-(7- to 10-membered bicyclic heterocycle), —C(O)O(CH₂)ₙ-phenyl, —C(O)O(CH₂)ₙ-(3- to 7-membered monocyclic heterocycle), —C(O)(CH₂)ₙ-phenyl, —C(O)(CH₂)ₙ-(7- to 10-membered bicyclic heterocycle), —C(O)N((CH₂)ₙ-phenyl)₂, —C(O)N((CH₂)ₙ-phenyl)((CH₂)_{q}-3- to 7-membered monocyclic heterocycle), —C(O)N((CH₂)ₙ-phenyl)((CH₂)_{q 7}- to 10-membered bicyclic heterocycle), —C(O)N((CH₂)ₙ-(3- to 7-membered monocyclic heterocycle)₂, —C(O)N((CH₂)ₙ-7- to 10-membered bicyclic heterocycle)₂, or —SO₂NH₂;
each n is an integer ranging from 0 to 10; and
q is an integer ranging from 0 to 10.

As used herein, a compound of Formula (I-149), Formula (IV-149), Formula (I-152), Formula (II-152), Formula (I-153), Formula (I-154), Formula (II-154), Formula (II-123) Formula (IIa-123), or Formula (VI-123), or a pharmaceutically acceptable salt thereof is a “compound of the invention”

A compound of the invention is useful for treating or preventing erectile dysfunction or urinary incontinence in a subject.

The details of the invention are set forth in the accompanying description below.

### 4. DETAILED DESCRIPTION OF THE INVENTION

### 4.1 Compounds of Formula (I-149)

As stated above, the present invention encompasses methods for treating or preventing erectile dysfunction or urinary incontinence, comprising administering to a subject in need thereof an effective amount of a compound of Formula (I-149): 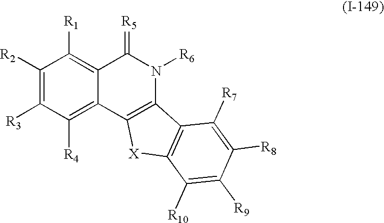
or a pharmaceutically acceptable salt thereof,
wherein X and R₁-R₁₀ are defined above for the compounds of Formula (I-149).

In one embodiment, X is —C(O)—, —CH₂—, —CH(halo)-, —CH(OH)—(CH₂)ₙ—, —CH(OH)—, —CH(-aryl)-, —O—, —NH—, —S— or —CH(NR₁₁R₁₂)—, wherein n is an integer ranging from 0-5.

In one embodiment, R₅ is O.

In another embodiment, R₅ is S.

In a further embodiment, R₅ is NH.

In another embodiment, X is —N(SO₂Y)—.

In one embodiment, A is —SO₂— or —SO₂NH—.

In another embodiment, B is —C₁-C₁₀ alkyl, —C₂-C₁₀ alkenyl, -(3- to 7-membered monocyclic heterocycle), -(7- to 10-membered bicyclic heterocycle), —(C₃-C₈ monocyclic cycloalkyl), -aryl, —NZ₁Z₂, -(amino-substituted C₁-C₅ alkyl), —(C₁-C₅ alkylene)-(-3- to 7-membered monocyclic heterocycle), —(H₂NC(O)-substituted aryl), —C(O)OH, —C(O)O—(C₁-C₅ alkyl) or —C(O)O-phenyl, each of which, other than —NZ₁Z₂, —C(O)OH, or —C(NH)NH₂, is unsubstituted or substituted with one or more of —O—(C₁-C₅ alkyl), -halo, -hydroxy, —NO₂, —NZ₁Z₂, -(nitrogen-containing 3- to 7-membered monocyclic heterocycle), —C₁-C₁₀ alkyl, —C₂-C₁₀ alkenyl, —C₂-C₁₀ alkynyl, -aryl, -benzyl, —(C₁-C₅ alkylene)-C(O)O—C₁-C₅ alkyl or —(C₁-C₅ alkylene)-OC(O)—C₁-C₅ alkyl.

In another embodiment B is -(3- to 7-membered monocyclic heterocycle), or —NZ₁Z₂, wherein -(3- to 7-membered monocyclic heterocycle) is unsubstituted or substituted with one or more of —(C₁-C₁₀ alkyl), —(C₁-C₅ alkylene)-C(O)O—(C₁-C₅ alkyl) or —(C₁-C₅ alkylene)-C(O)OH.

In another embodiment, R₁-R₄ are hydrogen.

In a further embodiment, at least one of R₁, R₂, R₃, R₄, R₇, R, R₉, and R₁₀ is other than hydrogen.

In other illustrative embodiments R₅ and X in the compounds of Formula (I-149) are as set forth below:

| R⁵ | X |
|---|---|
| NH | —C(O)— |
| NH | —CH₂— |
| NH | —CH(halo)- |
| NH | —CH(OH)CH₂)ₙ— |
| NH | —CH(OH)— |
| NH | —CH(-aryl)- |
| NH | —O— |
| NH | —NH— |
| NH | —S— |
| NH | —CH(NR¹¹R¹²)— |
| NH | —N(SO₂Y)— |
| S | —C(O)— |
| S | —CH₂— |
| S | —CH(halo)- |
| S | —CH(OH)(CH₂)ₙ— |
| S | —CH(OH)— |
| S | —CH(-aryl)- |
| S | —O— |
| S | —NH— |
| S | —S— |
| S | —CH(NR¹¹R¹²)— |
| S | —N(SO₂Y)— |
| O | —C(O)— |
| O | —CH₂— |
| O | —CH(halo)- |
| O | —CH(OH)(CH₂)ₙ— |
| O | —CH(OH)— |
| O | —CH(-aryl)- |
| O | —O— |
| O | —NH— |
| O | —S— |
| O | —CH(NR¹¹R¹²)— |
| O | —N(SO₂Y)— |

pharmaceutically acceptable salts thereof.

In another embodiment, the compounds of Formula (I-149) have the Formula (8-149): 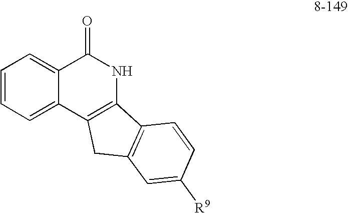
and pharmaceutically acceptable salts thereof,
wherein:
R₉ is -hydrogen or -A-B;
A is —SO₂—, —SO₂NH— or —NHC(O)—;
B is —C₁-C₁₀ alkyl, -(3- to 7-membered monocyclic heterocycle), -(7- to 10-membered bicyclic heterocycle), —NZ₁Z₂, —(C₁-C₅ alkylene)-NZ₁Z₂, —C₁-C₅ alkylene)-(3- to 7-membered monocyclic heterocycle), or —C(NH)NH₂, each of which, other than —NZ₁Z₂, and —C(NH)NH₂, is unsubstituted or substituted with one or more of —CN, —NZ₁Z₂, -(nitrogen-containing 3- to 7-membered monocyclic heterocycle), —C₁-C₁₀ alkyl, -aryl, -benzyl, —(C₁-C₅ alkylene)-C(O)O—(C₁-C₅ alkyl), or —(C₁-C₅ alkylene)-OC(O)—(C₁-C₅ alkyl); and
Z₁ and Z₂ are independently -hydrogen or —C₁-C₈ alkyl, which is unsubstituted or substituted with one or more of -hydroxy, or —NZ₃Z₄, where Z₃ and Z₄ are independently —H or —C₁-C₅ alkyl, which is unsubstituted or substituted with one or more of -hydroxy, -benzyl, or —NH₂; or N, Z₃ and Z₄ are taken together to form a -(nitrogen-containing 3- to 7-membered monocyclic heterocycle); or N, Z₁ and Z₂ are taken together to form a -(nitrogen-containing 3- to 7-membered monocyclic heterocycle).

Illustrative examples of compounds of Formula (8-149) are set forth below:

| Compound | R⁹ |
|---|---|
| 8a-149 | —SO₂-(4-Methyl-piperazin-1-yl) |
| 8b-149 | —SO₂-(4-CH₂CO₂Me-piperazin-1-yl) |
| 8c-149 | —SO₂-(4-CH₂CO₂H-piperazin-1-yl) |
| 8d-149 | —SO₂-(imidazol-1-yl) |
| 8e-149 | —SO₂-(prolinol) |
| 8f-149 | —SO₂-(morpholin-4-yl) |
| 8g-149 | —SO₂NHCH₂CH₂NMe₂ |
| 8h-149 | —SO₂NHCH₂CH₂-(piperidin-1-yl) |
| 8i-149 | —SO₂NHCH₂CH₂N-(pyridin-2-yl) |
| 8j-149 | —SO₂NHCH₂CH₂-(morpholin-4-yl) |
| 8k-149 | —SO₂NHCH₂CH₂-(2-N-Me-(tetrahydropyrrolidin-1-yl)) |
| 8l-149 | —SO₂NHCH₂CH₂CH₂-(morpholin-4-yl) |
| 8m-149 | —SO₂NHCH₂CH₂CH₂-(tetrahydropyrrolidin-1-yl) |
| 8n-149 | —SO₂NHCH₂CH₂CH₂-(imidazol-1-yl) |
| 8o-149 | —SO₂NHCH₂CH₂CH₂-(4-methylpiperazin-1-yl) |
| 8p-149 | —SO₂N(CH₂CH₂NEt₂)₂ |
| 8q-149 | —SO₂—N(CH₂CH₂NMe₂)₂ |
| 8r-149 | —SO₂N(CH₂CH₂OH)₂ |
| 8s-149 | —SO₂NHCH₂CH₂CN |
| 8t-149 | —SO₂NHC(NH)NH₂ |
| 8u-149 | —SO₂NH[4-(1,2,4-triazole)] |
| 8v-149 | —SO₂NH[4-(morpholin-4-yl)phenyl] |
| 8w-149 | —SO₂NHCH₂CH₂(4-N-benzylpiperidine) |
| 8x-149 | —SO₂NHCH₂CH₂(2-thienyl) |
| 8y-149 | —SO₂NH[1-(4-azabenzimidazole)] |
| 8z-149 | —SO₂NH[1-(4-(2′-pyridyl)piperazine)] |
| 8aa-149 | —SO₂NHCH₂CH₂N[CH₂CH₂OH]₂ |
| 8ab-149 | —SO₂NH[1-(4-benzylpiperazine)] |
| 8ac-149 | —SO₂NH₂ |
| 8ad-149 | —SO₂NHCH₂CH₂Ph |
| 8ae-149 | —SO₂NHCH₂CH₂[4-OMe-(phenyl)] |
| 8af-149 | —SO₂NHC(O)(morpholin-4-yl) |

and pharmaceutically acceptable salts thereof.

In one embodiment the above illustrative examples are in the form of their camphorsulphonic acid salt.

In another embodiment, the compounds of Formula (I-149) have the Formula 13-149: 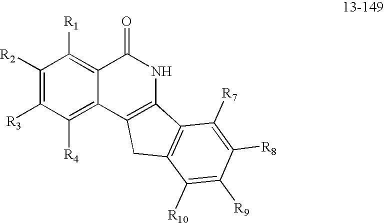
and pharmaceutically acceptable salts thereof,
wherein:
R₁, R₂, R₃, R₄, R₇, R₈, R₉ and R₁₀ are defined as above for Formula (I-149)

In one embodiment, R₉ is -A-B, wherein -A- is —SO₂— or —SO₂NH—.

In another embodiment, R₁-R₄ are each hydrogen.

In a further embodiment, at least one of R₁, R₂, R₃, R₄, R₇, R₈, R₉ and R₁₀ is other than hydrogen.

In one embodiment, A is other than CONH—.

In another embodiment, the compounds of Formula (I-149) have the Formula 22-149: 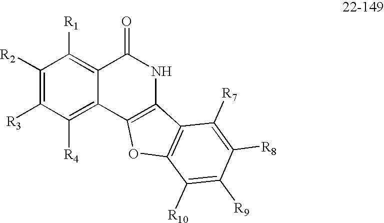
and pharmaceutically acceptable salts thereof,
wherein:
R₁-R₄ and R₇-R₁₀ are as defined above for Formula (I-149).

In one embodiment, R₉ is -A-B, wherein -A- is —SO₂— or —SO₂NH—.

In another embodiment, R₁-R₄ are each hydrogen.

In a further embodiment, at least one of R₁, R₂, R₃, R₄, R₇, R₈, R₉ and R₁₀ is other than hydrogen.

In another embodiment, the compounds of Formula (I-149) have the Formula 37-149: 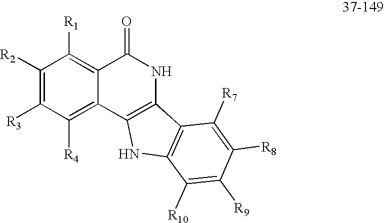
and pharmaceutically acceptable salts thereof,
wherein:
R₁-R₄ and R₇-R₁₀ are as defined above for Formula (I-149).

In one embodiment, R₁-R₄ are each hydrogen.

In another embodiment, R₉ is -A-B, wherein -A- is —SO₂— or —SO₂NH—.

In a further embodiment, at least one of R₁, R₂, R₃, R₄, R₇, R₈, R₉ and R₁₀ is other than hydrogen.

In another embodiment, the compounds of Formula (I-149) have the Formula 40-149: 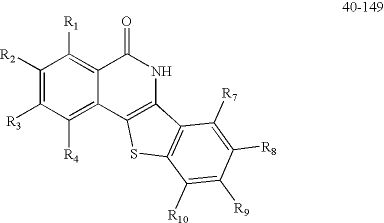
and pharmaceutically acceptable salts thereof,
wherein:
R₁-R₄ and R₇-R₁₀ are as defined above for Formula (I-149).

In one embodiment, R₁-R₄ are each hydrogen.

In one embodiment, R₉ is -A-B, wherein -A- is —SO₂— or —SO₂NH—.

In a further embodiment, at least one of R₁, R₂, R₃, R₄, R₇, R₈, R₉ and R₁₀ is other than hydrogen.

In another embodiment, the compounds of Formula (I-149) have the Formula (Ia-149): 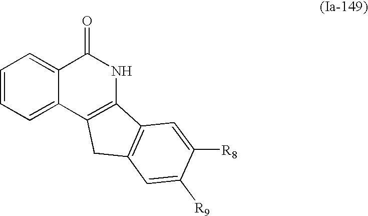
and pharmaceutically acceptable salts thereof,
wherein R₈ and R₉ are as defined above for the compounds of Formula (I-149).

In one embodiment, the compounds of Formula (Ia-149) are those wherein R₈ is —H, R₉ is -A-B, A is —SO₂— and B is —NZ₁Z₂ or —(C₁-C₅ alkylene)-NZ₁Z₂.

Illustrative examples of compounds of Formula (Ia-149) are set forth below:

| Com- | | |
|---|---|---|
| pound | R₈ | R₉ |
| 43-149 | —H | —NHC(O)CH₂N(CH₃)₂ |
| 45-149 | —NHC(O)CH₂N(CH₃)₂ | —H |
| 46-149 | —SO₂NH(CH₂)₃- | —H |
| | (morpholin-4-yl) | |
| 47-149 | —H | —NHC(O)(CH₂)₃-(morpholin-4-yl) |
| 53b-149 | —NO₂ | —H |
| 53a-149 | —H | —NO₂ |
| 99-149 | —F | —H |
| 100-149 | —H | —F |
| 54b-149 | —NH₂ | —H |
| 54a-149 | —H | —NH₂ |
| 103-149 | —H | —NHCOCH₂OAc |
| 104-149 | —H | —NHCOCH₂OH |
| 105-149 | —H | —NHCONH-n-propyl |
| 106-149 | —H | —SO₂NH(CH₂)₃-phenyl |
| 107-149 | —F | —SO₂NH(CH₂)₃-(morpholin-4-yl) |
| 108-149 | —F | —SO₂NH-(morpholin-4-yl) |
| 109-149 | —F | —SO₂-imidazole |
| 110-149 | —H | —SO₃Na |
| 111-149 | —SO₃Na | —H |

and pharmaceutically acceptable salts thereof.

In one embodiment the above illustrative examples are in the form of their camphorsulphonic acid salt.

In another embodiment, the compounds of Formula (I-149) have the Formula (Ib-149): 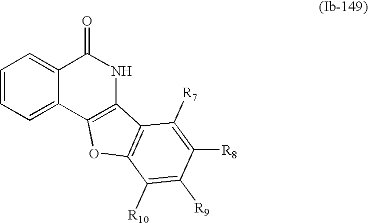
and pharmaceutically acceptable salts thereof,
wherein R₇, R₈, R₉ and R₁₀ are as defined above for the compounds of Formula (I-149).

Illustrative examples of compounds of Formula (Ib-149) are set forth below:

| Com- | | | | |
|---|---|---|---|---|
| pound | R₇ | R₈ | R₉ | R₁₀ |
| 22a-149 | —H | —H | —H | —H |
| 22b-149 | —H | —OMe | —H | —H |
| 22c-149 | —H | —H | —OMe | —H |
| 22d-149 | —H | —H | —H | —OMe |
| 22e-149 | —H | —Me | —H | —H |
| 22f-149 | —H | —COOH | —H | —H |
| 22g-149 | —H | —H | —COOH | —H |
| 23a-149 | —H | —OH | —H | —H |
| 23b-149 | —H | —H | —OH | —H |
| 23c-149 | —H | —H | —H | —OH |
| 25a-149 | —H | —H | —(CH₂)₄OH | —H |
| 25b-149 | —H | —H | —(CH₂)₅OH | —H |
| 25c-149 | —H | —H | —(CH₂)₆OH | —H |
| 25d-149 | —H | —H | —(CH₂)₄COOH | —H |
| 25e-149 | —H | —H | —(CH₂)₅COOH | —H |
| 26a-149 | —H | —C(O)NH(CH₂)₃- | —H | —H |
| | | (morpholin-4-yl) | | |
| 26b-149 | —H | —C(O)NH(CH₂)₂—COOH | —H | —H |
| 26c-149 | —H | —C(O)NH(CH₂)₃—N-(1,3- | —H | —H |
| | | imidazole) | | |
| 26d-149 | —H | —C(O)NH(CH₂)₂—NMe₂ | —H | —H |

and pharmaceutically acceptable salts thereof.

In another embodiment, the compounds of Formula (I-149) have the Formula (Ic-149): 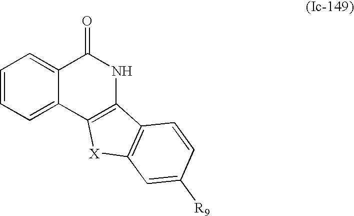
and pharmaceutically acceptable salts thereof,
where X and R₉ are as defined above for Formula (I-149).

Illustrative examples of compounds of Formula (Ic-149) are set forth below:

| Compound | X | R₉ |
|---|---|---|
| 34-149 | —N(SO₃H)— | —SO₃H |
| 35a-149 | —N(SO₂NH₂)— | —SO₂NH₂ |
| 35b-149 | —N[SO₂NH(CH₂)₃- | —SO₂NH(CH₂)₃- |
| | (morpholin-4-yl)]- | (morpholin-4-yl) |
| 40a-149 | —S— | —H |

and pharmaceutically acceptable salts thereof.

In another embodiment, the compounds of Formula (I-149) have the Formula (Id-149): 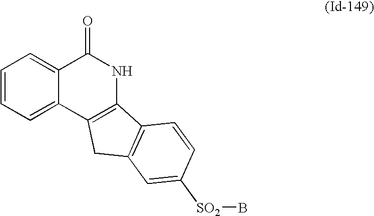
and pharmaceutically acceptable salts thereof,
where B is as defined above for the compounds of Formula (I-149).

In one embodiment, B is —NZ₁Z₂ or —(C₁-C₅ alkylene)-NZ₁Z₂.

### 4.2 Compounds of Formula (II-149)

In another embodiment, the compounds of Formula (I-149) have the Formula (II-149): 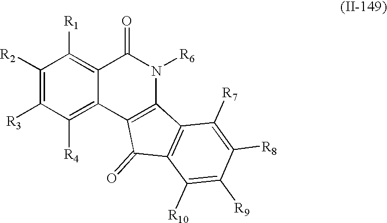
and pharmaceutically acceptable salts thereof,
wherein:
R₁ is -hydrogen, -halo, —C₁-C₁₀ alkyl, -(halo-substituted C₁-C₅ alkyl), —C₂-C₁₀ alkenyl, —(C₃-C₈ monocyclic cycloalkyl), -aryl, —NH₂, -(amino-substituted C₁-C₅ alkyl), —C(O)OH, —C(O)O(C₁-C₅ alkyl), —NO₂ or -A′-B′;
A′ is —SO₂—, —SO₂NH—, —NHC(O)—, —NHC(O)NH—, —C(O)—, —C(O)O—, —C(O)NH—, —C(O)N(C₁-C₅ alkyl)-, —NH—, —CH₂—, —S— or —C(S)—;
B′ is —C₁-C₁₀ alkyl, —C₂-C₁₀ alkenyl, -(3- to 7-membered monocyclic heterocycle), -(7- to 10-membered bicyclic heterocycle), —(C₃-C₈ monocyclic cycloalkyl), -aryl, -(amino-substituted C₁-C₅ alkyl), —(C₁-C₅ alkylene)-(3- to 7-membered monocyclic heterocycle), —(H₂NC(O)-substituted aryl), —C(O)OH, —C(O)O—(C₁-C₅ alkyl), —(C₁-C₅ alkylene)-C(O)OH, —C(O)O-phenyl or —NZ₁Z₂; and
R₂, R₃, R₄, R₆, R₇, R₈, R₉, and R₁₀ are as defined above for the compounds of Formula (I-149).

In one embodiment, B′ is a -(nitrogen-containing 3- to 7-membered monocyclic heterocycle).

In a further embodiment, at least one of R₁, R₂, R₃, R₄, R₇, R₈, R₉ and R₁₀ is not hydrogen.

In another embodiment, at least one of R₂, R₄ and R₁₀ is other than hydrogen.

### 4.3 Compounds of Formula (III-149)

In another embodiment the compounds of Formula (I-149) have the Formula (III-149): 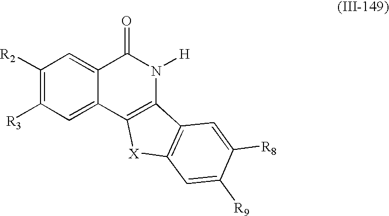
and pharmaceutically acceptable salts thereof,
wherein:
X is —CH₂— or —O—;
R₂ and R₃ are independently -hydrogen, -halo, -hydroxy, -(halo-substituted C₁-C₅ alkyl), —O—(C₁-C₅ alkyl), —C₁-C₅ alkyl, —NO₂, —NH₂, —C(O)NH₂, —C(O)OH, —OC(O)—(C₁-C₅ alkyl), or —C(O)O—(C₁-C₅ alkyl);
R₉ and R₉ are independently -hydrogen or -A-B;
A is —SO₂—, —SO₂NH— or —NHC(O)—;
B is —C₁-C₅ alkyl, —NZ₁Z₂, -(3- to 7-membered monocyclic heterocycle), or -(7- to 10-membered bicyclic heterocycle), each of which, other than —NZ₁Z₂, is unsubstituted or substituted with one or more of -(hydroxy-substituted C₁-C₅ alkyl), -(amino-substituted C₁-C₅ alkyl), -(nitrogen-containing 3- to 7-membered monocyclic heterocycle), or -(nitrogen-containing 7- to 10-membered bicyclic heterocycle), each of which is unsubstituted or substituted with —C₁-C₁₀ alkyl, or -(hydroxy-substituted C₁-C₅ alkyl); and Z₁ and Z₂ are independently -hydrogen or —C₁-C₈ alkyl, which is unsubstituted or substituted with one or more of -hydroxy or —NZ₃Z₄, where Z₃ and Z₄ are independently —H or —C₁-C₅ alkyl, which is unsubstituted or substituted with one or more of -hydroxy or —NH₂; or N, Z₃ and Z₄ are taken together to form a -(nitrogen-containing 3- to 7-membered monocyclic heterocycle); or N, Z₁ and Z₂ are taken together to form a -(nitrogen-containing 3- to 7-membered monocyclic heterocycle).

In one embodiment, —X— is —C₁H₂—.

In another embodiment, —X— is —O—.

In one embodiment, R₈ is hydrogen and R₉ is -A-B.

In another embodiment, R₈ is -A-B and R₉ is hydrogen.

In one embodiment, either R₈ is hydrogen and R₉ is -A-B, or R₈ is -A-B and R₉ is hydrogen.

In still another embodiment, R₂, R₃ and R₈ are hydrogen and R₉ is -A-B, wherein A is —SO₂— or —SO₂NH—.

In a further embodiment, at least one of R₂, R₃, R₈ and R₉ is not hydrogen.

### 4.4 Compounds of Formula (Iv-149)

The present invention further provides methods for treating or preventing erectile dysfunction or urinary incontinence, comprising administering to a subject in need thereof an effective amount of a compound of Formula (IV-149): 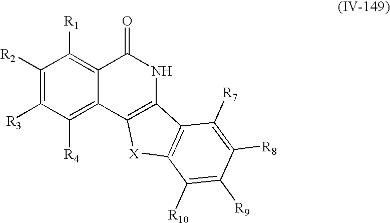
or a pharmaceutically acceptable salt thereof,
wherein:
X, R₁, R₂, R₃, R₄, R₇, R₈, R₉, and R₁₀ are as defined above for the compounds of Formula (IV-149).

In one embodiment, R₉ is -A-B, wherein -A- is —SO₂— or —SO₂NH—.

In another embodiment, R₁-R₄ are hydrogen.

In a further embodiment, at least one of R₁, R₂, R₃, R₄, R₇, R₈, R₉ and R₁₀ is other than hydrogen.

In one embodiment, X is —CH₂— and R₉ is -A-B, wherein -A- is —SO₂— or —SO₂NH—.

In another embodiment, X is —CH₂— and R₁-R₄ are hydrogen.

In a further embodiment, X is CH₂— and at least one of R₁, R₂, R₃, R₄, R₇, R₈, R₉ and R₁₀ is other than hydrogen.

In one embodiment, X is —O— and R₁-R₄ are hydrogen.

In another embodiment, X is —O— and R₉ is -A-B, wherein -A- is —SO₂— or —SO₂NH—.

In a further embodiment, X is —O— and at least one of R₁, R₂, R₃, R₄, R₇, R₈, R₉ and R₁₀ is other than hydrogen.

In one embodiment, X is —NH— and R₁-R₄ are hydrogen.

In one embodiment, X is —NH— and R₉ is -A-B, wherein -A- is —SO₂— or —SO₂NH—.

In a further embodiment, X is —NH— and at least one of R₁, R₂, R₃, R₄, R₇, R₈, R₉, and
R₁₀ is other than hydrogen.

In one embodiment, X is —S— and R₁-R₄ are hydrogen.

In one embodiment, X is —S— and R₉ is -A-B, wherein -A- is —SO₂—, or —SO₂NH—.

In a further embodiment, X is —S— and at least one of R₁, R₂, R₃, R₄, R₇, R₈, R₉, and
R₁₀ is other than hydrogen.

### 4.5 Compounds of Formula (I-152)

The present invention provides methods for treating or preventing erectile dysfunction or urinary incontinence, comprising administering to a subject in need thereof an effective amount of a compound according to Formula (I-152), below: 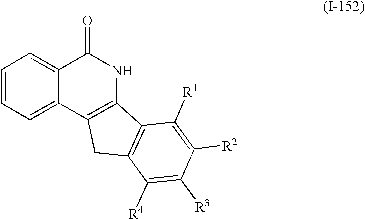
or a pharmaceutically acceptable salt thereof,
wherein:
R¹, R², R³ and R⁴ are as defined above for the compounds of Formula (I-152).

In one embodiment, R¹ is —NH(CH₂), —N(R⁵)(R⁶) and R², R³ and R⁴ are each hydrogen.

In another embodiment, R² is —NH(CH₂)ₙ—N(R⁵)(R⁶) and R₁, R³ and R⁴ are each hydrogen.

In another embodiment, R³ is —NH(CH₂), —N(R⁵)(R⁶) and R₁, R² and R⁴ are each hydrogen.

In still another embodiment, R⁴ is —NH(CH₂)ₙ—N(R⁵)(R⁶) and R₁, R² and R³ are each hydrogen.

In another embodiment, n is 2.

In still another embodiment, n is 3.

In yet another embodiment, n is 4.

In a further embodiment, n is 5.

In another embodiment, n is 6.

In various embodiments, —N(R⁵)(R⁶) is:

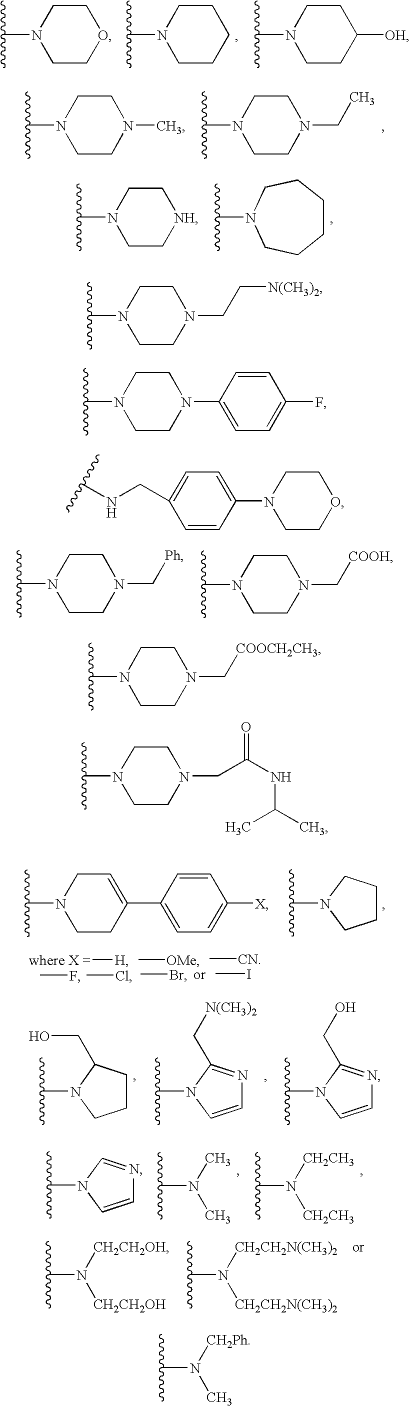

Illustrative examples of the compounds of Formula (I-152) include the compounds of Formula (Ia-152) as set forth below:

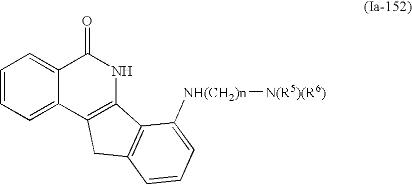

and pharmaceutically acceptable salts thereof,

| Compound | n | —N(R⁵)(R⁶) |
|---|---|---|
| 1a-152 | 2 | —N(CH₃)₂ |
| 1b-152 | 3 | —N(CH₃)₂ |
| 1c-152 | 4 | —N(CH₃)₂ |
| 1d-152 | 5 | —N(CH₃)₂ |
| 1e-152 | 6 | —N(CH₃)₂ |
| 2a-152 | 2 | 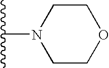 |
| 2b-152 | 3 | 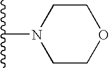 |
| 2c-152 | 4 | 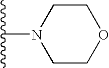 |
| 2d-152 | 5 | 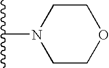 |
| 2e-152 | 6 | 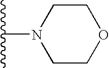 |

and pharmaceutically acceptable salts thereof.

Other illustrative examples of the compounds of Formula (I-152) include the compounds of Formula (Ib-152) as set forth below:

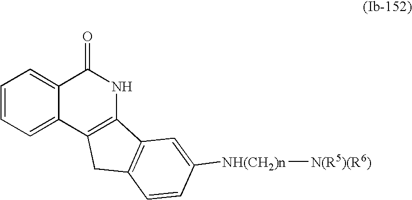

and pharmaceutically acceptable salts thereof,

| Compound | n | —N(R⁵)(R⁶) |
|---|---|---|
| 3a-152 | 2 | —N(CH₃)₂ |
| 3c-152 | 3 | —N(CH₃)₂ |
| 3c-152 | 4 | —N(CH₃)₂ |
| 3d-152 | 5 | —N(CH₃)₂ |
| 3e-152 | 6 | —N(CH₃)₂ |
| 4a-152 | 2 | 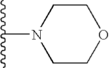 |
| 4b-152 | 3 | 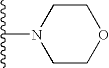 |
| 4c-152 | 4 | 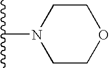 |
| 4d-152 | 5 | 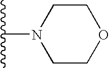 |
| 4e-152 | 6 | 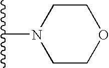 |

and pharmaceutically acceptable salts thereof.

Other illustrative examples of the compounds of Formula (I-152) include the compounds of Formula (Ic-152) as set forth below:

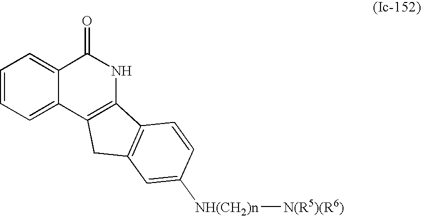

and pharmaceutically acceptable salts thereof,

| Compound | n | —N(R⁵)(R⁶) |
|---|---|---|
| 5a-152 | 2 | —N(CH₃)₂ |
| 5b-152 | 3 | —N(CH₃)₂ |
| 5c-152 | 4 | —N(CH₃)₂ |
| 5d-152 | 5 | —N(CH₃)₂ |
| 5e-152 | 6 | —N(CH₃)₂ |
| 6a-152 | 2 | 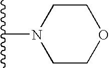 |
| 6b-152 | 3 | 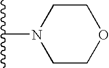 |
| 6c-152 | 4 | 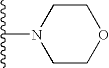 |
| 6d-152 | 5 | 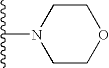 |
| 6e-152 | 6 | 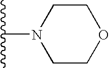 |

and pharmaceutically acceptable salts thereof.

Other illustrative examples of the compounds of Formula (I-152) include the compounds of Formula (Id-152) as set forth below:

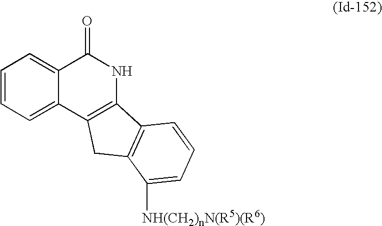

and pharmaceutically acceptable salts thereof,

| Compound | n | —N(R⁵)(R⁶) |
|---|---|---|
| 7a-152 | 2 | —N(CH₃)₂ |
| 7b-152 | 3 | —N(CH₃)₂ |
| 7c-152 | 4 | —N(CH₃)₂ |
| 7d-152 | 5 | —N(CH₃)₂ |
| 7e-152 | 6 | —N(CH₃)₂ |
| 8a-152 | 2 | 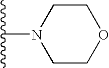 |
| 8b-152 | 3 | 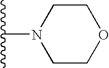 |
| 8c-152 | 4 | 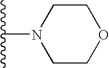 |
| 8d-152 | 5 | 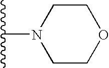 |
| 8e-152 | 6 | 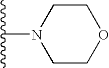 |

and pharmaceutically acceptable salts thereof.

### 4.6 Compounds of Formula (II-152)

The present invention provides methods for treating or preventing erectile dysfunction or urinary incontinence, comprising administering to a subject in need thereof an effective amount of a compound according to Formula (II-152), below: 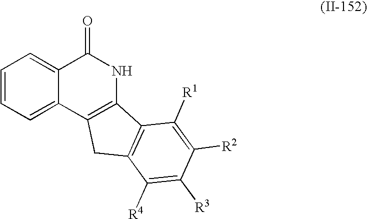
or a pharmaceutically acceptable salt thereof,
wherein:
R¹, R², R³ and R⁴ are as defined above for the compounds of Formula (II-152).

In one embodiment, R¹ is —C(O)NH—(CH₂)ₙ—N(R⁵)(R⁶) and R², R³ and R⁴ are each hydrogen.

In another embodiment, R² is —C(O)NH—(CH₂)ₙ—N(R⁵)(R⁶) and R₁, R³ and R⁴ are each hydrogen.

In another embodiment, R³ is —C(O)NH—(CH₂)ₙ—N(R⁵)(R⁶) and R₁, R² and R⁴ are each hydrogen.

In still another embodiment, R⁴ is —C(O)NH—(CH₂)ₙ—N(R⁵)(R⁶) and R₁, R² and R³ are each hydrogen.

In another embodiment, n is 2.

In still another embodiment, n is 3.

In yet another embodiment, n is 4.

In a further embodiment, n is 5.

In various embodiments, —N(R⁵)(R⁶) is:

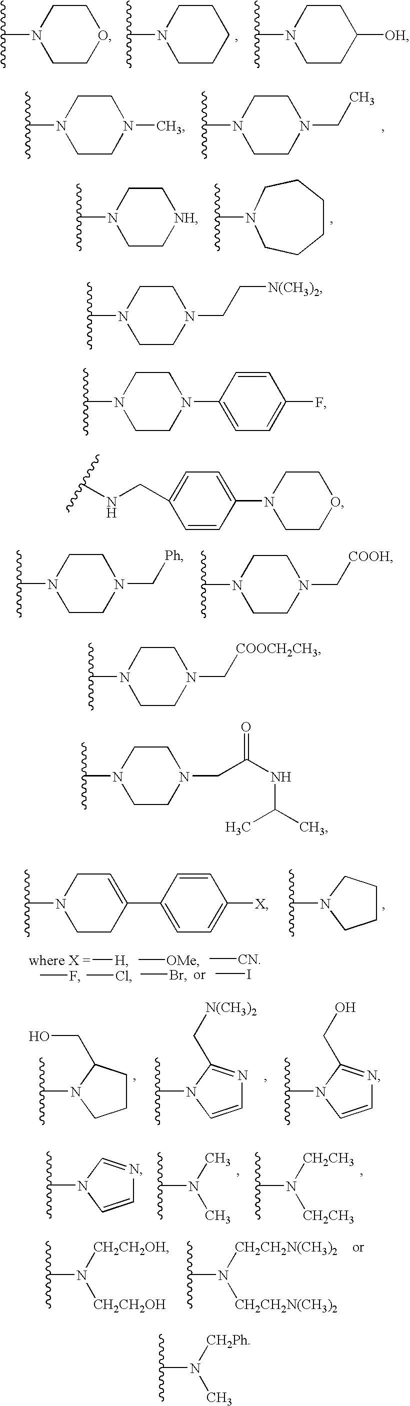

Illustrative examples of the compounds of Formula (II-152) include the compounds of Formula (IIa-152) as set forth below:

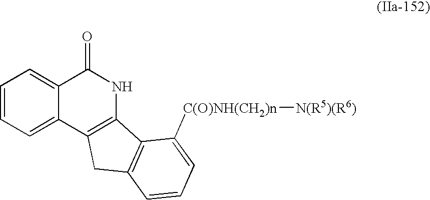

and pharmaceutically acceptable salts thereof,

| Compound | n | —N(R⁵)(R⁶) |
|---|---|---|
| 9a-152 | 2 | —N(CH₃)₂ |
| 9b-152 | 3 | —N(CH₃)₂ |
| 9c-152 | 4 | —N(CH₃)₂ |
| 9d-152 | 5 | —N(CH₃)₂ |
| 10a-152 | 2 | 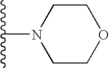 |
| 10b-152 | 3 | 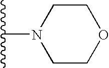 |
| 10c-152 | 4 | 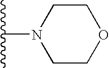 |
| 10d-152 | 5 | 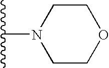 |

and pharmaceutically acceptable salts thereof.

Other illustrative examples of the compounds of Formula (II-152) include the compounds of Formula (IIb-152) as set forth below:

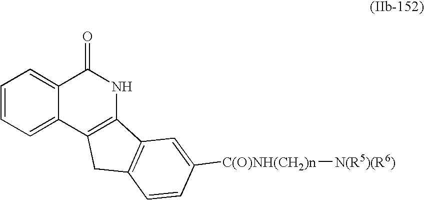

and pharmaceutically acceptable salts thereof,

| Compound | n | —N(R⁵)(R⁶) |
|---|---|---|
| 11a-152 | 2 | —N(CH₃)₂ |
| 11b-152 | 3 | —N(CH₃)₂ |
| 11c-152 | 4 | —N(CH₃)₂ |
| 11d-152 | 5 | —N(CH₃)₂ |
| 12a-152 | 2 | 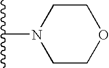 |
| 12b-152 | 3 | 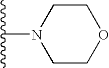 |
| 12c-152 | 4 | 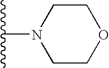 |
| 12d-152 | 5 | 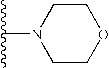 |

and pharmaceutically acceptable salts thereof.

Other illustrative examples of the compounds of Formula (II-152) include the compounds of Formula (IIc-152) as set forth below:

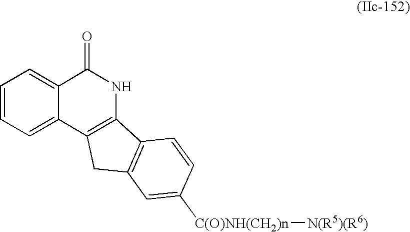

and pharmaceutically acceptable salts thereof,

| Compound | n | —N(R⁵)(R⁶) |
|---|---|---|
| 13a-152 | 2 | —N(CH₃)₂ |
| 13b-152 | 3 | —N(CH₃)₂ |
| 13c-152 | 4 | —N(CH₃)₂ |
| 13d-152 | 5 | —N(CH₃)₂ |
| 14a-152 | 2 | 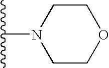 |
| 14b-152 | 3 | 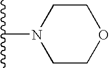 |
| 14c-152 | 4 | 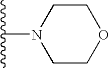 |
| 14d-152 | 5 | 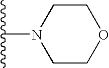 |

and pharmaceutically acceptable salts thereof.

Other illustrative examples of the compounds of Formula (II-152) include the compounds of Formula (IId-152) as set forth below:

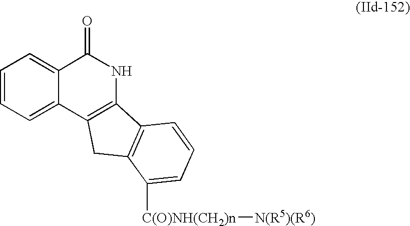

and pharmaceutically acceptable salts thereof,

| Compound | n | —N(R⁵)(R⁶) |
|---|---|---|
| 15a-152 | 2 | —N(CH₃)₂ |
| 15b-152 | 3 | —N(CH₃)₂ |
| 15c-152 | 4 | —N(CH₃)₂ |
| 15d-152 | 5 | —N(CH₃)₂ |
| 16a-152 | 2 | 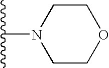 |
| 16b-152 | 3 | 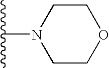 |
| 16c-152 | 4 | 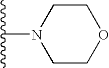 |
| 16d-152 | 5 | 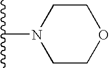 |

and pharmaceutically acceptable salts thereof.

### 4.7 Compounds of Formula (I-153)

The present invention provides methods for treating or preventing erectile dysfunction or urinary incontinence, comprising administering to a subject in need thereof an effective amount of a compound according to Formula (I-153), below: 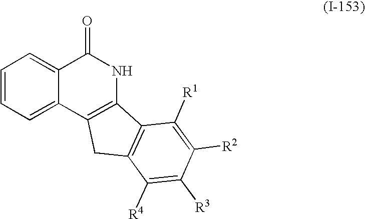
or a pharmaceutically acceptable salt thereof,
wherein:
R¹, R², R³ and R⁴ are as defined above for the compounds of Formula (I-153).

In one embodiment, R¹ is —NHSO₂—(CH₂)ₙ—N(R⁵)(R⁶) and R², R³ and R⁴ are each hydrogen.

In another embodiment, R² is —NHSO₂—(CH₂)ₙ—N(R⁵)(R⁶) and R¹, R³ and R⁴ are each hydrogen.

In another embodiment, R³ is —NHSO₂—(CH₂)ₙ—N(R⁵)(R₆) and R₁, R₂ and R⁴ are each hydrogen.

In still another embodiment, R⁴ is —NHSO₂—(CH₂), —N(R⁵)(R⁶) and R₁, R² and R³ are each hydrogen.

In one embodiment, n is 1.

In another embodiment, n is 2.

In still another embodiment, n is 3.

In yet another embodiment, n is 4.

In a further embodiment, n is 5.

In various embodiments, —N(R⁵)(R⁶) is:

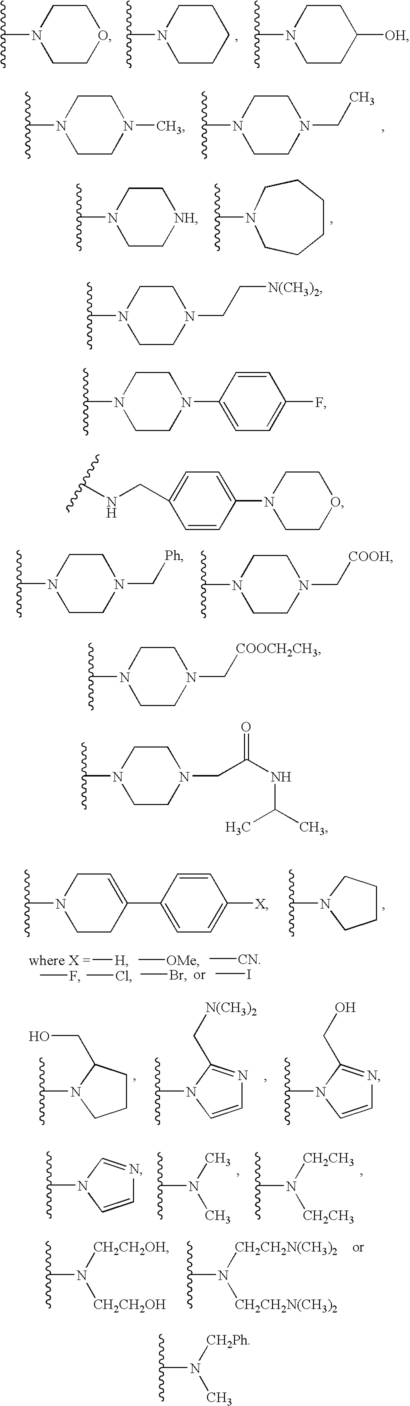

Illustrative examples of the compounds of Formula (I-153) include the compounds of Formula (Ia-153) as set forth below:

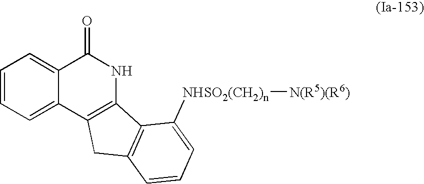

and pharmaceutically acceptable salts thereof,

| Compound | n | —N(R⁵)(R⁶) |
|---|---|---|
| 1a-153 | 1 | —N(CH₃)₂ |
| 1b-153 | 2 | —N(CH₃)₂ |
| 1c-153 | 3 | —N(CH₃)₂ |
| 1d-153 | 4 | —N(CH₃)₂ |
| 1e-153 | 5 | —N(CH₃)₂ |
| 2a-153 | 1 | 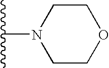 |
| 2b-153 | 2 | 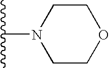 |
| 2c-153 | 3 | 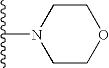 |
| 2d-153 | 4 | 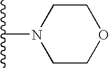 |
| 2e-153 | 5 | 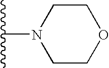 |

and pharmaceutically acceptable salts thereof.

Other illustrative examples of the compounds of Formula (I-153) include the compounds of Formula (Ib-153) as set forth below:

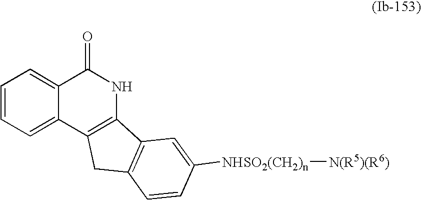

and pharmaceutically acceptable salts thereof,

| Compound | n | —N(R⁵)(R⁶) |
|---|---|---|
| 3a-153 | 1 | —N(CH₃)₂ |
| 3b-153 | 2 | —N(CH₃)₂ |
| 3c-153 | 3 | —N(CH₃)₂ |
| 3d-153 | 4 | —N(CH₃)₂ |
| 3e-153 | 5 | —N(CH₃)₂ |
| 4a-153 | 1 | 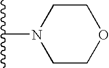 |
| 4b-153 | 2 | 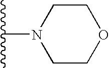 |
| 4c-153 | 3 | 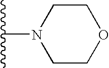 |
| 4d-153 | 4 | 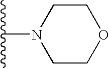 |
| 4e-153 | 5 | 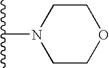 |

and pharmaceutically acceptable salts thereof.

Other illustrative examples of the compounds of Formula (I-153) include the compounds of Formula (Ic-153) as set forth below:

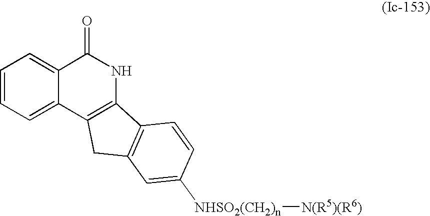

and pharmaceutically acceptable salts thereof,

| Compound | n | —N(R⁵)(R⁶) |
|---|---|---|
| 5a-153 | 1 | —N(CH₃)₂ |
| 5b-153 | 2 | —N(CH₃)₂ |
| 5c-153 | 3 | —N(CH₃)₂ |
| 5d-153 | 4 | —N(CH₃)₂ |
| 5e-153 | 5 | —N(CH₃)₂ |
| 6a-153 | 1 | 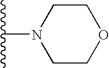 |
| 6b-153 | 2 | 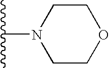 |
| 6c-153 | 3 | 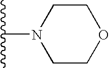 |
| 6d-153 | 4 | 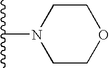 |
| 6e-153 | 5 | 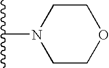 |

and pharmaceutically acceptable salts thereof.

Other illustrative examples of the compounds of Formula (I-153) include the compounds of Formula (Id-153) as set forth below:

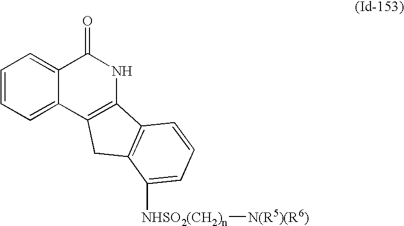

and pharmaceutically acceptable salts thereof,

| Compound | n | —N(R⁵)(R⁶) |
|---|---|---|
| 7a-153 | 1 | —N(CH₃)₂ |
| 7b-153 | 2 | —N(CH₃)₂ |
| 7c-153 | 3 | —N(CH₃)₂ |
| 7d-153 | 4 | —N(CH₃)₂ |
| 7e-153 | 5 | —N(CH₃)₂ |
| 8a-153 | 1 | 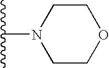 |
| 8b-153 | 2 | 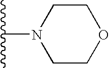 |
| 8c-153 | 3 | 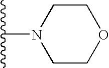 |
| 8d-153 | 4 | 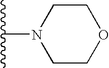 |
| 8e-153 | 5 | 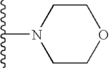 |

and pharmaceutically acceptable salts thereof.

### 4.8 Compounds of Formula (I-154)

The present invention provides methods for treating or preventing erectile dysfunction or urinary incontinence, comprising administering to a subject in need thereof an effective amount of a compound according to Formula (I-154), below: 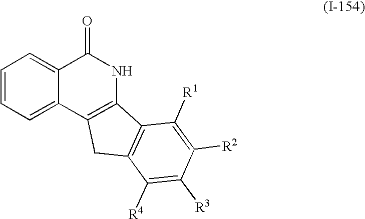
or a pharmaceutically acceptable salt thereof,
wherein:
R¹, R², R³ and R⁴ are as defined above for the compounds of Formula (I-154).

In one embodiment, R¹ is —NH(CH₂)ₙ—N(R⁵)(R⁶) and R², R³ and R⁴ are each hydrogen.

In still another embodiment, R⁴ is —NH(CH₂), —N(R⁵)(R⁶) and R₁, R² and R³ are each hydrogen.

In one embodiment, R₅ and R₆ are each C₁-C₆ alkyl.

In another embodiment, R⁵ and R⁶ are each methyl.

In one embodiment, n is 1.

In another embodiment, n is 2.

In still another embodiment, n is 3.

In yet another embodiment, n is 4.

In a further embodiment, n is 5.

In various embodiments, —N(R⁵)(R⁶) is:

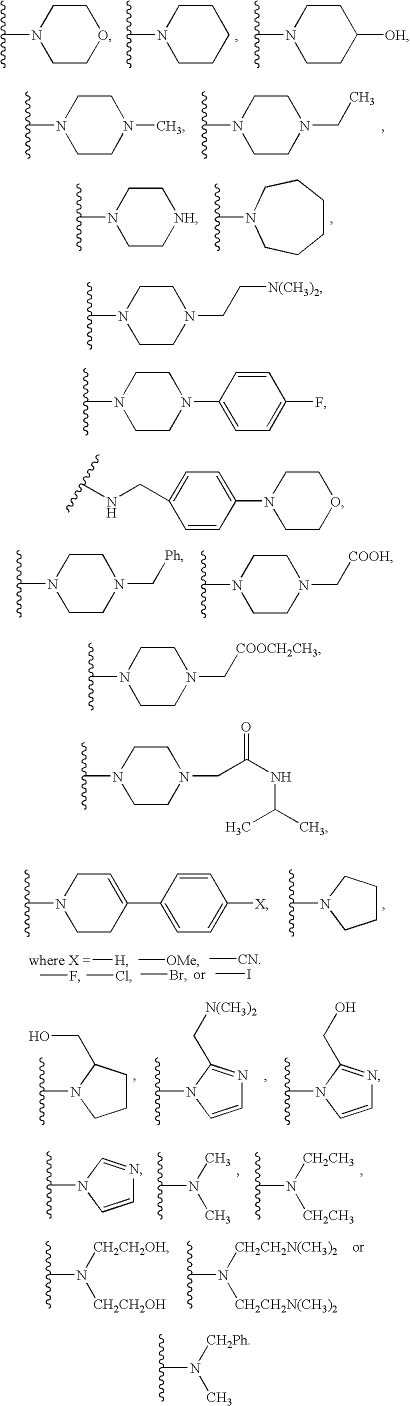

Illustrative examples of the compounds of Formula (I-154) include the compounds of Formula (Ia-154) as set forth below:

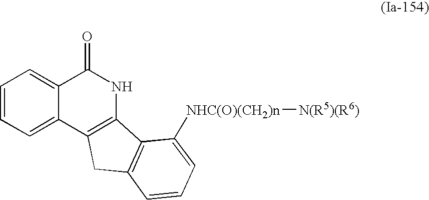

and pharmaceutically acceptable salts thereof,

| Compound | n | —N(R⁵)(R⁶) |
|---|---|---|
| 1a-154 | 1 | —N(CH₃)₂ |
| 1b-154 | 2 | —N(CH₃)₂ |
| 1c-154 | 3 | —N(CH₃)₂ |
| 1d-154 | 4 | —N(CH₃)₂ |
| 1e-154 | 5 | —N(CH₃)₂ |
| 2a-154 | 1 | 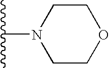 |
| 2b-154 | 2 | 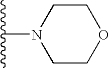 |
| 2c-154 | 3 | 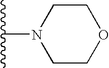 |
| 2d-154 | 4 | 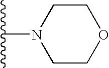 |
| 2e-154 | 5 | 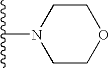 |

and pharmaceutically acceptable salts thereof.

Other illustrative examples of the compounds of Formula (I-154) include the compounds of Formula (Ib-154) as set forth below:

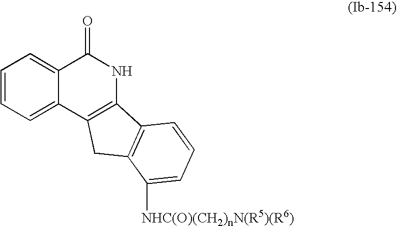

and pharmaceutically acceptable salts thereof,

| Compound | n | —N(R⁵)(R⁶) |
|---|---|---|
| 3a-154 | 1 | —N(CH₃)₂ |
| 3b-154 | 2 | —N(CH₃)₂ |
| 3c-154 | 3 | —N(CH₃)₂ |
| 3d-154 | 4 | —N(CH₃)₂ |
| 3e-154 | 5 | —N(CH₃)₂ |
| 4a-154 | 1 | 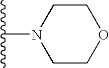 |
| 4b-154 | 2 | 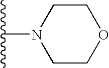 |
| 4c-154 | 3 | 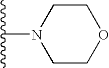 |
| 4d-154 | 4 | 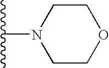 |
| 4e-154 | 5 | 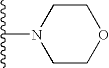 |

and pharmaceutically acceptable salts thereof.

### 4.9 Compound of Formula (II-154)

The present invention provides methods for treating or preventing erectile dysfunction or urinary incontinence, comprising administering to a subject in need thereof an effective amount of a compound according to Formula (II-154), below: 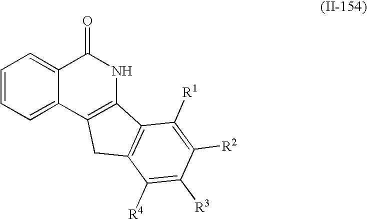
or a pharmaceutically acceptable salt thereof,
wherein:
R¹, R², R³ and R⁴ are as defined above for the compounds of Formula (II-154).

In one embodiment, R¹ is —NHC(O)—(CH₂), —N(Z₁)(Z₂) and R₂, R³ and R⁴ are each hydrogen.

In another embodiment, R² is —NHC(O)—(CH₂)ₙ—N(Z₁)(Z₂) and R¹, R³ and R⁴ are each hydrogen.

In another embodiment, R₃ is —NHC(O)—(CH₂)ₙ—N(Z₁)(Z₂) and R₁, R² and R⁴ are each hydrogen.

In still another embodiment, R⁴ is —NHC(O)—(CH₂), —N(Z₁)(Z₂) and R₁, R² and R³ are each hydrogen.

In one embodiment, n is 1.

In another embodiment, n is 2.

In still another embodiment, n is 3.

In yet another embodiment, n is 4.

In a further embodiment, n is 5.

In various embodiments, —N(Z₁)(Z₂) is:

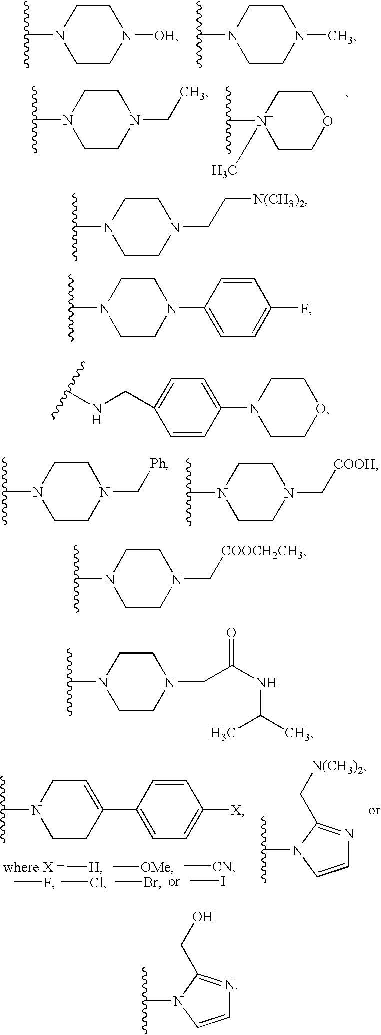

Illustrative examples of the compounds of Formula (II-154) include the compounds of Formula (IIa-154) as set forth below:

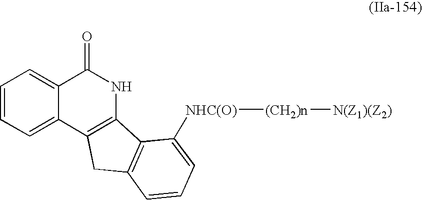

and pharmaceutically acceptable salts thereof,

| Compound | n | —N(Z₁)(Z₂) |
|---|---|---|
| 5a-154 | 1 | 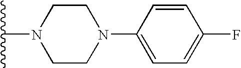 |
| 5b-154 | 2 | 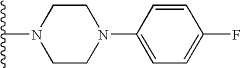 |
| 5c-154 | 3 | 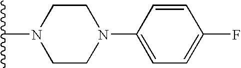 |
| 5d-154 | 4 | 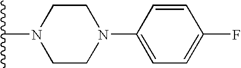 |
| 5e-154 | 5 | 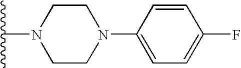 |
| 6a-154 | 1 | 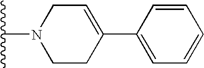 |
| 6b-154 | 2 | 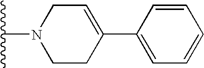 |
| 6c-154 | 3 | 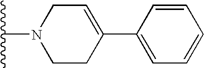 |
| 6d-154 | 4 | 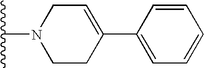 |
| 6e-154 | 5 | 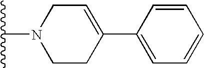 |
| 7a-154 | 1 | 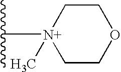 |
| 7b-154 | 2 | 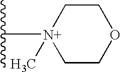 |
| 7c-154 | 3 | 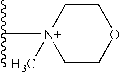 |
| 7d-154 | 4 | 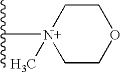 |
| 7e-154 | 5 | 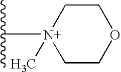 |

and pharmaceutically acceptable salts thereof.

Other illustrative examples of the compounds of Formula (II-154) include the compounds of Formula (IIb-154) as set forth below:

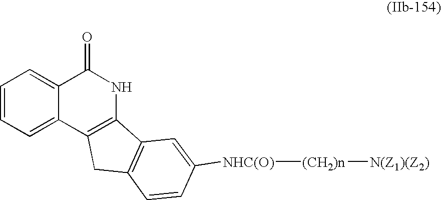

and pharmaceutically acceptable salts thereof,

| Compound | n | —N(Z₁)(Z₂) |
|---|---|---|
| 8a-154 | 1 | 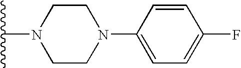 |
| 8b-154 | 2 | 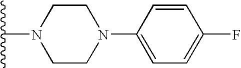 |
| 8c-154 | 3 | 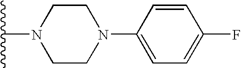 |
| 8d-154 | 4 | 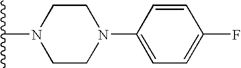 |
| 8e-154 | 5 | 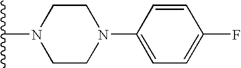 |
| 9a-154 | 1 | 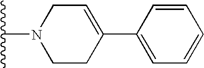 |
| 9b-154 | 2 | 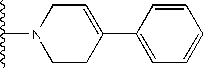 |
| 9c-154 | 3 | 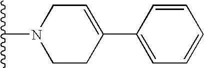 |
| 9d-154 | 4 | 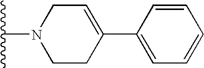 |
| 9e-154 | 5 | 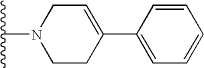 |
| 10a-154 | 1 | 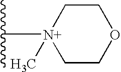 |
| 10b-154 | 2 | 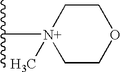 |
| 10c-154 | 3 | 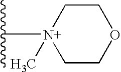 |
| 10d-154 | 4 | 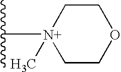 |
| 10e-154 | 5 | 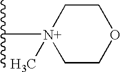 |

and pharmaceutically acceptable salts thereof.

Other illustrative examples of the compounds of Formula (II-154) include the compounds of Formula (IIc-154) as set forth below:

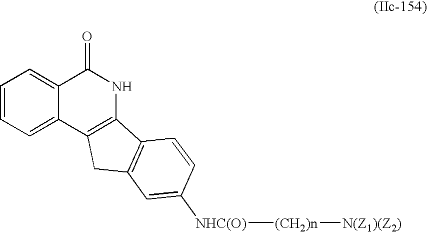

and pharmaceutically acceptable salts thereof,

| Compound | n | —N(Z₁)(Z₂) |
|---|---|---|
| 11a-154 | 1 | 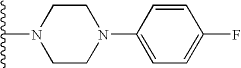 |
| 11b-154 | 2 | 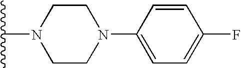 |
| 11c-154 | 3 | 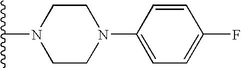 |
| 11d-154 | 4 | 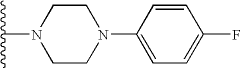 |
| 11e-154 | 5 | 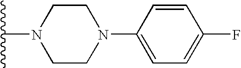 |
| 12a-154 | 1 | 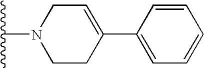 |
| 12b-154 | 2 | 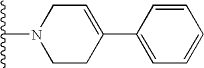 |
| 12c-154 | 3 | 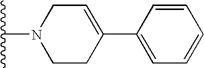 |
| 12d-154 | 4 | 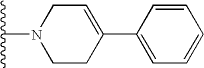 |
| 12e-154 | 5 | 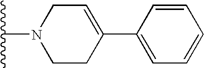 |
| 13a-154 | 1 | 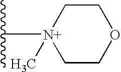 |
| 13b-154 | 2 | 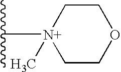 |
| 13c-154 | 3 | 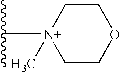 |
| 13d-154 | 4 | 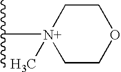 |
| 13e-154 | 5 | 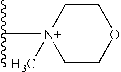 |

and pharmaceutically acceptable salts thereof.

Other illustrative examples of the compounds of Formula (II-154) include the compounds of Formula (IId-154) as set forth below:

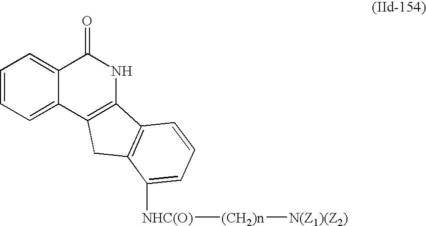

and pharmaceutically acceptable salts thereof,

| Compound | n | —N(Z₁)(Z₂) |
|---|---|---|
| 14a | 1 | 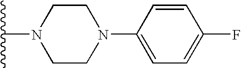 |
| 14b-154 | 2 | 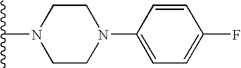 |
| 14c-154 | 3 | 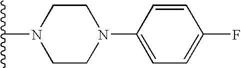 |
| 14d-154 | 4 | 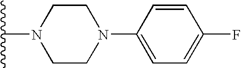 |
| 14e-154 | 5 | 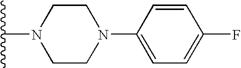 |
| 15a-154 | 1 | 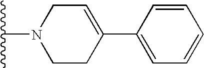 |
| 15b-154 | 2 | 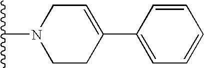 |
| 15c-154 | 3 | 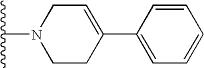 |
| 15d-154 | 4 | 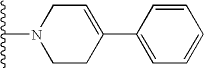 |
| 15e-154 | 5 | 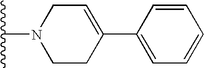 |
| 16a-154 | 1 | 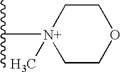 |
| 16b-154 | 2 | 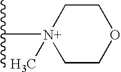 |
| 16c-154 | 3 | 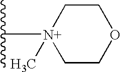 |
| 16d-154 | 4 | 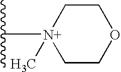 |
| 16e-154 | 5 | 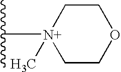 |

and pharmaceutically acceptable salts thereof.

### 4.10 Compounds of Formula (II-123)

As stated above, the present invention encompasses methods for treating or preventing erectile dysfunction or urinary incontinence, comprising administering to a subject in need thereof an effective amount of a compound of Formula (II-123): 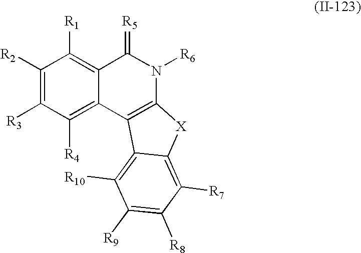
or a pharmaceutically acceptable salt thereof,
where R₁, R₂, R₃, R₄, R₅, R₆, R₇, R₈, R₉, R₁₀, and X are defined above for the compounds of Formula (II-123).

In one embodiment, R₁-R₄, R₇ and R₁₀ are hydrogen.

In another embodiment, at least one of R₁, R₂, R₃, R₄, R₇, R₈, R₉ and R₁₀ is other than hydrogen.

In another embodiment R₇-R₁₀ are hydrogen.

In still another embodiment, R₆ is hydrogen.

In one embodiment R₁-R₄ and R₇-R₁₀ is other than —O—(C₁-C₅ alkyl), and -A-B is other than —O—(C₁-C₁₀ alkyl).

In another embodiment compounds of Formula (II-123) have the structure of 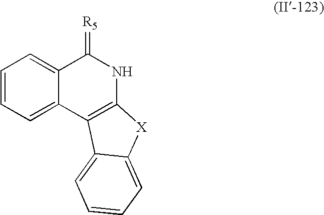
and pharmaceutically acceptable salts thereof,
wherein X and R₅ are defined above for Formula (II-123).

In other illustrative embodiments R₅ and X of Formula (II′-123) are as set forth below:

| R₅ | X |
|---|---|
| NH | —C(O)— |
| NH | —S— |
| NH | —NH— |
| NH | —CH₂— |
| NH | —N(SO₂Y)— |
| S | —C(O)— |
| S | —S— |
| S | —NH— |
| S | —CH₂— |
| S | —N(SO₂Y)— |
| O | —C(O)— |
| O | —S— |
| O | —NH— |
| O | —CH₂— |
| O | —N(SO₂Y)— |

pharmaceutically acceptable salts thereof.

As stated above, the present invention encompasses methods for treating or preventing erectile dysfunction or urinary incontinence, comprising administering to a subject in need thereof an effective amount of a compound of Formula (IIa-123): 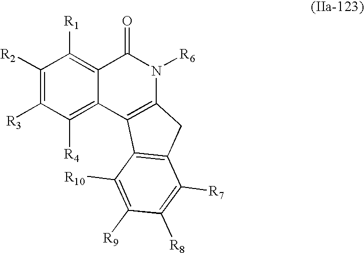
or a pharmaceutically acceptable salt thereof,
where R₁, R₂, R₃, R₄, R₆, R₇, R₈, R₉, and R₁₀ are defined above for the compounds of Formula (IIa-123).

In one embodiment, R₁-R₄ are hydrogen.

In one embodiment, R₁-R₄, R₇, R₉, and R₁₀ are hydrogen.

In another embodiment, at least one of R₁, R₂, R₃, R₄, R₇, R, R₉ and R₁₀ is other than hydrogen.

In still another embodiment, Rᵣ is hydrogen.

In one embodiment R₁-R₄ and R₇-R₁₀ is other than —O—(C₁-C₅ alkyl), and -A-B is other than —O—(C₁-C₁₀ alkyl).

In another embodiment compounds of Formula (IIa-123) have the structure of Formula (IIa′-123): 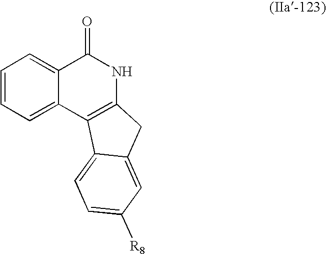
and pharmaceutically acceptable salts thereof,
wherein R₈ is defined above for Formula (IIa-123).

In one embodiment R₈ is -A-B, where -A- is —SO₂— and —B is —NZ₁Z₂ or —(C₁-C₅alkylene)-NZ₁Z₂.

Illustrative compounds of Formula (IIa′-123) are set forth below:

| Compound No. | R₈ |
|---|---|
| 9a-123 | —H |
| 11a-123 | —SO₂NH₂(CH₂)₃—(N-morpholinyl) |

and pharmaceutically acceptable salts thereof.

### 4.11 Compounds of Formula (Vi-123)

As stated above, the present invention encompasses methods for treating or preventing erectile dysfunction or urinary incontinence, comprising administering to a subject in need thereof an effective amount of a compound of Formula (VI-123): 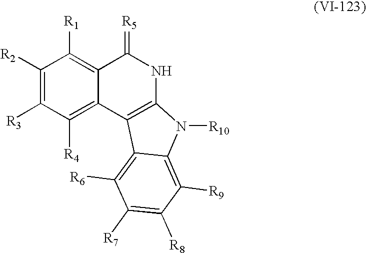
or a pharmaceutically acceptable salt thereof,
where R₁, R₂, R₃, R₄, R₅, R₆, R₇, R₈, R₉, and R₁₀ are defined above for the compounds of Formula (VI-123).

In one embodiment, R₁-R₄ are hydrogen. In another embodiment R₆, R₇, and R₉ are hydrogen. In another embodiment R₆-R₉ are hydrogen.

In another embodiment, at least one of R₁, R₂, R₃, R₄, R₆, R₇, R₈, and R₉ is other than hydrogen.

In one embodiment R₁-R₄ and R₆-R₉ is other than —O—(C₁-C₅ alkyl), and -A-B is other than —O—(C₁-C₁₀ alkyl).

In another embodiment compounds of Formula (VI-123) have the structure of Formula (VI′): 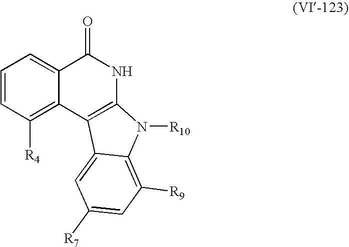
and pharmaceutically acceptable salts thereof,
wherein R₄, R₇, R₉, and R₁₀ are defined above for Formula (VI-123).

Illustrative compounds of Formula (VI′-123) are set forth below:

| Compound | R₄ | R₇ | R₉ | R₁₀ |
|---|---|---|---|---|
| VI′-1-123 | —H | —H | —H | —H |
| VI′-2-123 | —H | —H | —H | —CH₃ |
| V1′-3-123 | —H | —H | —H | —CH₂CH₃ |
| VI′-4-123 | —H | —H | —H | —CH₂COO CH₂CH₃ |
| VI′-5-123 | —H | —H | —H | —CH₂COOH |
| VI′-6-123 | —H | —H | —H | —CH₂CONHCH₃ |
| VI′-7-123 | —H | —H | —H | —CH₂Ph |
| VI′-8-123 | —H | —H | —H | —COOCH₃ |
| VI′-9-123 | —H | —H | —H | —SO₂NH₂ |
| VI′-10-123 | —H | —H | —H | —COOtBu |
| VI′-11-123 | —H | —H | —H | —COO CH₂CH₃ |
| VI′-12-123 | —H | —H | —H | —COCH₃ |
| VI′-13-123 | —H | —H | —H | —CONHCH₃ |
| VI′-14-123 | —H | —H | —H | —CONH CH₂CH₃ |
| VI′-15-123 | —H | —H | —H | —CONH(CH₂)₂N(CH₃)₂ |
| VI′-16-123 | —H | —H | —H | —CONH(CH₂)₂—(N-morpholinyl) |
| VI′-17-123 | —H | —H | —H | —CONH(CH₂)₃—(N-morpholinyl) |
| VI′-18-123 | —H | —H | —H | —CONH(CH₂)₂COO CH₂CH₃ |
| VI′-19-123 | —H | —H | —H | —CONH(CH₂)₂COOH |
| VI′-20-123 | —H | —H | —H | —CONH(CH₂)₂CONHCH₃ |
| VI′-21-123 | —H | —H | —H | —CONH-piperidine-1-yl |
| VI′-22-123 | —H | —H | —H | —CONH—(N-morpholinyl) |
| VI′-23-123 | —H | —H | —H | —CO(CH₂)₂-tetrazole-5-yl |
| VI′-24-123 | —H | —H | —NHCOCH₂N(CH₃)₂ | —H |
| VI′-25-123 | —H | —H | —SO₂NH(CH₂)₃—(N-morpholinyl) | —H |
| VI′-26-123 | —H | —NHCOCH₂N(CH₃)₂ | —H | —COOCH₃ |
| VI′-27-123 | —H | —SO₂NH(CH₂)₃—(N- | —H | —COOCH₃ |
| | | morpholinyl) | | |
| VI′-28-123 | —H | —H | —NHCOCH₂NMe₂ | —CONHCH₃ |
| VI′-29-123 | —H | —H | —SO₂NH(CH₂)₃—(N-morpholinyl) | —CONHCH₃ |
| VI′-30-123 | —NH₂ | —H | —NHCOCH₂N(CH₃)₂ | —CONHCH₃ |
| VI′-31-123 | —OH | —SO₂NH(CH₂)₃—(N- | —H | —CONHCH₃ |
| | | morpholinyl) | | |
| VI′-32-123 | —F | —NHCOCH₂N(CH₃)₂ | —H | —CONHCH₃ |
| VI′-33-123 | —OMe | —H | —SO₂NH(CH₂)₃—(N-morpholinyl) | —CONHCH₃ |

and pharmaceutically acceptable salts thereof.

### 4.10 Definitions

The following definitions are used in connection with the compounds of the invention:

“C₁-C₅ alkyl” refers to a straight or branched chain saturated hydrocarbon containing 1-5 carbon atoms. Examples of a C₁-C₅ alkyl group include, but are not limited to, methyl, ethyl, propyl, butyl, pentyl, isopropyl, isobutyl, sec-butyl and tert-butyl, isopentyl and neopentyl.

“C₁-C₆ alkyl” refers to a straight or branched chain saturated hydrocarbon containing 1-6 carbon atoms. Examples of a C₁-C₆ alkyl group include, but are not limited to, methyl, ethyl, propyl, butyl, pentyl, hexyl, isopropyl, isobutyl, sec-butyl and tert-butyl, isopentyl and neopentyl, n-hexyl, sec-hexyl, tert-hexyl, iso-hexyl, neohexyl.

“C₁-C₈ alkyl” refers to a straight or branched chain saturated hydrocarbon containing 1-8 carbon atoms. Examples of a C₁-C₈ alkyl group include, but are not limited to, methyl, ethyl, propyl, butyl, pentyl, hexyl, heptyl, octyl, isopropyl, isobutyl, sec-butyl and tert-butyl, isopentyl, neopentyl, isohexyl, isoheptyl and isooctyl.

“C₁-C₁₀ alkyl” refers to a straight or branched chain saturated hydrocarbon containing 1-10 carbon atoms. Examples of a C₁-C₁₀ alkyl group include, but are not limited to, methyl, ethyl, propyl, butyl, pentyl, hexyl, heptyl, octyl, -nonyl, decyl, isopropyl, isobutyl, sec-butyl and tert-butyl, isopentyl, neopentyl, isohexyl, isoheptyl, isooctyl, isononyl and isodecyl.

“C₂-C₁₀ alkenyl” refers to a straight or branched chain unsaturated hydrocarbon containing 2-10 carbon atoms and at least one double bond. Examples of a C₂-C₁₀ alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, isohexene, 1-heptene, 2-heptene, 3-heptene, 1-octene, 2-octene, 3-octene, 4-octene, 1-nonene, 2-nonene, 3-nonene, 4-nonene, 1-decene, 2-decene, 3-decene, 4-decene and 5-decene.

“C₂-C₁₀ alkynyl” refers to a straight or branched chain unsaturated hydrocarbon containing 2-10 carbon atoms and at least one triple bond. Examples of a C₂-C₁₀ alkynyl group include, but are not limited to, acetylene, propyne, 1-butyne, 2-butyne, isobutyne, sec-butyne, 1-pentyne, 2-pentyne, isopentyne, 1-hexyne, 2-hexyne, 3-hexyne, isohexyne, 1-heptyne, 2-heptyne, 3-heptyne, 1-octyne, 2-octyne, 3-octyne, 4-octyne, 1-nonyne, 2-nonyne, 3-nonyne, 4-nonyne, 1-decyne, 2-decyne, 3-decyne, 4-decyne and 5-decyne.

“C₁-C₅ alkylene” refers to a C₁-C₅ alkyl group in which one of the C₁-C₅ alkyl group's hydrogen atoms has been replaced with a bond. Examples of a C₁-C₅ alkylene include —CH₂—, —CH₂CH₂—, —CH₂CH₂CH₂—, —CH₂CH₂CH₂CH₂—, and —CH₂CH₂CH₂CH₂CH₂—.

“Halo-substituted C₁-C₅ alkyl” refers to a C₁-C₅ alkyl group, as defined above, wherein one or more of the C₁-C₅ alkyl group's hydrogen atoms has been replaced with —F, —Cₗ, —Br or —I. Representative examples of an alkylhalo group include, but are not limited to, —CH₂F, —CCl₃, —CF₃, —CH₂Cₗ, —CH₂CH₂Br, —CH₂CH₂I, —CH₂CH₂CH₂F, —CH₂CH₂CH₂Cl, —CH₂CH₂CH₂CH₂Br, —CH₂CH₂CH₂CH₂I, —CH₂CH₂CH₂CH₂CH₂Br, —CH₂CH₂CH₂CH₂CH₂I, —CH₂CH(Br)CH₃, —CH₂CH(Cₗ)CH₂CH₃, —CH(F)CH₂CH₃ and —C(CH₃)₂(CH₂Cl).

“Amino-substituted C₁-C₅ alkyl” refers to a C₁-C₅ alkyl group, as defined above, wherein one or more of the C₁-C₅ alkyl group's hydrogen atoms has been replaced with —NH₂. Representative examples of an amino-substituted C₁-C₅ alkyl group include, but are not limited to, —CH₂NH₂, —CH₂CH₂NH₂, —CH₂CH₂CH₂NH₂, —CH₂CH₂CH₂CH₂NH₂, —CH₂CH₂CH₂CH₂CH₂NH₂, —CH₂CH(NH₂)CH₃, —CH₂CH(NH₂)CH₂CH₃, —CH(NH₂)CH₂CH₃ and —C(CH₃)₂(CH₂NH₂).

“Aryl” refers to a phenyl or pyridyl group. Examples of an aryl group include, but are not limited to, phenyl, N-pyridyl, 2-pyridyl, 3-pyridyl and 4-pyridyl. An aryl group can be unsubstituted or substituted with one or more of the following groups: —C₁-C₅ alkyl, halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H or C₁-C₁₀ alkyl. Unless indicated otherwise, an aryl group is unsubstituted.

“H₂NC(O)-substituted aryl” refers to an aryl group, as defined above, wherein one of the aryl group's hydrogen atoms has been replaced with one or more —C(O)NH₂ groups. Representative examples of a dH₂NC(O)-substituted aryl group) include 2-C(O)NH₂-phenyl, 3-C(O)NH₂-phenyl, 4-C(O)NH₂-phenyl, 2-C(O)NH₂-pyridyl, 3-C(O)NH₂-pyridyl and 4-C(O)NH₂-pyridyl.

“—(C₁-C₅ alkyl)-(3- to 7-membered monocyclic heterocycle)” refers to a C₁-C₅ alkyl group, as defined above, wherein one of the C₁-C₅ alkyl group's hydrogen atoms has been replaced with a -3- to 7-membered monocyclic heterocycle. Representative examples of a —(C₁-C₅ alkyl)-(3- to 7-membered monocyclic heterocycle) group include, but are not limited to, —CH₂CH₂-morpholine, —CH₂CH₂-piperidine, —CH₂CH₂CH₂-morpholine and —CH₂CH₂CH₂-imidazole.

“Hydroxy-substituted C₁-C₅ alkyl” refers to a C₁-C₅ alkyl group, as defined above, wherein one of the C₁-C₅ alkyl group's hydrogen atoms has been replaced with a hydroxyl group. Representative examples of -(hydroxy-substituted C₁-C₅ alkyl groups) include, but are not limited to, —CH₂OH, —CH₂CH₂OH, —CH₂CH₂CH₂OH, —CH₂CH₂CH₂CH₂OH, —CH₂CH₂CH₂CH₂CH₂OH, —CH₂CH(OH)CH₃, —CH₂CH(OH)CH₂CH₃, —CH(OH)CH₂CH₃ and —C(CH₃)₂CH₂OH.

“Carboxy-substituted-(C₁-C₅ alkyl)” refers to a C₁-C₅ alkyl group, as defined above, wherein one of the C₁-C₅ alkyl group's hydrogen atoms has been replaced with a —COOH group. Representative examples of an alkylcarboxy group include, but are not limited to, —CH₂COOH, —CH₂CH₂COOH, —CH₂CH₂CH₂COOH, —CH₂CH₂CH₂CH₂COOH, —CH₂CH(COOH)CH₃, —CH₂CH₂CH₂CH₂CH₂COOH, —CH₂CH(COOH)CH₂CH₃, —CH(COOH)CH₂CH₃ and —C(CH₃)₂CH₂COOH.

A “C₃-C₈ monocyclic cycloalkyl” is a non-aromatic, saturated hydrocarbon ring containing 3-8 carbon atoms. Representative examples of a C₃-C₈ monocyclic cycloalkyl include, but are not limited to, cyclopropyl, cyclobutyl, cyclopentyl, cyclohexyl, cycloheptyl and cyclooctyl. A C₃-C₈ monocyclic cycloalkyl can be unsubstituted or independently substituted with one or more of the following groups: —C₁-C₅ alkyl, halo, -(halo-substituted C₁-C₅ alkyl), hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H or C₁-C₁₀ alkyl. Unless indicated otherwise, a C₃-C₈ monocyclic cycloalkyl group is unsubstituted.

A “3- to 7-membered monocyclic heterocycle” refers to a monocyclic 3- to 7-membered aromatic or non-aromatic monocyclic cycloalkyl in which 1-4 of the ring carbon atoms have been independently replaced with a N, O or S atom. The 3- to 7-membered monocyclic heterocycles can be attached via a nitrogen, sulfur, or carbon atom. Representative examples of a 3- to 7-membered monocyclic heterocycle group include, but are not limited to, piperidinyl, piperazinyl, morpholinyl, pyrrolyl, oxazinyl, thiazinyl, diazinyl, triazinyl, tetrazinyl, imidazolyl, tetrazolyl, pyrrolidinyl, isoxazolyl, furanyl, furazanyl, pyridinyl, oxazolyl, thiazolyl, thiophenyl, pyrazolyl, triazolyl, and pyrimidinyl.

A “7- to 10-membered bicyclic heterocycle” refers to a bicyclic 7- to 10-membered aromatic or non-aromatic bicyclic cycloalkyl in which 1-4 of the ring carbon atoms have been independently replaced with a N, O or S atom. The 7- to 10-membered bicyclic heterocycles can be attached via a nitrogen, sulfur, or carbon atom. Representative examples of a 7- to 10-membered bicyclic heterocycle group include, but are not limited to, benzimidazolyl, indolyl, isoquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl.

A “nitrogen-containing 3- to 7-membered monocyclic heterocycle” refers to a 3- to 7-membered monocyclic heterocycle, defined above, which contains at least one ring nitrogen atom. The nitrogen-containing 3- to 7-membered monocyclic heterocycles can be attached via a nitrogen, sulfur, or carbon atom. Representative examples of nitrogen-containing-3- to 7-membered monocyclic heterocycles include, but are not limited to, piperidinyl, piperazinyl, pyrrolyl, oxazinyl, thiazinyl, diazinyl, triazinyl, tetrazinyl, imidazolyl, tetrazolyl, pyrrolidinyl, isoxazolyl, pyridinyl, oxazolyl, thiazolyl, pyrazolyl, triazolyl, pyrimidinyl, and morpholinyl.

A “nitrogen-containing 7- to 10-membered bicyclic heterocycle” refers to a 7- to 10-membered bicyclic heterocycle, defined above, which contains at least one ring nitrogen atom. The nitrogen-containing 7- to 10-membered bicyclic heterocycles can be attached via a nitrogen, sulfur, or carbon atom. Representative nitrogen-containing 7- to 10-membered bicyclic heterocycles include -quinolinyl, -isoquinolinyl, -chromonyl, -indolyl, -isoindolyl, -indolizinyl, -indazolyl, -purinyl, -4H-quinolizinyl, -isoquinolyl, -quinolyl, -phthalazinyl, -naphthyridinyl -carbazolyl, -β-carbolinyl and the like.

“Halo” is —F, —Cl, —Br or —I.

In connection with the term “-A-B,” the compound's “A” group should be construed from left to right. For example, when “A” is “—SO₂NH—,” then the “B” group forms a bond with the “A” group nitrogen, and not sulfur, atom.

A “subject” is a mammal, for example, a human, mouse, rat, guinea pig, dog, cat, horse, cow, pig, or non-human primate, such as a monkey, chimpanzee, baboon or rhesus. In one embodiment, a subject is a human.

The phrase “pharmaceutically acceptable salt,” as used herein, is a salt of an acid and a basic nitrogen atom of a compound of the invention. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, and pamoate (i.e., 1,1′-methylene-bis-(2-hydroxy-3-naphthoate)) salts. The term “pharmaceutically acceptable salt” also refers to a salt of a compound of the invention having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like. The term “pharmaceutically acceptable salt” also includes a hydrate of a compound of the invention.

An “effective amount” when used in connection a compound of the invention is an amount that is effective for treating or preventing erectile dysfunction or urinary incontinence.

The following abbreviations are used herein and have the indicated definitions: DMF is N,N-dimethylformamide, DMSO is dimethylsulfoxide, EtOAc is ethyl acetate, EtOH is ethanol, HPLC is high pressure liquid chromatography, Me is methyl, MeCN is acetonitrile, MeOH is methanol, MS is mass spectrometry, Ms is mesyl (methanesulfonyl), NEt₃ is triethylamine, NMR is nuclear magnetic resonance, PARP is poly(ADP-ribose)polymerase, Tf is triflyl (trifluoromethanesulfonyl), TFA is trifluoroacetic acid, THF is tetrahydrofuran; TLC is thin layer chromatography, and Ts is tosyl (p-toluenesulfonyl).

The compounds of Formulas (I-149), (IV-149), (I-152), (II-152), (I-153), (I-154), (II-154), (II-123), (IIa-123), and (VI-123) can exist in a keto or enol tautomeric form. This invention encompasses both the keto and enol forms of these compounds. Although the present application depicts the keto form of the compounds of Formulas (I-149), (IV-149), (I-152), (II-152), (I-153), (I-154), (II-154), (II-123), (IIa-123), and (VI-123), the referenced Formulas encompass both the keto and enol forms.

### 4.12 Methods for Using the Compounds of the Invention

The compounds of the invention are useful for treating or preventing erectile dysfunction. Erectile dysfunction includes an inability to achieve or maintain a full erection, particularly that which is sufficient to achieve or maintain sexual intercourse. The inability can be a total inability, an inconsistent ability, or a tendency to maintain only a brief erection. Erectile dysfunction that is treatable or preventable according to the methods described herein includes idiopathic erectile dysfunction, as well as that which can result, for example, from trauma, including mechanical trauma, particularly that resulting from surgery, to the nerves (such as during prostatectomy); diabetes mellitus; a cardiovascular disease, including atherosclerosis; radiation; or certain drugs. The erectile dysfunction can also be age-related.

In one embodiment the erectile dysfunction results from prostate surgery.

In a further embodiment the erectile dysfunction results from prostate nerve injury.

The compounds of the invention are also useful for treating or preventing urinary incontinence. Urinary incontinence that is treatable or preventable according to the methods described herein, can result, for example, from trauma, including mechanical trauma, particularly during childbirth or that resulting from surgery, to the nerves (such as during prostatectomy or gynecological surgery); diabetes mellitus; a cardiovascular disease, including atherosclerosis; radiation; or certain drugs. The urinary incontinence can also be age-related.

In one embodiment the subject in need of urinary incontinence treatment or prevention is male.

In one embodiment the subject in need of urinary incontinence treatment or prevention is female.

### Therapeutic/Prophylactic Administration

Administration of a compound of the invention can be accomplished via any mode of administration for prophylactic or therapeutic agents. These modes include systemic or local administration such as oral, nasal, parenteral, transdermal, subcutaneous, buccal, rectal, or topical administration.

In one embodiment, a compound of the invention can be administered as a component of a composition that also comprises a physiologically acceptable carrier or vehicle.

Depending on the intended mode of administration, the compositions can be in solid, semi-solid or liquid dosage form, such as, for example, injectables, tablets, suppositories, pills, time-release capsules, elixirs, tinctures, emulsions, syrups, powders, liquids, suspensions, or the like, which can be in unit dosages and consistent with conventional pharmaceutical practices. Likewise, they can also be administered in intravenous (both bolus and infusion), intraperitoneal, subcutaneous or intramuscular form, all using forms well known to those skilled in the pharmaceutical arts.

Illustrative pharmaceutical compositions include tablets and gelatin capsules comprising a compound of the invention and a physiologically acceptable carrier or vehicle, such as a) a diluent, for example, lactose, dextrose, sucrose, mannitol, sorbitol, cellulose, sodium, saccharin, glucose and/or glycine; b) a lubricant, for example, silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, for example, magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, for example, starches, agar, methyl cellulose, bentonite, xanthan gum, algiic acid or its sodium salt, or effervescent mixtures; and/or e) absorbent, colorant, flavorant and sweetener.

Liquid, particularly injectable, compositions can, for example, be prepared by dissolution or dispersion. For example, the compound of the invention is dissolved in or mixed with a physiologically acceptable solvent such as, for example, water, saline, aqueous dextrose, glycerol, ethanol, and the like, to thereby form an injectable isotonic solution or suspension.

The compounds of the invention can be also formulated as a suppository that can be prepared from fatty emulsions or suspensions using polyalkylene glycols such as propylene glycol, as the carrier.

The compounds of the invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564.

Compounds of the invention can also be delivered by the use of monoclonal antibodies as individual carriers to which the compounds of the invention are coupled. The compounds of the invention can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the compounds of the invention can be coupled to a class of biodegradable polymers useful for achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Parental injectable administration is generally used for subcutaneous, intramuscular, or intravenous injections and infusions. Injectables can be prepared in conventional forms, either as liquid solutions or suspensions or solid forms suitable for dissolving in liquid prior to injection.

One embodiment, for parenteral administration employs the implantation of a slow-release or sustained-released system, according to U.S. Pat. No. 3,710,795, incorporated herein by reference.

In one embodiment, an effective amount of a compound of the invention is formulated within an implant. In another embodiment the implant can be a bladder, penile or prostate implant.

The compositions can be sterilized or contain non-toxic amounts of adjuvants, such as preserving, stabilizing, wetting or emulsifying agents, solution promoters, salts for regulating the osmotic pressure pH buffering agents, and other substances, including, but not limited to, sodium acetate or triethanolamine oleate. In addition, they can also contain other therapeutically useful substances.

Compositions can be prepared according to conventional mixing, granulating or coating methods, respectively, and the present pharmaceutical compositions can contain from about 0.1% to about 99%, preferably from about 1% to about 70% of the compound of the invention by weight or volume.

The dosage regimen utilizing the compound of the invention can be selected in accordance with a variety of factors including type, species, age, weight, and medical condition of the subject; the severity of the erectile dysfunction or urinary incontinence to be treated; the route of administration; the renal or hepatic function of the subject; and the particular compound of the invention employed. A person skilled in the art can determine the effective amount of the compound of the invention effective for treating or preventing erectile dysfunction or urinary incontinence.

The amount of the compound of the invention that is effective for treating or preventing erectile dysfunction or urinary incontinence can be determined by standard clinical techniques. In addition, in vitro or in vivo assays can optionally be employed to help identify optimal dosage ranges. The precise dose to be employed can also depend on the route of administration, and the seriousness of the condition being treated and can be decided according to the judgment of a health-care practitioner. Suitable effective dosage amounts, however, range from about 10 micrograms to about 5 grams about every 4 h, although they can be about 500 mg or less per every 4 hours. In one embodiment the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, and about 5.0 g, every 4 hours. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one compound of the invention is administered, the effective dosage amounts correspond to the total amount administered.

The amount of a compound of the invention that is effective for treating or preventing erectile dysfunction or urinary incontinence will typically range from about 0.01 mg/kg to about 100 mg/kg of body weight per day, in one embodiment, from about 0.1 mg/kg to about 50 mg/kg body weight per day, and in another embodiment, from about 1 mg/kg to about 20 mg/kg of body weight per day.

Furthermore, compounds of the invention can be administered in intranasal form via topical use of suitable intranasal vehicles, or via transdermal routes, using those forms of transdermal skin patches well known to those skilled in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of the compound of the invention ranges from about 0.1% to about 15%, w/w or w/v.

In one embodiment, the compounds of the invention are administered to a subject prior to, during, or subsequent to undergoing surgery, particularly prostate surgery.

### 4.13 Methods for Making the Compounds of Formulas (I-149), (II-149), (III-149) and (IV-149)

Examples of synthetic pathways useful for making the compounds of Formulas (I-149), (II-149), (III-149), and (IV-149) are set forth in Example 1-149 below and generalized in Schemes (1-149)-(10-149).

Methods useful for making compounds of Formula (I-149) wherein X is —CH₂— and R₅ is O, and of Formula (IV-149) wherein X is —CH₂—, are illustrated below in Scheme 1-149.

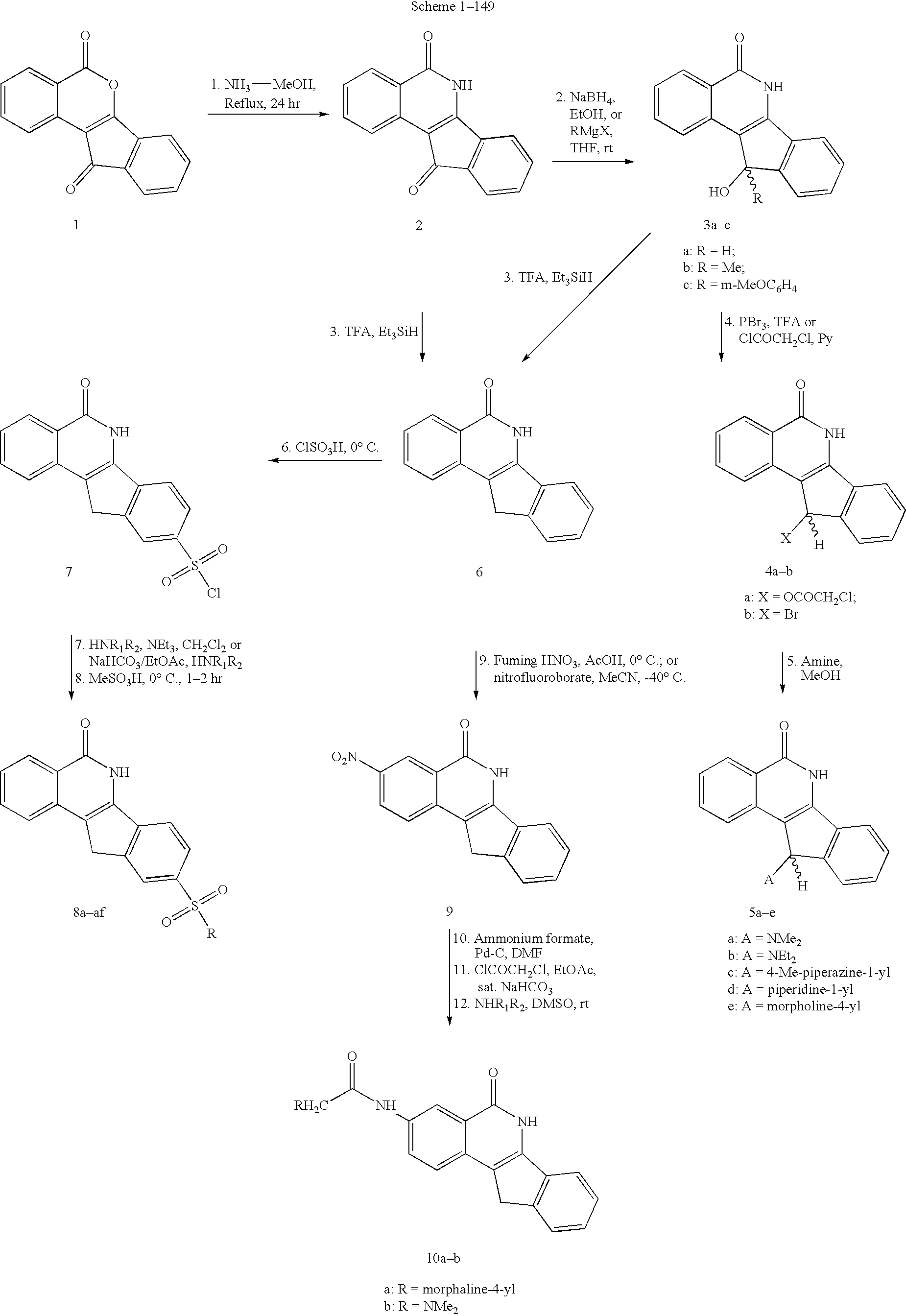

wherein compounds 8a-149-8af-149 are as follows:

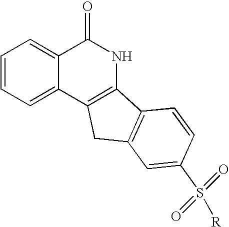

8a-149-8af-149

a. R = 4-Methyl-piperazin-1-yl
b. R = 4-CH₂CO₂Me-piperazin-1-yl
c. R = 4-CH₂CO₂OH-piperazin-1-yl
d. R = imidazol-1-yl
e. R = L-prolinol
f. R = morpholin-4-yl
g. R = —NHCH₂CH₂NMe₂
h. R = —NHCH₂CH₂-pipendin-1-yl
i. R = —NHCH₂CH₂N-(pyridin-2-yl)
j. R = —NHCH₂CH₂-morpholin-4-yl
k. R = —NHCH₂CH₂-(2-N-Me-tetrahydropyrrolidin-1-yl)
l. R = —NHCH₂CH₂CH₂-morpholin-4-yl
m. R = —NHCH₂CH₂CH₂-(tetrahydropyrrolidin-1-yl)
n. R = —NHCH₂CH₂CH₂-imidazol-1-yl
a. R = —NHCH₂CH₂CH₂-(4-methylpiperazin-1-yl)
p. R = —N(CH₂CH₂NEt₂)₂
q. R = —N(CH₂CH₂NMe₂)₂
r. R = —N(CH₂CH₂OH)₂
s. R = —NHCH₂CH₂CN
t. R = —NHC(NH)NH₂
u. R = —NH[4-(1,2,4-triazole)]
v. R = —NH[4-(morpholin-4-yl)phenyl]
w. R = —NHCH₂CH₂(4-N-benzylpiperidine)
x. R = —NHCH₂CH₂(2-thienyl)
y. R = —NH[1-(4-azabenzimidazole)]
z. R = —NH[1-(4-(2′-pyridyl)piperazine)]
aa. R = —NHCH₂CH₂N[CH₂CH₂OH]₂
ab. R = —NH[1-(4-benzylpiperazine)]
ac. R = —NH₂
ad. R = —NHCH₂CH₂Ph
ae. R = —NHCH₂CH₂[4-OMe(phenyl)]
af. R = —NHC(O)(morpholin-4-yl)

5,6-dihydro-5,11-diketo-11H-isoquinoline (2) was prepared by reacting compound 1 (Aldrich Chemical, Milwaukee, Wis.) with ammonia in methanol.

(±) 11-hydroxy-5,6-dihydro-5-oxo-11H-indeno[1,2-c]isoquinoline (3a) was prepared by reacting 2 with NaBH₄ in ethanol.

(±) 11-hydroxy-11-methyl-5,6-dihydro-5-oxo-11H-isoquinoline (3b) was prepared by reacting 2 with MeMgI.

(±) 11-hydroxy-11-(m-methoxyphenyl)-5,6-dihydro-5-oxo-1H-indeno[1,2-c]isoquinoline (3c) was prepared from 2 using m-MeO—C₆H₄MgI.

(±) 11-N,N-dimethylamino-5,6-dihydro-5-oxo-1H-indeno[1,2-c]isoquinoline (5a) was prepared from 3a using chloroacetylchloride followed by reacting with dimethylamine. Similarly prepared are: (±) 11-N,N-diethylamino-5,6-dihydro-5-oxo-11H-indeno[1,2-c]isoquinoline (5b), (±) 11-N-(piperidino-1-yl)-5,6-dihydro-5-oxo-1H-indeno[1,2c]isoquinoline (5d), (±) 11-N-(4-methylpiperazino-1-yl)-5,6-dihydro-5-oxo1 IH-indeno[1,2-c]isoquinoline (5c), (±) 11-N-(morpholino-4-yl)-5,6-dihydro-5-oxo11H-isoquinoline (5e). (±) 11-N-(morpholino-4-yl)-5,6-dihydro-5-oxo-11H-indeno[1,2-c]isoquinoline (5e) was also prepared from (±) 11-bromo-5,6-dihydro-5-oxo-11H-indeno[1,2-c]isoquinoline (4b).

5,6-Dihydro-5-oxo-11H-indeno-[1,2-c]isoquinoline (6) is prepared by reduction of 5,6-dihydro-5,11-diketo-11H-isoquinoline (2) or (±) 1-hydroxy-5,6-dihydro5-oxo-11H-isoquinoline (3a) using CF3COOH/triethylsilane. 9-Chlorosulphonyl-5,6-dihydro-5-oxo-11H-indeno-[1,2-c]isoquinoline (7) was prepared by chlorosulfonation of 5,6-dihydro-5-oxo-11H-indeno-[1,2-c]isoquinoline (6). 9-[N-(4-methylpiperazine-1 yl)sulphonyl]-5,6-dihydro-5-oxo-11H-indeno-[1,2-c]isoquinoline (8a-149) was prepared from 9-chlorosulphonyl-5,6-dihydro-5-oxo-1H-indeno-[1,2-c]isoquinoline (7), and N-methylpiperazine. Similarly prepared are: 9-[N-(4-carbomethoxymethylenepiperazino-1yl)sulphonyl]-5,6-dihydro-5-oxo-1H-indeno-[1,2-c]isoquinoline (8b-149), 9-[N-4-(2-hydroxyethylpiperazino-1-yl)-sulphonyl]-5,6-dihydro-5-oxo-1H-indeno-[1,2-c]isoquinoline (8c-149), 9-[N-(imidazolo-1-yl)sulphonyl]-5,6-dihydro-5-oxo-11H-isoquinoline (8d-149), 9-[N-(2-hydroxyprolinyl)sulphonyl]-5,6-dihydro-5-oxo-11H-indeno[1,2-c]isoquinoline (8e-149), 9-[N-morpholinesulphonyl]-5,6-dihydro-5-oxo-11H-indeno[1,2-c]isoquinoline (8f-149), 9-[N-(2-[N,N-dimethylamino]ethyl)aminosulphonyl]-5,6-dihydro-5-oxo-11H-indeno[1,2-c]isoquinoline (8g-149), 9-[N-(2-[piperidino-1-yl]ethyl)-aminosulphonyl]-5,6-dihydro-5-oxo-11H-indeno[1,2-c]isoquinoline (8h-149), 9-[N (2-(pyridino-2-yl)-ethyl)-aminosulphonyl]-5,6-dihydro-5-oxo-11H-indeno[1,2c]isoquinoline (8i-149), 9-[N-(2-[morpholino-4-yl]ethyl)-aminosulphonyl]-5,6-dihydro-5-oxo-11H-indeno[1,2-c]isoquinoline (8j-149), 9-[N-(2-[N-methyltetrahydropyrroiidino-1-yl]ethyl) aminosulphonyl]-5,6-dihydro-5-oxo-1H-indeno-[1,2-c]isoquinoline (8k-149), 9-[N-(3-[morpholino-4-yl] propyl)-aminosulphonyl]-5,6-dihydro-5-oxo-11H-indeno-[1,2c]isoquinoline (8l-149), 9-[N-(3-[tetrahydropyrrolodino-1-yl]propyl)aminosulphonyl]-5,6-dihydro-5-oxo-11H-indeno-[1,2-c]isoquinoline (8m⁻¹⁴⁹), 9-[N-(3-[imidazolo-1-yl]propyl)aminosulphonyl]-5,6-dihydro-5-oxo-11H-indeno-[1,2-c]isoquinoline (8n-149), 9-[N-[3-(4-methylpiperazino-1-yl]propyl)-aminosulphonyl]-5,6-dihydro-5-oxo-11H-indeno-[1,2c]isoquinoline (8o-149), 9-[N,N-di-(2-[N,N-diethylamino]ethyl)-aminosulphonyl]-5,6-dihydro-5-oxo-1H-indeno-[1,2-c]isoquinoline (8p-149), 9-[N,N di-(2-[N,N dimethylamino]ethyl)aminosulphonyl]-5,6-dihydro-5-oxo-11H-indeno-[1,2-c]isoquinoline (8q-149), and 9-[N,N-di(2-[N,N-dihydroxyethylamino] ethyl)-aminosulphonyl]-5,6-dihydro-5-oxo-11H-indeno-[1,2c]isoquinoline (8r-149).

Compounds 8s-149-8af-149 can be prepared using the methods described above for making compounds of 8a-149-8r-149, using appropriate amine intermediates.

Scheme 2-149 illustrates a method useful for making terminal carboxylic acid compounds of Formulas 8ag-149-8ao-149. This method comprises reacting sulfonyl chloride 7-149 with the alkyl ester of an amino acid in the presence of a base, preferably triethyamine, to provide an intermediate terminal carboxylic acid alkyl ester, which is then hydrolyzed using a base such as sodium hydroxide to provide the corresponding terminal carboxylic acid. 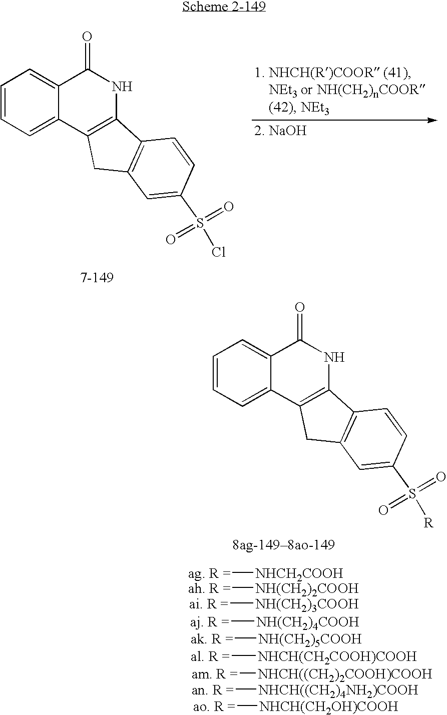
wherein:
R′ is -amino-substituted C₁-C₅ alkyl or -hydroxy-substituted C₁-C₅ alkyl
R″ is —C₁-C₆ alkyl; and
n is an integer ranging from 1 to 6.

### General Procedure for Making 9-sulfonamide carboxylic acid Derivatives of Scheme 2-149

### Preparation of 9-sulfonamido carboxylic acid ester

To a 0.5 M solution of an ester of Formula 41 or 42 in CH₂Cl₂ is added compound 7-149 (1.0 eq) and the resulting mixture is stirred for 5 minutes. Triethylamine (about 5 eq) is then added and the resulting reaction is stirred at room temperature and monitored using TLC or HPLC until complete. The reaction mixture is filtered, the solid is washed using MeOH to provide the intermediate 9-sulfonamido carboxylic acid ester which can be used without further purification.

### Ester Hydrolysis

To an approximately 0.5 M solution of a 9-sulfonamide carboxylic acid ester in ethanol is added about 3.0 N aqueous sodium hydroxide (about 5.0 eq) and the resulting reaction is refluxed if necessary and monitored using TLC or HPLC until completion. The reaction mixture is neutralized to about pH 7.0 using about 1.0 N HCl and the neutralized reaction mixture is extracted twice using EtOAc. The combined EtOAc layers are washed sequentially with water and saturated aqueous sodium chloride, then dried over sodium sulfate and concentrated in vacuo to afford a crude residue which is purified using flash column chromatography to provide the desired 9-sulfonamide carboxylic acid compound.

Acid hydrolysis with neat TFA can be useful where the sulfonamide has a t-butyl ester group.

In another embodiment, illustrated below in Scheme 3-149, compounds of general Formula 13-149 can be made by a method comprising contacting a compound of Formula 11 and a compound of Formula 12 in the presence of a base for a time and at a temperature sufficient to make a compound of Formula 13-149. 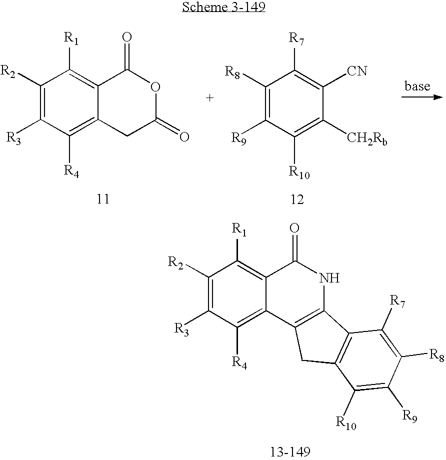
wherein:
R₁-R₄ and R₇-R₁₀ are as defined above for Formula (I-149); and
Rb is —Cl, —Br, —I, —OMs, —OTs or —OTf.

In one embodiment, Rb is —Br.

In another embodiment, Rb and Rd are both —Br.

In one embodiment, about 0.1 to about 10 equivalents of a compound of Formula 12 are used per about 1 equivalent of a compound of Formula 11.

In another embodiment, about 0.5 to about 5 equivalents of a compound of Formula 12 are used per about 1 equivalent of a compound of Formula 11.

In still another embodiment, about 1 to about 2 equivalents of a compound of Formula 12 are used per about 1 equivalent of a compound of Formula 11.

In one embodiment, about 1 to about 10 equivalents of base are used per about 1 equivalent of a compound of Formula 11.

In another embodiment, about 3 to about 7 equivalents of base are used per about 1 equivalent of a compound of Formula 11.

In a yet another embodiment, about 5 to about 6 equivalents of base are used per about 1 equivalent of a compound of Formula 11.

Suitable bases for use in the method of Scheme 3 are organic bases such as triethylamine, diisopropylamine, diisopropylethylamine, pyridine, lutidine and imidazole; and inorganic bases such as alkali metal carbonates, including sodium carbonate, potassium carbonate and cesium carbonate.

In one embodiment, the base is triethylamine.

In another embodiment, the base is potassium carbonate.

The method of Scheme 3 can be carried out in the presence of a solvent, such as acetonitrile, methylene chloride, chloroform, THF, DMF, DMSO, ethyl acetate, acetone, benzene, diethyl ether, water or mixtures thereof.

In one embodiment, the solvent is acetonitrile.

In another embodiment, the solvent is DMF.

In still another embodiment, where the solvent is not water, the solvent is substantially anhydrous, i.e., comprises less than about 1% water.

In one embodiment, the method of Scheme 3-149 is carried out for a time of about 0.5 hours to about 48 hours.

In another embodiment, the method of Scheme 3-149 is carried out for a time of about 3 hours to about 36 hours.

In still another embodiment, the method of Scheme 3-149 is carried out for a time of about 8 hours to about 24 hours.

In yet another embodiment, the method of Scheme 3-149 is carried out for a time of about 15 hours to about 20 hours.

In a further embodiment, the method of Scheme 3-149 is carried out at a temperature of about 0° C. to about 200° C.

In another embodiment, the method of Scheme 3-149 is carried out at a temperature of about 25° C. to about 150° C.

In yet another embodiment, the method of Scheme 3-149 is carried out at a temperature of about 50° C. to about 100° C.

General Procedure for the Preparation of Compounds of Formula 13-149

To a solution of a homophthalic anhydride of Formula 11 (about 1 equivalent) in a suitable solvent, such as acetonitrile, is added a compound of Formula 12 (about 1 to about 2 eq) followed by a suitable base, such as triethylamine (about 1 to about 5 eq). The resulting reaction is reaction is allowed to stir for about 1 hour, at which time a colored precipitate appears. The reaction is then heated at reflux for about 20 hours, cooled to room temperature and filtered. The collected solid is washed using acetonitrile and dried under vacuum to provide a compound of Formula 13-149.

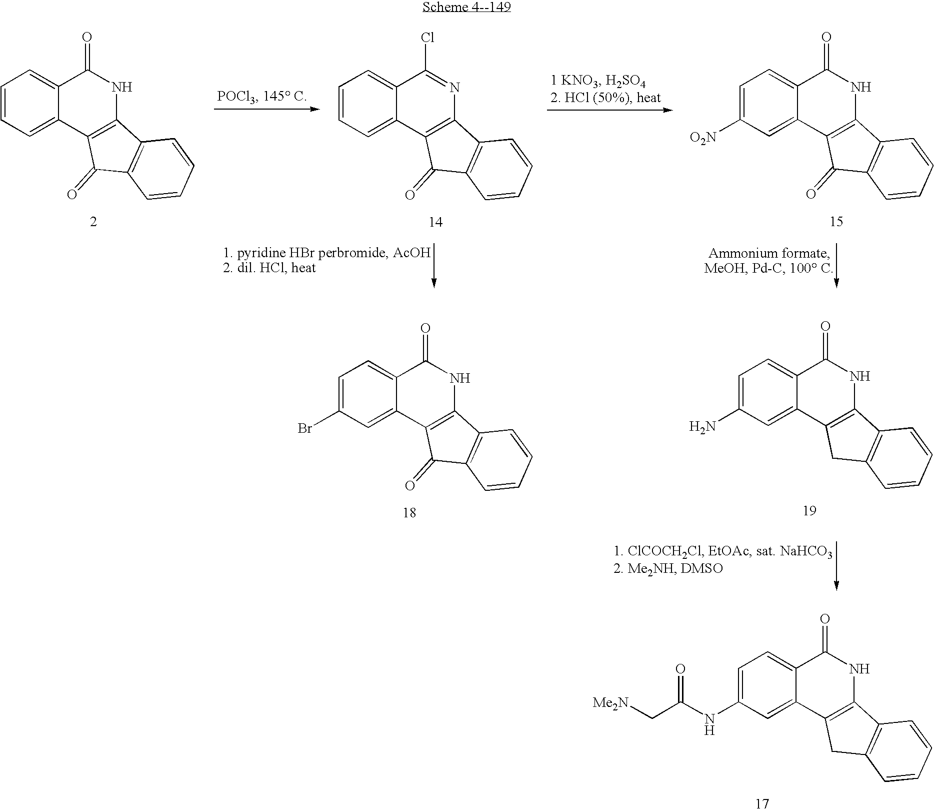

The amide derivative 2-dimethylamino-N-(5-oxo-5,11-dihydro-6H-indeno[1,2c]isoquinoiin-2-yl)-acetamide (17) was prepared from 5-chloro-11H-indeno 5 [1,2c]isoquinoline (14). Compound 14 was subjected to nitration to provide nitro compound 15, which was reduced using ammonium formate to provide amine 16, which was derivatized to acetamide 17, and followed by amination of the chloroacetamide intermediate. 2-bromo5,6-dihydro-5-oxo-11H-indeno[1,2-c]isoquinoline (18) was prepared by bromination of Compound 14.

Scheme 5-149 illustrates methods useful for making oxygen-substituted compounds of Formula (I-149), where R₅ and X are oxygen, and of Formula (IV-149), where X is oxygen. 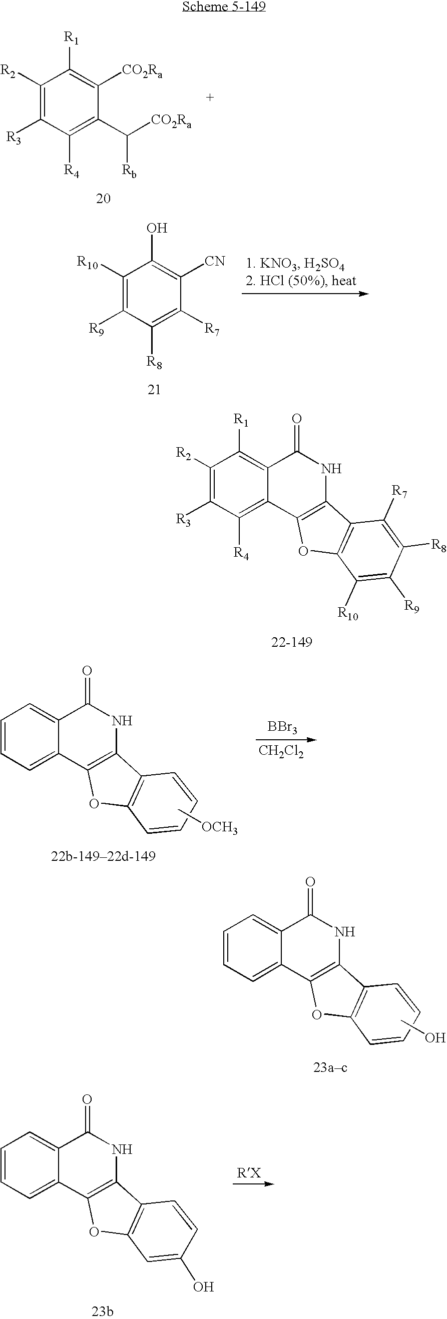 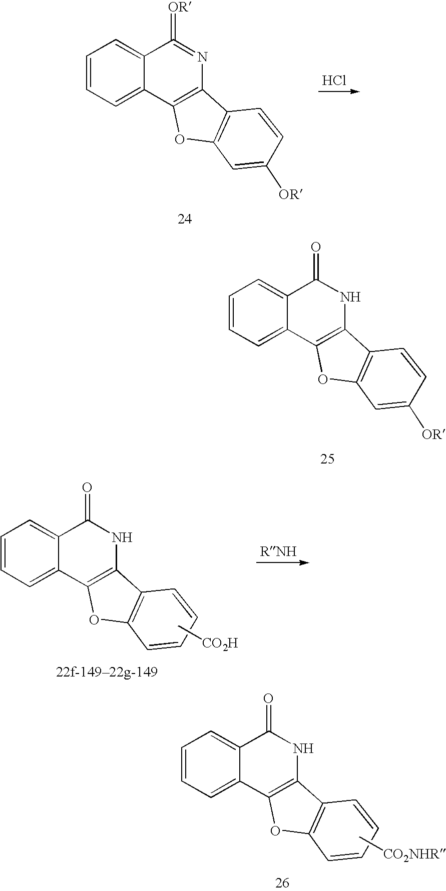
wherein:
R₁-R₅ are as defined above for Formula (I-149); each occurrence of Ra is independently C₁-C₅ alkyl;
Rb is —Cl, —Br, —I, —OMs, —OTs or —OTf;
R′ is —C₁-C₁₀ alkyl, alkanol or alkylcarboxy; and
R″ is —C₁-C₁₀ alkyl, aryl, heterocycle, alkanol or alkylcarboxy.

In one embodiment, Ra is methyl.

In another embodiment, Rb is —Br

In another embodiment, illustrated above in Scheme 5-149, compounds of Formula 22-149 can be made by a method comprising contacting a compound of Formula 20 and a compound of Formula 21 in the presence of a base for a time and at a temperature sufficient to make a compound of Formula 22-149.

In one embodiment, about 0.1 to about 10 equivalents of a compound of Formula 20 are used per about 1 equivalent of a compound of Formula 21.

In another embodiment, about 0.5 to about 5 equivalents of a compound of Formula 20 are used per about 1 equivalent of a compound of Formula 21.

In still another embodiment, about 1 to about 2 equivalents of a compound of Formula 20 are used per about 1 equivalent of a compound of Formula 21.

In one embodiment, about 1 to about 10 equivalents of base are used per about 1 equivalent of a compound of Formula 21.

In another embodiment, about 3 to about 7 equivalents of base are used per about 1 equivalent of a compound of Formula 21.

In a yet another embodiment, about 5 to about 6 equivalents of base are used per about 1 equivalent of a compound of Formula 21.

Suitable bases for use in the method are organic bases such as triethylamine, diisopropylamine, diisopropylethylamine, pyridine, lutidine and imidazole; and inorganic bases such as alkali metal carbonates such as sodium carbonate, potassium carbonate and cesium carbonate.

In one embodiment, the base is potassium carbonate.

In another embodiment, the base is triethylamine.

The method can be carried out in the presence of a solvent, such as acetonitrile, methylene chloride, chloroform, THF, DMF, DMSO, ethyl acetate, acetone, benzene, diethyl ether, water or mixtures thereof.

In one embodiment, the solvent is DMF.

In another embodiment, the solvent is acetonitrile.

In still another embodiment, the solvent is substantially anhydrous, i.e., comprises less than about 1% water.

In one embodiment, the method is carried out for a time of about 1 hour to about 96 hours.

In another embodiment, the method is carried out for a time of about 18 hours to about 72 hours.

In yet another embodiment, the method is carried out for a time of about 24 hours to about 48 hours.

In one embodiment, the method is carried out at a temperature of about 25° C. to about 200° C.

In another embodiment, the method is carried out at a temperature of about 50° C. to about 150° C.

In still another embodiment, the method is carried out at a temperature of about 75° C. to about 125° C.

Scheme 6-149 illustrates methods useful for making nitrogen-substituted compounds of the invention.

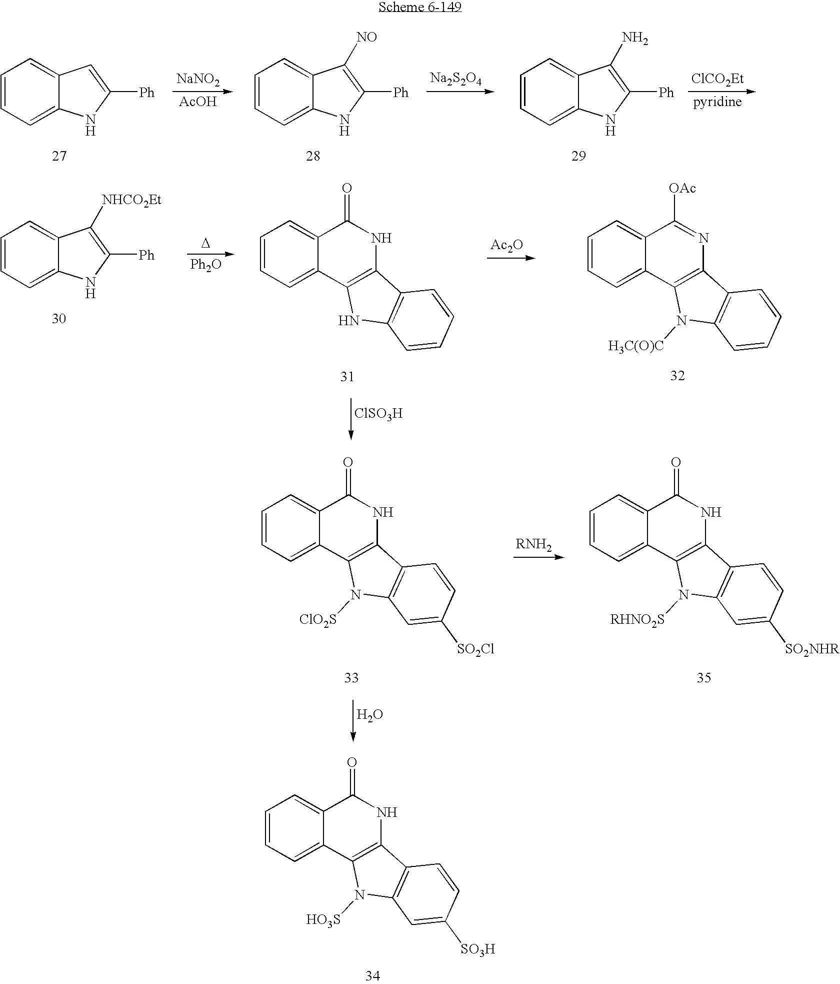

In an alternate embodiment, illustrated below in Scheme 7-149, nitrogen-substituted compounds of general Formula 37-149 can be made by a method comprising contacting a compound of Formula 36 and a compound of Formula 11 or Formula 20 in the presence of a base for a time and at a temperature sufficient to make a compound of Formula 37-149. 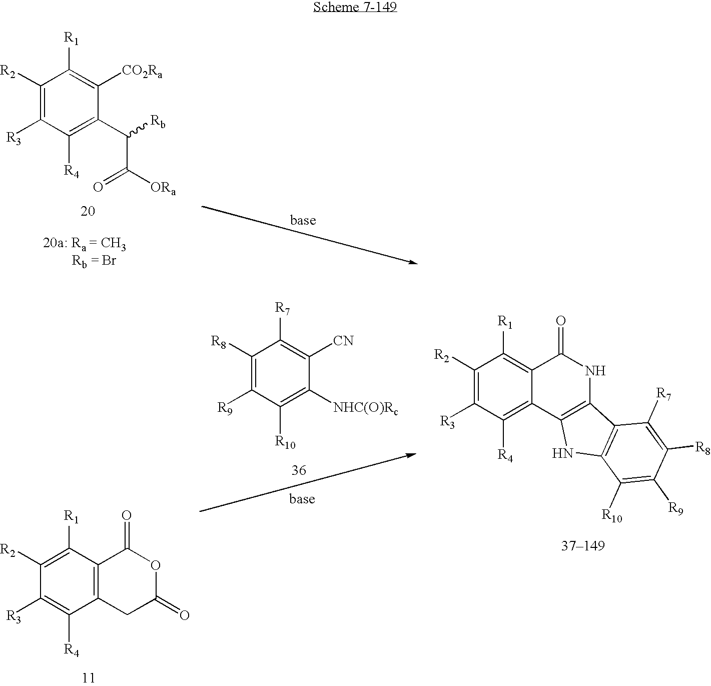
wherein:
R₁-R₄ and R₇-R₁₀ are as defined above for Formula (I-149);
each occurrence of Rₐ is independently C₁-C₅ alkyl;
R_{b} is —Cl, —Br, —I, —OMs, —OTs or —OTf, and
R_{c}, is C₁-C₅ alkyl.

In one embodiment, Rₐ is methyl.

In another embodiment, R_{b} is —Br.

In a further embodiment, Rₐ is methyl and R_{b} is —Br.

In still another embodiment, R_{c} is methyl.

In one embodiment, about 0.1 to about 10 equivalents of a compound of Formula 11 are used per about 1 equivalent of a compound of Formula 36.

In another embodiment, about 0.5 to about 5 equivalents of a compound of Formula 11 are used per about 1 equivalent of a compound of Formula 36.

In still another embodiment, about 1 to about 2 equivalents of a compound of Formula 11 are used per about 1 equivalent of a compound of Formula 36.

In one embodiment, about 0.1 to about 10 equivalents of a compound of Formula 20 are used per about 1 equivalent of a compound of Formula 36.

In another embodiment, about 0.5 to about 5 equivalents of a compound of Formula 20 are used per about 1 equivalent of a compound of Formula 36.

In still another embodiment, about 1 to about 2 equivalents of a compound of Formula 20 are used per about 1 equivalent of a compound of Formula 36.

In one embodiment, about 1 to about 10 equivalents of base are used per about 1 equivalent of a compound of Formula 36.

In another embodiment, about 3 to about 7 equivalents of base are used per about 1 equivalent of a compound of Formula 11.

In a yet another embodiment, about 5 to about 6 equivalents of base are used per about 1 equivalent of a compound of Formula 11.

Suitable bases for use in the method of Scheme 7-149 are organic bases such as triethylamine, diisopropylamine, diisopropylethylamine, pyridine, lutidine and imidazole; and inorganic bases such as alkali metal carbonates such as sodium carbonate, potassium carbonate and cesium carbonate.

In one embodiment, the base is potassium carbonate.

In another embodiment, the base is triethylamine.

The method of Scheme 7-149 can be carried out in the presence of a solvent, such as acetonitrile, methylene chloride, chloroform, THF, DMF, DMSO, ethyl acetate, acetone, benzene, diethyl ether, water or mixtures thereof.

In one embodiment, the solvent is DMF.

In another embodiment, the solvent is acetonitrile.

In still another embodiment, the solvent is substantially anhydrous, i.e., comprises less than about 1% water.

In one embodiment, the method of Scheme 7-149 is carried out for a time of about 1 hour to about 96 hours.

In another embodiment, the method of Scheme 7-149 is carried out for a time of about 18 hours to about 72 hours.

In yet another embodiment, the method of Scheme 7-149 is carried out for a time of about 24 hours to about 48 hours.

In one embodiment, the method of Scheme 7-149 is carried out at a temperature of about 25° C. to about 200° C.

In another embodiment, the method of Scheme 7-149 is carried out at a temperature of about 50° C. to about 150° C.

In still another embodiment, the method of Scheme 7-149 is carried out at a temperature of about 75° C. to about 125° C.

General Procedure for the Preparation of Compounds of Formula 37-149

### From a homophthalate

To a solution of a homophthalate of Formula 20 (about 1 eq) and an N-acylanthranilonitrile of Formula 36 (about 1 to about 2 eq) in a solvent such as DMF, under inert atmosphere, is added a base (about 5 eq), such as potassium carbonate and the reaction is allowed to stir for about 48 hours at about 100° C., then cooled to room temperature. The reaction mixture is then poured into about 1 N sodium hydroxide and the resulting solution is extracted with EtOAc. The EtOAc layer is washed sequentially with about 1 N HCl, saturated aqueous sodium chloride, dried over sodium sulfate, filtered and concentrated in vacuo. The resulting residue is dissolved using warming in toluene and the resulting solution is cooled to room temperature and precipitated using hexanes. The solid precipitate is filtered, washed using hexanes and dried in a vacuum oven at 50° C. for 72 h to provide a Compound of Formula 36.

The synthesis of phenyl amide 36, which is a useful intermediate in Scheme 7-149, is described below in Scheme 8-149. In this procedure, the amine group of a cyanoaniline compound of Formula 38 is acylated using an acyl chloride or an anhydride in the presence of an acid. 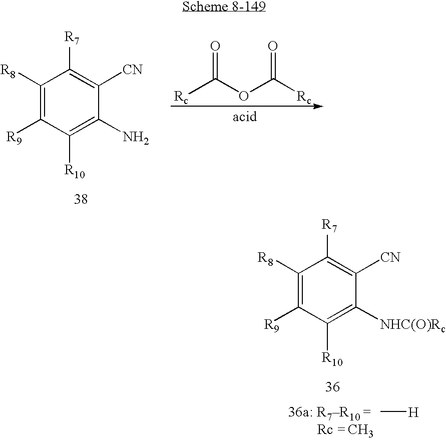
wherein:
R₇-R₁₀ are as defined above for Formula (I-149); and
R_{c} is C₁-C₅ alkyl.

Suitable acids for use in the method of Scheme 8-149 include, but are not limited to, sulfuric acid and phosphoric acid.

In one embodiment, the acid is sulfuric acid.

In another embodiment, R_{c} is methyl.

The method of Scheme 8-149 can be carried out in the presence of a solvent, including, but not limited to, acetonitrile, methylene chloride, chloroform, THF, DMF, DMSO, ethyl acetate, acetone, benzene, diethyl ether or mixtures thereof.

General Procedure for Making a Compound of Formula 36

To a solution of a compound of Formula 38 (about 1 eq) in acetic anhydride (about 6 eq) at 90° C. is added 1 drop of sulfuric acid (catalytic) and the resulting reaction is stirred at about 90° C. for about 2 h, and is then allowed to sit at room temperature for about 12 h. The reaction mixture is poured onto ice and the resulting solution is stirred for about 2 h, after which time the solution is neutralized to about pH 7.0 using 1 N sodium hydroxide. The resulting precipitate is filtered, washed using water (about 4×) and dried under vacuum for about 72 h to provide a compound of Formula 36.

In another embodiment, illustrated below in Scheme 9-149, sulfur substituted compounds of Formula 40-149 can be made by a method comprising contacting a compound of Formula 39 and a compound of Formula 11a or Formula 20 in the presence of a base for a time and at a temperature sufficient to make a compound of Formula 40-149. 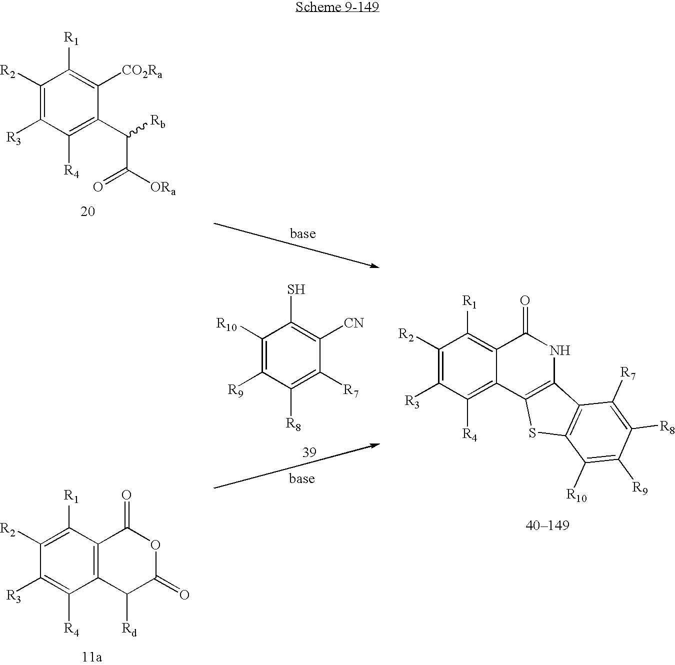
R₁-R₄ and R₇-R₁₀ are as defined above for Formula (I-149);
each occurrence of Rₐ is independently C₁-C₅ alkyl;
R_{b} is —Cl, —Br, —I, —OMs, —OTs or —OTf; and
R_{d} is —H or —Br.

In one embodiment, Rₐ is methyl.

In another embodiment, R_{b} is —Br.

In still another embodiment, Rₐ is methyl and R_{b} is —Br.

In yet another embodiment, R_{d} is —H.

In a further embodiment, R_{d} is —Br.

In one embodiment, about 0.1 to about 10 equivalents of a compound of Formula 11a are used per about 1 equivalent of a compound of Formula 39.

In another embodiment, about 0.5 to about 5 equivalents of a compound of Formula 11a are used per about 1 equivalent of a compound of Formula 39.

In still another embodiment, about 1 to about 2 equivalents of a compound of Formula 11a are used per about 1 equivalent of a compound of Formula 39.

In one embodiment, about 0.1 to about 10 equivalents of a compound of Formula 11a are used per about 1 equivalent of a compound of Formula 39.

In another embodiment, about 0.5 to about 5 equivalents of a compound of Formula 11a are used per about 1 equivalent of a compound of Formula 39.

In yet another embodiment, about 1 to about 2 equivalents of a compound of Formula 11a are used per about 1 equivalent of a compound of Formula 39.

In one embodiment, about 0.1 to about 10 equivalents of a compound of Formula 20 are used per about 1 equivalent of a compound of Formula 39.

In another embodiment, about 0.5 to about 5 equivalents of a compound of Formula 20 are used per about 1 equivalent of a compound of Formula 39.

In yet another embodiment, about 1 to about 2 equivalents of a compound of Formula 20 are used per about 1 equivalent of a compound of Formula 39.

In one embodiment, about 1 to about 10 equivalents of base are used per about 1 equivalent of a compound of Formula 39.

In another embodiment, about 3 to about 7 equivalents of base are used per about 1 equivalent of a compound of Formula 39.

In a yet another embodiment, about 5 to about 6 equivalents of base are used per about 1 equivalent of a compound of Formula 39.

Suitable bases for use in the method of Scheme 9-149 are organic bases such as triethylamine, diisopropylamine, diisopropylethylamine, pyridine, lutidine and imidazole; and inorganic bases such as alkali metal carbonates, including sodium carbonate, potassium carbonate and cesium carbonate.

In one embodiment, the base is potassium carbonate.

In another embodiment, the base is triethylamine.

The method of Scheme 9-149 can be carried out in the presence of a solvent, such as acetonitrile, methylene chloride, chloroform, THF, DMF, DMSO, ethyl acetate, acetone, benzene, diethyl ether, water or mixtures thereof.

In one embodiment, the solvent is DMF.

In another embodiment, the solvent is acetonitrile.

In one embodiment, the method of Scheme 9-149 is carried out for a time of about 1 hour to about 120 hours.

In another embodiment, the method of Scheme 9-149 is carried out for a time of about 24 hours to about 96 hours.

In yet another embodiment, the method of Scheme 9-149 is carried out for a time of about 60 hours to about 80 hours.

In one embodiment, the method of Scheme 9-149 is carried out at a temperature of about 0° C. to about 200° C.

In another embodiment, the method of Scheme 9-149 is carried out at a temperature of about 25° C. to about 150° C.

In still another embodiment, the method of Scheme 9-149 is carried out at a temperature of about 50° C. to about 100° C.

General Procedure for the Preparation Compounds of Formula 40-149

### From a homophthalic anhydride

A solution of a mercaptobenzonitrile of Formula 39 (about 1.0 eq) and a homophthalic anhydride of Formula 11a (about 2.0 eq) in a suitable solvent such as acetonitrile under inert atmosphere is warmed with stirring until all reactants are in solution. A suitable base such as triethylamine (about 1 to about 5 eq) is added and the reaction is allowed to stir at about 90° C. for about 72 hours, then cooled to room temperature. The reaction mixture is filtered, and the collected solid is washed using methanol, then dried in a vacuum oven at about 50° C. to provide a compound of Formula 40-149.

### From a homophthalate

A solution of a mercaptobenzonitrile of Formula 39 (about 1.0 eq) and a homophthalate of Formula 20 (about 2.0 eq) in a suitable solvent such as acetonitrile under inert atmosphere is warmed with stirring until all reactants are in solution. A suitable base such as triethylamine (about 1 to about 5 eq) is added and the reaction is allowed to stir at about 90° C. for about 72 hours, then cooled to room temperature. The reaction mixture is filtered, and the collected solid is washed using methanol, then dried in a vacuum oven at about 50° C. to provide a compound of Formula 40-149.

Methods for making compounds of Formula (IV-149) are illustrated below in Scheme 10-149. 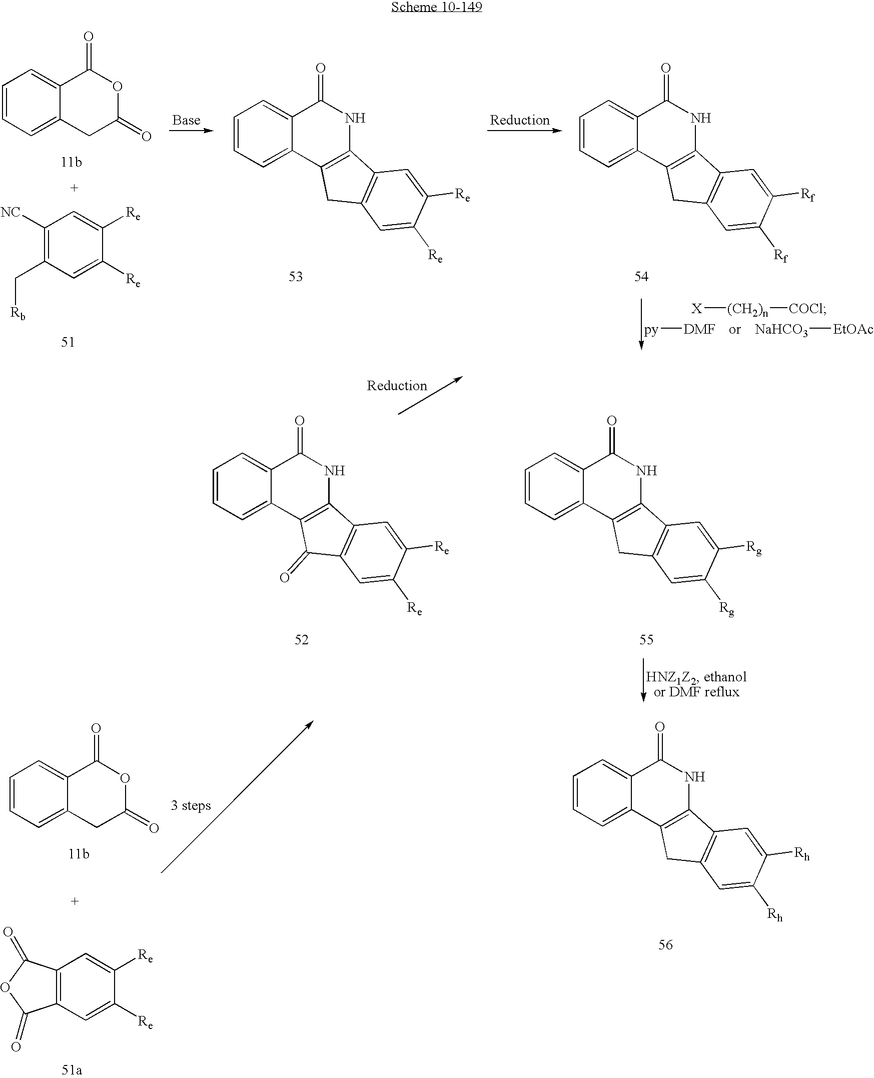
wherein: n, Z₁, and Z₂ as defined above for Formula (WV-146);
X is a leaving group such as bromide or chloride;
R_{b} is —Cl, —Br, —I, —OMs, —OTs, or —OTf;
one Rₑ is —H and the other Rₑ is —NO₂;
one R_{f} is —H and the other R_{f} is —NH₂;
one Rₑ is —H and the other Rₑ is —NHC(O)—(CH₂)ₙ—X; and
one Rₕ is —H and the other Rₕ is —NHC(O)—(CH₂)ₙ—NZ₁Z₂.

In one embodiment, R_{b} is —Br.

General Procedure for the Preparation of Compounds of Formula 56

To a solution of homophthalic anhydride (11b) (about 1 equivalent) in a suitable solvent, such as acetonitrile, is added a compound of Formula 51 (about 1 to about 2 equivalents), follwed by a suitable base, such as triethylamine (about 1 to about 5 equivalents). The resultant reaction mixture is allowed to stir for about 1 hour, at which time a precipitate appears. The reaction mixture is then heated to reflux for about 20 hours, cooled to room temperature and filtered. The collected solid is washed with acetonitrile and dried under vacuum to provide a compound of Formula 53.

Compound 52 can be prepared from homophthalic anhydride (11b) and benzoic anhydride in two steps. Homophthalic anhydride and benzoic anhydride are reacted in a suitable solvent such as pyridine in the presence of an acid such as HCl; subsequently reacted with acetic anhydride in pyridine and heated to reflux; and then refluxed in the presence of an amine such as NH₃ in MeOH; to provide the compound of Formula 52.

To a solution of the compound of Formula 52 or 53 in a suitable solvent, such as DMF, is added a reducing agent, such as ammonium formate in the presence of palladium on carbon. The reaction mixture is heated to a temperature of about 90 to 100° C., cooled to room temperature and filtered to provide a compound of the Formula 54.

The compound of the Formula 54 can be reacted with X—(CH₂)ₙ—COCl, under conditions effective to form an amide of the Formula 55.

The compound of Formula 55 can be reacted with an amine of Formula HNZ₁Z₂, in the presence of a solvent such as ethanol or DMF and heating to reflux, to form the compound of Formula 56.

### 4.14 Methods for Making the Compounds of Formulas (I-152) and (II-152)

Methods useful for making the compounds of Formulas (I-152) and (II-152) are generalized in Schemes 1-152 and 2-152. 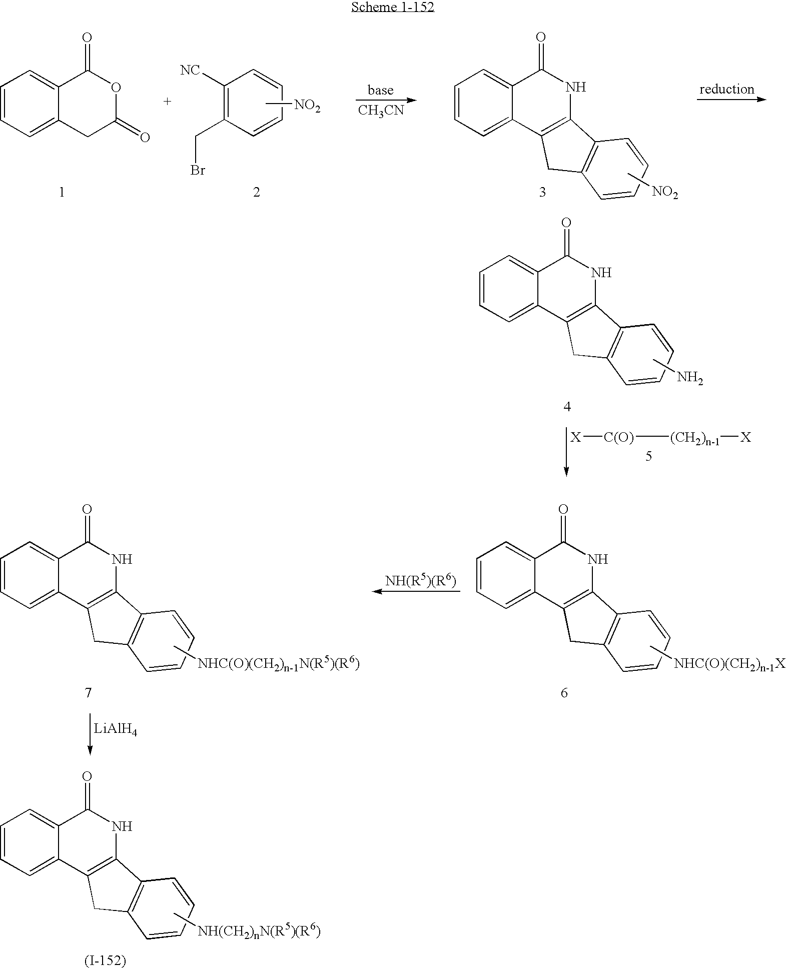
wherein each X is independently Cl or —Br, and n, R⁵ and R⁶ are defined above for the compounds of Formula (I-152).

Homophthalic anhydride 1 can be coupled with a nitrobenzene compound of Formula 2 in the presence of a base, for example, an amine base, to provide a tetracyclic nitro intermediate of Formula 3. The nitro group of 3 can be reduced using, for example, catalytic hydrogenation with a platinum or palladium catalyst, to provide an amino compound of Formula 4. A compound of Formula 4 can then be reacted with a stoichometric excess of an acid halide compound of Formula 5 to provide an amido compound of Formula 6. The chlorine or bromine atom of 6 can then be displaced by an amine of Formula NH(R⁵)(R⁶) to provide an amino compound of Formula 7. Finally, the amide moiety of a compound of Formula 7 can be reduced using lithium aluminum hydride to provide the compounds of Formula (I-152). 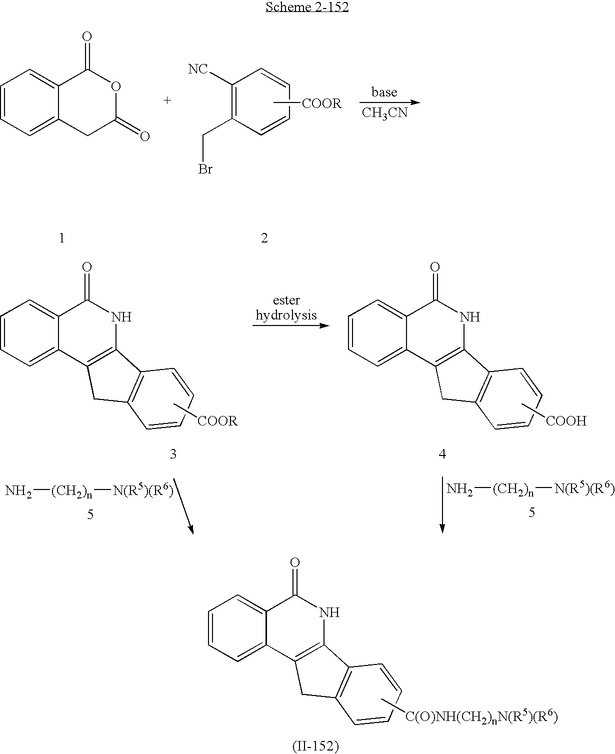
wherein R is methyl or ethyl, and n, R⁵ and R⁶ are defined above for the Compounds of Formula (II-152).

Homophthalic anhydride 1 can be coupled with a phenylester compound of Formula 2 in the presence of a base, for example, an amine base, to provide a tetracyclic nitro intermediate of Formula 3. The ester group of 3 can be hydrolyzed under basic or acidic conditions to provide an carboxylic acid compound of Formula 4. A compound of Formula 3 or formul 4 can then be coupled with a diaminoalkyl compound of Formula 5 (which is commercially available, or can be prepared by reacting dihaloalkyl compounds with various amines using methods well known to one of skill in the art of organic synthesis) to provide the compounds of Formula (II-152).

### 4.15 Methods for Making the Compounds of Formula (I-153)

Methods useful for making compounds of Formula (I-153) are set forth in the Example 3-153 below and generalized in Scheme 1-153. 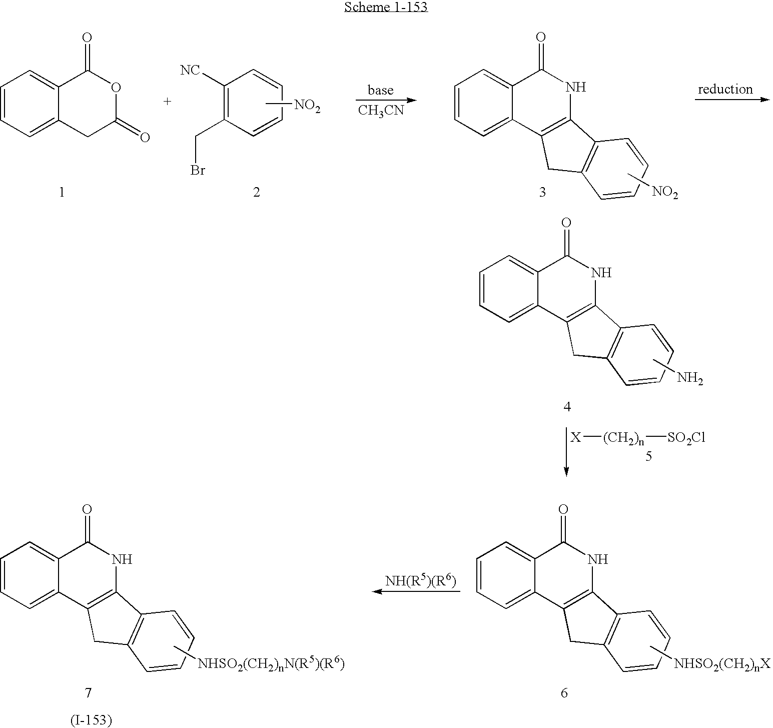
wherein X is —Cl or —Br, and n, R⁵ and R⁶ are defined above for the Compounds of Formula (I-153).

Homophthalic anhydride 1 can be coupled with a nitrobenzene compound of Formula 2 in the presence of a base, for example, an amine base, to provide a tetracyclic nitro intermediate of Formula 3. The nitro group of 3 can be reduced using, for example, catalytic hydrogenation with a platinum or palladium catalyst, to provide an amino compound of Formula 4. The amino group of 4 can be reacted with a sulfonyl chloride compound of Formula 5 to provide the chloro- or bromo-sulfonamide compounds of Formula 6. The chlorine or bromine atom of 6 can then be displaced by an amine of Formula NH(R₅)(R₆) to provide the compounds of Formula (I-153).

### 4.16 Methods for Making the Compounds of Formulas (I-154) and (II-154)

Methods useful for making the compounds of Formulas (I-154) and (II-154) are set forth in Example 4-154 below and generalized in Scheme (1-154). 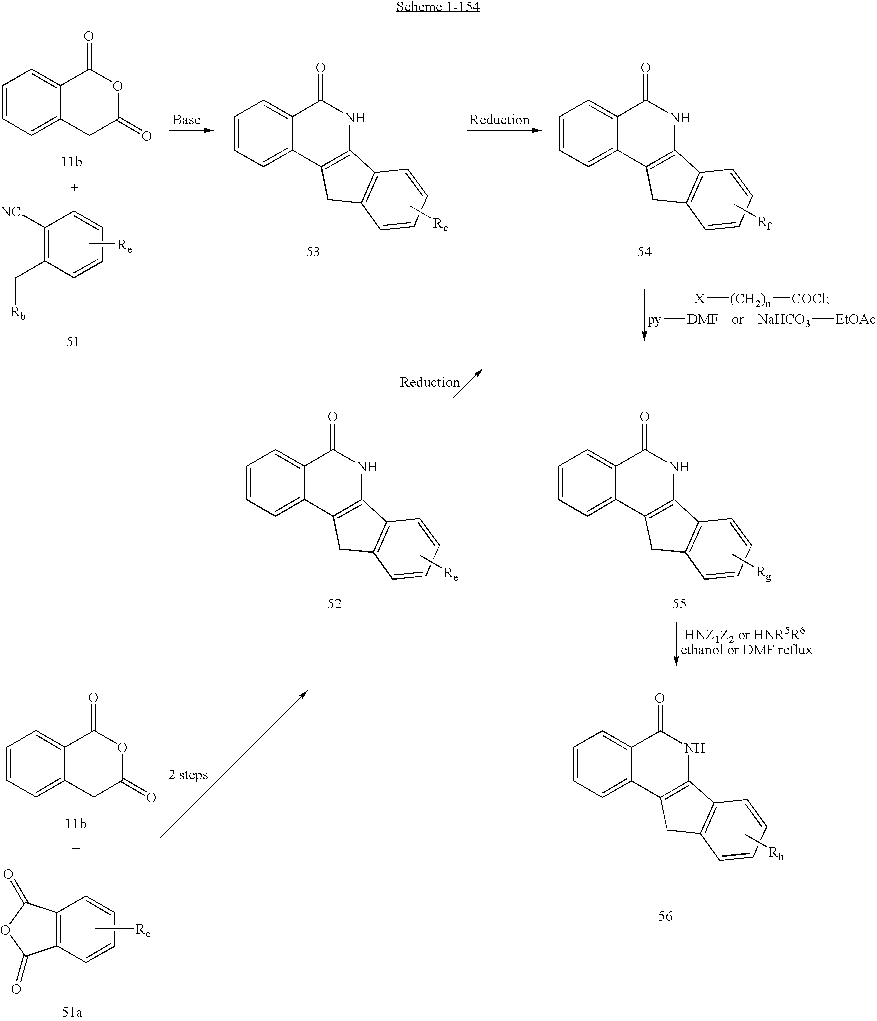
wherein: n is as defined above for Formula (I-154) and Formula (II-154);
R⁵ and R⁶ are as defined above for Formula (I-154);
Z₁ and Z₂ as defined above for Formula (II-154);
X is a leaving group such as bromide or chloride;
R_{b} is —Cl, —Br, —I, —OMs, —OTs, or —OTf;
Rₑ is —NO₂;
R_{f} is —NH₂;
R_{g} is —NHC(O)—(CH₂)ₙ—X; and
Rₕ is —NHC(O)—(CH₂), —NZ₁Z₂ or —NHC(O)—(CH₂)ₙ—N(R⁵)(R⁶).

In one embodiment, R_{b} is —Br.

General Procedure for the Preparation of Compounds of Formula 56

To a solution of homophthalic anhydride (11b) (about 1 equivalent) in a suitable solvent, such as acetonitrile, is added a compound of Formula 51 (about 1 to about 2 equivalents), follwed by a suitable base, such as triethylamine (about 1 to about 5 equivalents). The resultant reaction mixture is allowed to stir for about 1 hour, at which time a precipitate appears. The reaction mixture is then heated to reflux for about 20 hours, cooled to room temperature and filtered. The collected solid is washed with acetonitrile and dried under vacuum to provide a compound of Formula 53.

Compound 52 can be prepared from homophthalic anhydride (11b) and benzoic anhydride in two steps. Homophthalic anhydride and benzoic anhydride are reacted in a suitable solvent such as pyridine in the presence of an acid such as HCl, subsequently reacted with acetic anhydride in pyridine and heated to reflux, and then refluxed in the presence of an amine such as NH₃ in MeOH, to provide the compound of Formula 52.

To a solution of the compound of Formula 52 or 53 in a suitable solvent, such as DMF, is added a reducing agent, such as ammonium formate in the presence of palladium on carbon. The reaction mixture is heated to a temperature of about 90 to 100° C., cooled to room temperature and filtered to provide a compound of the Formula 54.

The compound of the Formula 54 can be reacted with X—(CH₂)ₙ—COCl, under conditions effective to form an amide of the Formula 55.

The compound of Formula 55 can be reacted with an amine of Formula HNZ₁Z₂, or an amine of Formula HNR⁵R⁶ in the presence of a solvent such as ethanol or DMF and heating to reflux, to form the compound of Formula 56.

### 4.17 Methods for Making the Compounds of Formulas (II-123), (IIa-123), and (VI-123)

Examples of synthetic pathways useful for making compounds of Formulas (II-123), (IIa-123), and (VI-123) are set forth in Example 5-123 below and generalized in Schemes 1-123 and 5-123.

Scheme 1-123 illustrates methods useful for making compounds of Formula (IIa-123), where R₁-R₄ and R₆-R₁₀ are defined for Formula (IIa-123). 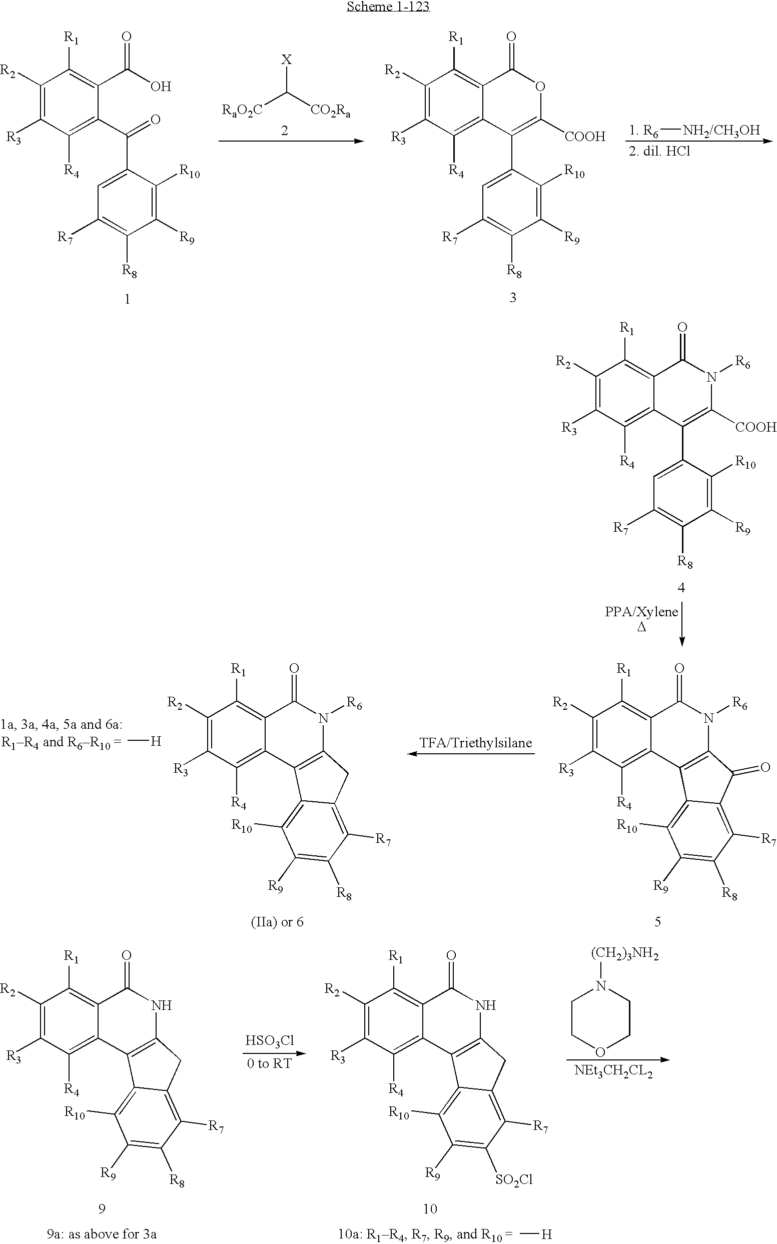 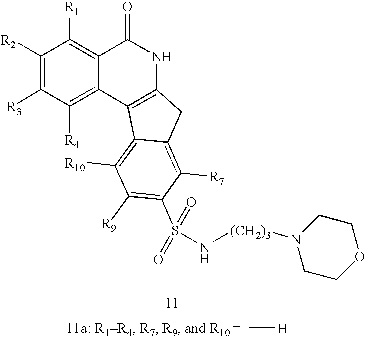
wherein
R₁-R₄ and R₆-R₁₀ are as defined above for the compounds of Formula (IIa-123);
each Rₐ is independently C₁-C₃ alkyl; and X is —Cl, —Br, —I, —OTf, —OMs or —OTs.

Compounds of Formula (II-123) and Formula (IIa-123) can be made by a method comprising the steps of Scheme 1-123 above herein.

A compound of Formula 3 can be made by a method comprising contacting a compound of Formula 1 with a compound of Formula 2 in the presence of a base for a time and at a temperature sufficient to make a compound of Formula 3.

In one embodiment Rₐ is methyl and X is —Br.

In one embodiment about 0.1 to about 10 equivalents of a compound of Formula 2 are used per about 1 equivalent of a compound of Formula 1.

In another embodiment about 0.5 to about 5 equivalents of a compound of Formula 2 are used per about 1 equivalent of a compound of Formula 1.

In still another embodiment, about 1 to about 2 equivalents of a compound of Formula 2 are used per about 1 equivalent of a compound of Formula 1.

In one embodiment about 1 to about 5 equivalents of base are used per about 1 equivalent of a compound of Formula 1.

In another embodiment about 2 to about 3 equivalents of base are used per about 1 equivalent of a compound of Formula 1.

Suitable bases for use in the method are organic bases such as triethylamine, diisopropylamine, diisopropylethylamine, pyridine, lutidine and imidazole; and inorganic bases such as alkali metal carbonates, including sodium carbonate, potassium carbonate and cesium carbonate.

In another embodiment, the base is potassium carbonate.

The method can be carried out in the presence of a solvent, such as acetonitrile, methylene chloride, chloroform, THF, DMF, DMSO, ethyl acetate, acetone, benzene, diethyl ether, water or mixtures thereof.

In another embodiment, the solvent is DMF.

In still another embodiment, the solvent is substantially anhydrous, i.e., comprises less than about 1% water.

In another embodiment the method is carried out for a time of about 2 hours to about 36 hours.

In still another embodiment the method of Scheme 1-123 is carried out for a time of about 8 hours to about 24 hours.

In yet another embodiment the method of Scheme 1-123 is carried out for a time of about 12 hours to about 18 hours.

In a further embodiment, the method of Scheme 1-123 is carried out at a temperature of about 0° C. to about 100° C.

In another embodiment, the method of Scheme 1-123 is carried out at a temperature of about 35° C. to about 70° C.

In yet another embodiment, the method of Scheme 1-123 is carried out at a temperature of about 25° C.

A compound of Formula 4 can be made by a method comprising (a) contacting a compound of Formula 3 with ammonia in methanol; and (b) contacting the product of step (a) with dilute acid for a time and at a temperature sufficient to make a compound of Formula 4.

In one embodiment about 1 to about 1000 equivalents of a solution of ammonia in methanol are used per about 1 equivalent of a compound of Formula 3.

In another embodiment about 5 to about 500 equivalents of ammonia in methanol are used per about 1 equivalent of a compound of Formula 3.

In still another embodiment, about 10 to about 100 equivalents of ammonia in methanol are used per about 1 equivalent of a compound of Formula 3.

In yet another embodiment about 20 to about 50 equivalents of ammonia in methanol are used per about 1 equivalent of a compound of Formula 3.

In one embodiment the ammonia in methanol is from about 1 N to about 10 N.

In another embodiment the ammonia in methanol is from about 3 N to about 7 N.

In one embodiment the dilute acid is from about 0.01 N to about 3 N.

In another embodiment the dilute acid is from about 0.1 N to about 1 N.

In another embodiment, the acid is HCl.

In one embodiment the method is carried out for a time of about 1 hour to about 48 hours.

In still another embodiment the method is carried out for a time of about 8 hours to about 36 hours.

In yet another embodiment the method is carried out for a time of about 12 hours to about 24 hours.

In one embodiment, the method is carried out at a temperature of about 0° C. to about

In another embodiment, the method is carried out at a temperature of about 25° C. to about 75° C.

In yet another embodiment, the method is carried out at a temperature of about 40° C. to about 60° C.

A compound of Formula 5 can be made by a method comprising contacting a compound of Formula 4 with a dehydrating agent for a time and at a temperature sufficient to make a compound of Formula 5.

In one embodiment about 0.1 to about 10 equivalents of a dehydrating agent are used per about 1 equivalent of a compound of Formula 4.

In another embodiment about 0.5 to about 5 equivalents of a dehydrating agent are used per about 1 equivalent of a compound of Formula 4.

In still another embodiment, about 1 to about 2 equivalents of a dehydrating agent are used per about 1 equivalent of a compound of Formula 4.

Suitable dehydrating agents include, but are not limited to, PPA, sulfuric acid, chlorosulfonic acid, sulfuryl chloride and thionyl chloride.

In another embodiment, the dehydrating agent is PPA.

The method can be carried out in the presence of a solvent, including, but not limited to, xylenes.

In one embodiment, the solvent is xylenes.

In another embodiment, the solvent is substantially anhydrous, i.e., comprises less than about 1% water.

In one embodiment the method is carried out for a time of about 1 hour to about 24 hours.

In still another embodiment the method is carried out for a time of about 4 hours to about 18 hours.

In yet another embodiment the method is carried out for a time of about 6 hours to about 12 hours.

In one embodiment, the method is carried out at a temperature of about 25° C. to about 200° C.

In another embodiment, the method is carried out at a temperature of about 100° C. to about 160° C.

A compound of Formula (IIa-123) can be made by a method comprising contacting a compound of Formula 5 with a reducing agent for a time (e.g. Wolff-Kishner reagents) and at a temperature sufficient to make a compound of Formula (IIa-123).

In one embodiment about 0.1 to about 10 equivalents of a reducing agent are used per about 1 equivalent of a compound of Formula 5.

In another embodiment about 0.5 to about 5 equivalents of a reducing agent are used per about 1 equivalent of a compound of Formula 5.

In still another embodiment, about 1 to about 2 equivalents of a reducing agent are used per about 1 equivalent of a compound of Formula 5.

Suitable reducing agents for this carbonyl reduction include, but are not limited to, sodium borohydride, diisobutylaluminum hydride, alpineborane, and TFA/triethylsilane.

In one embodiment, the reducing agent is a hydride reducing agent.

In another embodiment, the reducing agent is sodium borohydride.

In another embodiment, the reducing agent is TFA/triethylsilane.

The method can be carried out in the presence of a solvent, including, but not limited to, methanol, ethanol, THF and benzene. Alternatively the method can be carried out in the absence of a solvent.

In one embodiment, the solvent is methanol.

In another embodiment, the solvent is substantially anhydrous, i.e., comprises less than about 1% water.

In one embodiment the method is carried out for a time of about 1 minute to about 12 hours.

In still another embodiment the method is carried out for a time of about 5 minutes to about 6 hours.

In yet another embodiment the method is carried out for a time of about 15 minutes to about 2 hours.

In one embodiment, the method is carried out at a temperature of about −20° C. to about 40° C.

In another embodiment, the method is carried out at a temperature of about 10° C. to about 30° C.

In still another embodiment, the method is carried out at a temperature of about 25° C.

A compound of the Formula 9 can be further derivatized using methodology familiar to one skilled in the art of organic synthesis to prepared a variety of analogs of Formula (II-123) and Formula (IIa-123) having various substituents at one or more of R₁, R₂, R₃, R₄, R₇, R₈, R₉ and R₁₀. Useful derivatization methods include, but are not limited to, aromatic nucleophilic substitution reactions and aromatic electrophilic substitution reactions, such as nitration, iodination, bromination, chlorination, sulfonylation, sulfonylchlorination, alkylation and acylation. See M. B. Smith and J. March, Advanced Organic Chemistry: Reactions, Mechanisms, and Structure 675-758 and 850-893 (5^{th} ed. 2001).

In one embodiment, the compound of Formula 9 is transformed into the chlorosulfonyl compound of Formula 10 using chlorosulfonic acid. The Chlorosulfonyl compound of Formula 10 is then derivatized to the corresponding 3-(N morpholinyl)-propylsulfonamide derivative of Formula 11 by reacting the chlorosulfonyl compound of 10 with 3-(N-morpholinyl)-propylamine in the presence of a triethylamine.

Scheme 5-123 below illustrates methods useful for making compounds of Formula (II-123) and Formula (VI-123) wherein R₁-R₄, R₇-R₁₀, G₁-G₄, and X are defined above for the compounds of Formula (II-123) and Formula (VI-123):

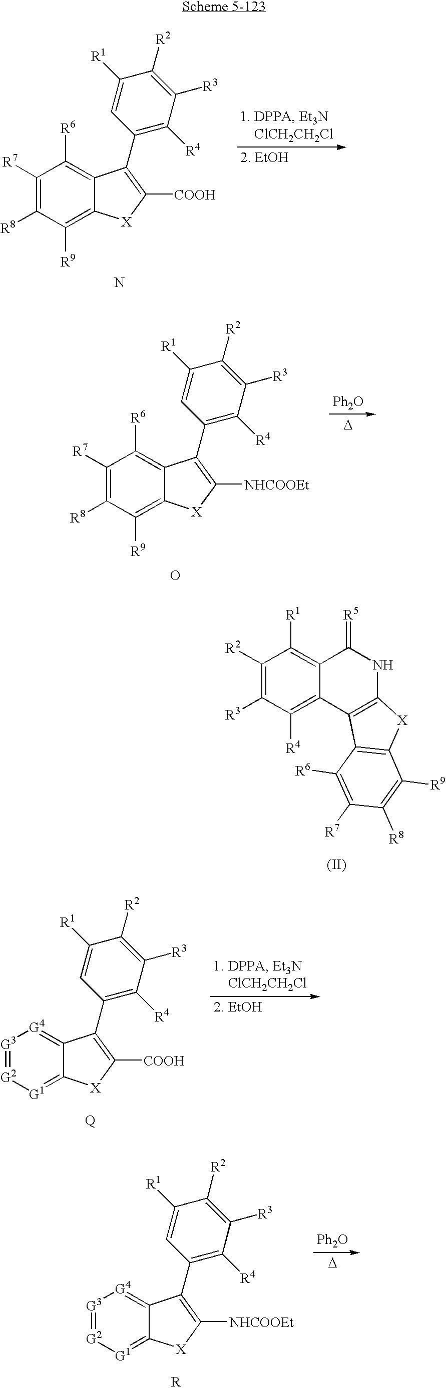

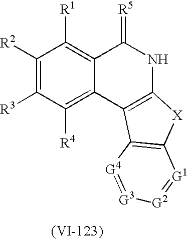

The carboxylic acid group of a compound of Formula N can be coupled with DPPA to provide the corresponding carbamate intermediates of Formula O, which can then be thermally cyclized to provide the compounds of Formula (II-123). Using the same synthetic method, the bicyclic carboxylic acids of Formula Q (see Wacker et al., Tet. Lett., 43:5189-5191, 2002; and Bourdais, et al., J. Het. Chem., 12:1111-1115, 1975, for methods useful to make compounds of Formula Q) can be converted to the compounds of Formula (VI-123) via the intermediacy of the carbamates of Formula R.

The invention is further described in the following examples, which do not limit the scope of the invention described in the claims.

### 5. EXAMPLES

Examples 1-149a-1-149hh relate to the synthesis of compounds of Formulas (I-149) and (IV-149) and refer to the compounds depicted in schemes 1-149-10-149.

Example 3-153 relates to the synthesis of compounds of Formula (I-153) and refers to the compounds depicted in scheme 1-153.

Examples 4-154a-4-154g relate to the synthesis of compounds of Formulas (I-154), and (II-154) and refer to the compounds depicted in scheme 1-154.

Examples 5-123a-5-123x relate to the synthesis of compounds of Formulas (II-123), (IIa-123), and (VI-123) and refer to the compounds depicted in schemes 1-123-6-123.

### 5.1 Example 1-149

### Preparation of Illustrative Compounds of Formula (I-149) or (IV-149)

### 1-149a) General Methods

Proton NMR spectra were obtained using a Varian 300 MHz spectrophotometer and chemical shift values (δ) are reported in parts per million (ppm). TLC was performed using TLC plates precoated with silica gel 60 F-254, and preparative TLC was performed using precoated Whatman 60A TLC plates. All intermediates and final compounds were characterized on the basis of ¹H NMR and MS data.

### 1-149b) Preparation of 5,6-dihydro-5,11-diketo-11H-indeno[1,2-c]isoquinoline (2)

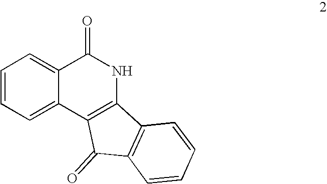

A stirred suspension of 1 (55 g, 0.22 mol) in NH₃/MeOH (7.0 N, 700 mL) was refluxed for 24 h. The reaction mixture was then allowed to cool to room temperature and was filtered and washed with MeOH to provide 46 g of the orange colored above-titled product in 84% yield. ¹H NMR (DMSO-d₆): δ 7.48-7.61 (m, 4H), 7.80-7.88 (m, 1H), 7.86 (d, J=8.7 Hz, 1H), 8.22 (d, J=8.4 Hz, 1H), 8.44 (d, J=7.5 Hz, 1H), 13.05 (s, 1H); ³C NMR (DMSO-D₆): δ 106.33, 121.63, 122.94, 123.27, 124.80, 128.45, 132.17, 133.60, 134.03, 134.68, 134.68, 134.81, 137.09, 156.41, 163.76, 190.57; MS (ES⁻): m/z 246.2 (M−1); Anal. Calcd for C₆H₉NO₂: C, 77.72; H, 3.67; N, 5.67; Found: C, 77.54; H, 3.69, N, 5.69.

### 1-149c) Preparation of (±) 11-hydroxy-5,6-dihydro-5-oxo-11H-indeno[1,2-c]isoquinoline (3a)

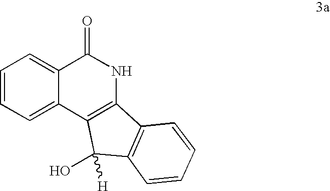

To a stirred suspension of 2 (2.5 g, 0.01 mol) in EtOH (25 mL) was added NaBH₄ (3.75 g, 0.1 mol) at room temperature in small portions over 30 min. The reaction mixture was stirred for an additional 2 h and then cooled to 0° C. It was then triturated with 10% HCl (10% soln.). The resulting solid precipitated was filtered and washed with water and MeOH to provide 3a (2.326 g, 92%). ¹H NMR (DMSO-d₆): δ 5.58 (d, J=8.1 Hz, 1H), 5.78 (d, J=8.7 Hz, 1H), 7.33-7.89 (m, 6H), 7.95 (d, J=7.8 Hz, 1H, 8.22 (d, J=7.8 Hz, 1H), 12.29 (s, 1H); ¹³C NMR (DMSO-d₆): S 77.44, 118.81, 120.15, 124.28, 125.04, 125.67, 126.34, 128.46, 128.64, 128.95, 133.27, 135.62, 136.12, 139.93, 148.55, 163.69; MS (ES⁺): m/z 250.1 (M+1); Anal. Calcd for C₆H₁₁NO₂: C, 77.10; H, 4.45; N, 5.62. Found: C, 77.01; H, 4.57, N, 5.59.

Similarly, by reacting 2 with MeMgI and m-MeO—C₆H₄MgBr, respectively, compounds (±) 11-hydroxy-11-methyl-5,6-dihydro-5-oxo-11H-indeno[1,2-c]isoquinoline (3b) and (±) 11-hydroxy-11-(2-methoxyphenyl)-5,6-dihydro-5-oxo-11H-indeno[1,2c]isoquinoline (3c) were prepared.

### 1-149d) Preparation of 11-substituted 5,6-dihydro-5-oxo-11H-indeno[1,2-c]isoquinolines (5a-e)

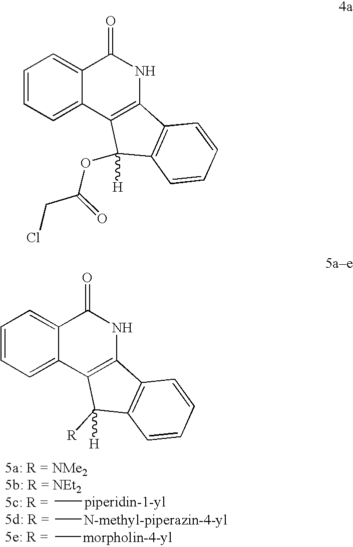

To a stirred suspension of 3a (0.5 g, 2 mmol) in pyridine (10 mL) was added chloroacetyl chloride (0.81 g, 0.006 mol) at 0° C. The reaction mixture was allowed to warm to room temperature and allowed to stir for 24 h. The reaction mixture was then poured on ice and extracted with EtOAc. The organic layer was separated, dried and concentrated to provide crude compound 4a, which was treated further with dimethylamine and stirred at room temperature for 24 h. The reaction mixture was poured on ice, and treated with 10% HCl The resulting mixture was then basified using saturated aqueous NaHCO₃ and the resulting solid was filtered to provide the desired product 5a. ¹H NMR (DMSO-D₆): δ 2.31 (s, 6H), 5.00 (s, 1H), 7.28-7.45 (m, 3H), 7.68-7.73 (m, 2H), 7.95 (d, J=6.9 Hz, 1H), 8.10 (d, J=7.8 Hz, 1H), 8.21 (d, J=8.1 Hz, 1H), 12.26 (s, 1H); ¹³C NMR (DMSO-D₆): δ 68.09, 116.28, 120.52, 124.58, 125.74, 126.27, 126.34, 127.68, 128.64, 133.02, 136.27, 144.45, 163.80; MS (ES⁺): m/z 277.2 (M+1).

The following compounds were also prepared by reacting 4a as above with diethylamine, piperidine, N-methylpiperidine and morpholine, respectively: (±) 11-diethylamino-5,6-dihydro-5-oxo-11H-indeno[1,2-c]isoquinoline (5b), (±) 11-piperizin-5,6-dihydro-5-oxo-11H-indeno[1,2-c]isoquinoline (5c), (±) 11-(N-methylpiperazin)-5,6-dihydro-5-oxo-11H-indeno[1,2-c]isoquinoline (5d), and (±) 11-morpholino-5,6-dihydro-5-oxo-11H-indeno[1,2-c]isoquinoline (5e).

### 1-149-e) Preparation of (±) 11-morpholino-5,6-dihydro-5-oxo-1H-indeno[1,2-c]isoquinoline (5e)

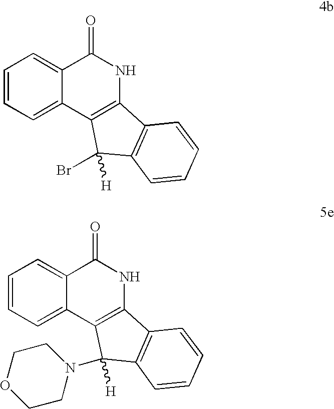

To a stirred suspension of 3a (0.6 g, 2.4 mmol) in trifluoroacetic acid (5 mL) was added phosphorus tribromide (1.0 M soln. in CH₂Cl₂, 3 mL) at room temperature, and the reaction mixture was stirred for 8 h. The reaction mixture was poured on ice and the resulting solid was filtered to provide bromo compound 4b (0.61 g, 76%). ¹H NMR (DMSO-d₆): δ 7.35-7.50 (m, 3H), 7.61 (d, J=6.6 Hz, 1H), 7.73-7.82 (m, 2H), 7.94 (d, J=6.6 Hz, 1H), 8.23 (d, J=7.8 Hz, 1H, 12.41 (s, 1H); ¹³NMR (DMSO-d₆): δ 52.06, 79.35, 114.43, 120.56, 123.58, 125.27, 125.50, 126.68, 128.55, 128.86, 129.66, 133.73, 135.91, 136.61, 141.39, 143.95, 163.74.

Compound 4b (0.5 g) was suspended in MeOH (10 mL) and treated with excess morpholine (about 5.0 eq) at room temperature and stirred at 60° C. for 3 h. The reaction mixture was poured on ice, and diluted with ethyl acetate (40 mL). The organic layer was separated and extracted in dil. HCl (10% soln.), the aqueous layer was then basified with sat. aq. NaHCO₃ and the resulting solid precipitated was filtered and dried to provide compound 5e (0.46 g, 90%). ¹H NMR (DMSO-d₆): δ 2.56 (m, 4H), 3.49 (m, 4H), 5.04 (s, 1H), 7.31-7.45 (m, 3H), 7.65-7.76 (m, 2H), 7.96 (d, J=7.2 Hz, 1H), 8.20-8.24 (m, 2H), 12.29 (s, 1H); ¹³C NMR (DMSO-D₆): δ 49.36, 67.62, 68.11, 115.20, 120.60, 124.47, 125.84, 126.34, 126.41, 127.76, 128.30, 128.72, 133.09, 136.30, 136.96, 140.35, 144.44, 163.67.

### 1-149f) Preparation of 5,6-dihydro-5-oxo-11H-indeno[1,2-c]isoquinoline (6)

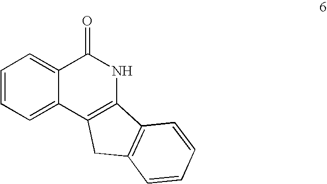

Method I: To a stirred solution of the alcohol 3a (0.35 g, 1.4 mmol) in trifluoroacetic acid (10 mL) was added at room temperature triethylsilane (0.812 g, 7 mmol) and the reaction mixture was stirred for 24 h. Trifluoroacetic acid was evaporated in vacuo and EtOAc was added to the resulting crude product. The resulting solid was filtered and washed with H₂O and EtOAc to provide the above-titled compound 6 (0.285 g, 87%). ¹H NMR (DMSO-D₆): δ 3.89 (s, 2H), 7.30-7.47 (m, 3H), 7.59 (d, J=6.9 Hz, 1H), 7.72-7.74 (m, 2H), 7.98 (d, J=7.8 Hz, 1H), 8.23 (d, J=8.4 Hz, 1H), 12.31 (s, 1H); ¹³C NMR(DMSO-d₆): δ 33.51, 116.50, 120.19, 124.01, 125.51, 125.55, 126.42, 127.50, 127.68, 128.56, 133.45, 136.39, 137.53, 140.18, 143.80, 163.46; MS (ES): m/z 232.1 (M−1); Anal. Calcd for C₁₆H₁₁NO: C, 82.38; H, 4.75; N, 6.00. Found: C, 81.79; H, 4.45, N, 5.99.

Method II: To a stirred suspension of 2 (40 g, 0.16 mol) in trifluoroacetic acid (2.5 L) was added triethylsilane (94 g, 0.8 mol) in small portions at room temperature and the reaction mixture was stirred for 96 h, during which time the reaction progress was monitored using TLC (eluent—5% MeOH/CH₂Cl₂). The reaction mixture was slowly poured on ice, filtered, washed with copious amounts of H₂O and MeOH and dried in vacuo to provide the above-titled compound 6 (33.1 g, 88%), whose spectral data were identical to those of a sample of compound 6 that was obtained using Method I.

### 1-149g) Preparation of 9-chlorosulfonyl-5,6-dihydro-5-oxo-11H-indeno[1,2-c]isoquinoline (7)

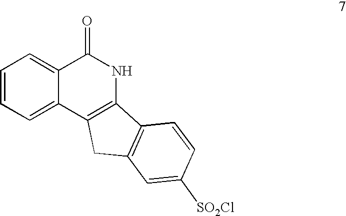

Compound 6 (40 g, 0.17 mol) was added in small portions to chlorosulfonic acid (112 mL, 1.71 mol) at 0° C. and the reaction mixture was allowed to warm to room temperature and allowed to stir for 2 h. The reaction mixture was slowly poured on ice and the resulting yellow solid was filtered, washed thoroughly with water and EtOAc and dried in vacuo to provide the above-titled product 7 (52 g, 92%). ¹H NMR (DMSO-d₆): δ 3.91 (s, 2H), 7.43-7.48 (m, 1H), 7.60 (d, J=7.2 Hz, 1H), 7.74-7.76 (m, 2H), 7.79 (s, 1H), 7.90 (d, J=7.5 Hz, 1H), 8.23 (d, J=7.8 Hz, 1H), Anal. Calcd. for C₁₆H₁₂ClNO₄S: C, 54.94; H, 3.46; N, 4.00. Found: C, 55.28; H, 3.43, N, 3.68, Karl-Fisher, 2.95.

### 1-149h) Preparation of 9-sulphonamido derivatives of 5,6-dihydro-5-oxo-11H-indeno[1,2-c]isoquinolines (8a-af)

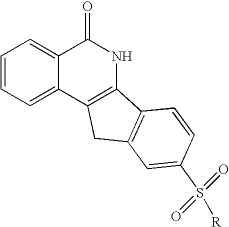 8a-af

a. R = 4-Methyl-piperazin-1-yl
b. R = 4-CH₂CO₂Me-piperazin-1-yl
c. R = 4-CH₂CO₂OH-piperazin-1-yl
d. R = imidazol-1-yl
e. R = L-prolinol
f. R = morpholin-4-yl
g. R = —NHCH₂CH₂NMe₂
h. R = —NHCH₂CH₂-pipendin-1-yl
i. R = —NHCH₂CH₂N-(pyridin-2-yl)
j. R = —NHCH₂CH₂-morpholin-4-yl
k. R = —NHCH₂CH₂-(2-N-Me-tetrahydropyrrolidin-1-yl)
l. R = —NHCH₂CH₂CH₂-morpholin-4-yl
m. R = —NHCH₂CH₂CH₂-(tetrahydropyrrolidin-1-yl)
n. R = —NHCH₂CH₂CH₂-imidazol-1-yl
a. R = —NHCH₂CH₂CH₂-(4-methylpiperazin-1-yl)
p. R = —N(CH₂CH₂NEt₂)₂
q. R = —N(CH₂CH₂NMe₂)₂
r. R = —N(CH₂CH₂OH)₂
s. R = —NHCH₂CH₂CN
t. R = —NHC(NH)NH₂
u. R = —NH[4-(1,2,4-triazole)]
v. R = —NH[4-(morpholin-4-yl)phenyl]
w. R = —NHCH₂CH₂(4-N-benzylpiperidine)
x. R = —NHCH₂CH₂(2-thienyl)
y. R = —NH[1-(4-azabenzimidazole)]
z. R = —NH[1-(4-(2′-pyridyl)piperazine)]
aa. R = —NHCH₂CH₂N[CH₂CH₂OH]₂
ab. R = —NH[1-(4-benzylpiperazine)]
ac. R = —NH₂
ad. R = —NHCH₂CH₂Ph
ae. R = —NHCH₂CH₂[4-OMe(phenyl)]
af. R =5 —NHC(O)(morpholin-4-yl)

Method I: To a stirred suspension of 3-(4-morpholino)-1-propylamine (17.28 g, 0.12 mol) in EtOAc was added sat. aq. NaHCO₃ (300 mL), and the mixture was allowed to stir for 15 min. Compound 7 (4.0 g, 0.012 mol) was then introduced in small portions at room temperature. The reaction mixture was stirred for 24 h; filtered and washed with H₂O, EtOAc and MeOH; refluxed in MeOH for 30 min; filtered while still warm; and washed with MeOH to provide compound 8l as a free base (2.33 g, 44%). ¹H NMR(DMSO-d₆): δ 1.47-1.52 (m, 2H), 2.16-2.21 (m, 4H), 2.47-2.48 (m, 2H), 3.44-3.48 (m, 2H), 3.23 (m, 4H), 4.02 (s, 2H), 7.49-7.58 (m, 1H), 7.78-7.82 (m, 3H), 7.97 (s, 1H), 8.14 (d, J=7.8 Hz, 1H), 8.26 (d, J=7.8 Hz, 1H), 9.59 (s, 1H), 12.42 (s, 1H).

The free bases of 8d, 8g, 8h, 8j, 8l, 8m-8r were also prepared by Method I, but substituting 3-(4-morpholino)-1-propylamine with imidazole, 2-dimethylamino-ethylamine, 2-(N-piperidinyl)-ethylamine, 2-(N-morpholinyl)-ethylamine, 3-(N-morpholinyl)propylamine, 3-(N-tetrahydropyrrolidinyl)-propylamine, 3-(N-imidazolyl)-propylamine, 3(N-(4-methylpiperazinyl)-propylamine, di-(2-(diethylamino)-ethyl)amine, di-(2(dimethylamino)-ethyl)amine and di-(2-hydroxyethyl)amine, respectively.

Method II: To a stirred suspension of 3-(4-morpholino)-1-propylamine (4.250 g) in CH₂Cl₂ (100 mL) was added 7 (1.950 g, 5.89 mmol) and the resulting mixture was stirred for 5 minutes. Subsequently, triethylamine (3 mL) was added and the reaction mixture was stirred for 24 h at room temperature. After this time the precipitate was collected and washed with MeOH (2×100 mL) and the crude solid product transferred to a round bottom flask. This material was diluted with MeOH (200 mL), heated to reflux for 30 min. and filtered while still warm. The resulting filtercake was washed with MeOH (200 mL) to provide the desired product as the free base of 8l (1.460 g, 56%).

The free bases of compounds 8a-r were prepared using Method II, but substituting 3(4-morpholino)-1-propylamine with about an equivalent amount of imidazole, 2-dimethylamino-ethylamine, 2-(N-piperidinyl)-ethylamine, 2-(N-morpholinyl)-ethylamine, 3(N-morpholinyl)-propylamine, 3-(N-tetrahydropyrrolidinyl)-propylamine, 3-(N-imidazolyl)propylamine, 3-(N-(4-methylpiperazinyl) propylamine, di-(2-(diethylamino)-ethyl)amine, di(2-(dimethylamino)-ethyl)amine and di-(2-hydroxyethyl)amine, respectively.

### 1-149k) Preparation of the methanesulfonate salt of 8l

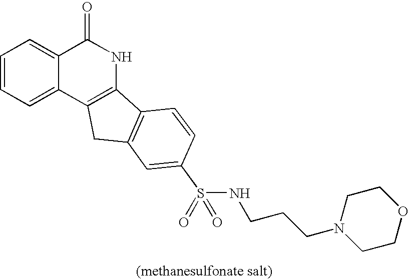

Free base 8l (1.0 g) was added to methanesulfonic acid (10 mL) at 0° C. and the resulting mixture was allowed to warm to room temperature and then stirred for 2 h. The reaction mixture was then poured into cold MeOH (100 mL, between −10° C. and 0° C.) and the precipitated solid was filtered, washed with MeOH (100 mL) and dried in vacuo. The dried solid was then dissolved in water (100 mL), filtered and lyophilized to provide the methanesulfonate monohydrate salt 8l. (1.020 g, 84%). ¹H NMR (DMSO-d₆): δ 1.75-1.85 (m, 2H), 2.35 (s, 3H), 2.78-2.84 (m, 2H), 2.96-3.12 (m, 4H), 3.36 (d, J=12.3 Hz, 2H), 3.61 (t, J=11.4 Hz, 2H), 3.94 (d, J=12.9 Hz, 2H), 4.03 (s, 2H), 7.49-7.55 (m, 1H), 7.76-7.84 (m, 3H), 7.99 (d, J=0.9 Hz, 1H), 8.15 (d, J=8.4 Hz, 1H), 8.25 (d, J=8.4 Hz, 1H), 9.59 (s, 1H), 12.42 (s, 1H); ¹³C NMR (DMSO-d₆): δ 24.27, 33.86, 51.89, 54.51, 64.02, 119.70, 120.39, 123.53, 126.09, 126.45, 128.63, 133.66, 135.80, 138.71, 141.21, 144.57, 163.29; Anal. Calcd for C₂₄H₃₁N₃O₄S₂: C, 52.06; H, 5.46; N, 7.59, Karl-Fisher, 3.36. Found: C, 51.85; H, 5.35, N, 7.30, Karl-Fisher, 4.32.

Similarly, HCl, H₂SO₄, CH₃COOH, and succinic acid salts of 8l were prepared by substituting methanesulfonic acid with about an equivalent amount of HCl, H₂SO₄ and CH₃COOH, respectively.

### 1-1491) Preparation of 5,6-dihydro-5-oxo-11H-indeno[1,2-c]isoquinoline (13a)

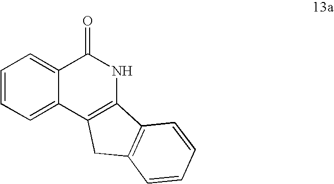

To a solution of homophthalic anhydride (324 mg, 2.0 mmol) in acetonitrile (15 mL) was added 2-cyanobenzyl bromide (431 mg, 2.0 mmol, 1.0 eq) and triethylamine (5 mL). The reaction was stirred under inert atmosphere at room temperature for 30 minutes, after which time a yellow precipitate appeared. The reaction mixture was then heated at reflux for 18 h and the resulting white precipitate was filtered, washed using acetonitrile (3×8 mL) and dried under vacuum to provide Compound 13a as a white crystalline solid. Yield=150 mg (32%).

### 1-149m) Preparation of α-Bromodimethylhomophthalate

Dimethylhomophthalate (83.1 g) was dissolved in dichloromethane (2 L) and N-bromosuccinimide (121 g, 1.7 eq) was added. The resulting suspension was irradiated for 18 h with a 500 Watt quartz-halogen lamp, which brought the reaction mixture to reflux. The reaction mixture was then washed sequentially with saturated aqueous sodium bicarbonate (4 L), saturated aqueous sodium bisulfite (2 L), and saturated aqueous sodium chloride (2 L). The organic phase was dried using sodium sulfate with a small amount of silica added to remove polar impurities. The organic phase was filtered and concentrated in vacuo to provide α-Bromodimethylhomophthalate as a dark orange oil. Yield=120.3 g (100%).

### 1-149n) Preparation of 8-Methoxy-6H-11-oxa-6-aza-benzo[a]fluoren-5-one

α-Bromodimethylhomophthalate (1.16 g) and 2-hydroxy-5-methoxybenzonitrile (0.6 g, 4 mmol, 1 eq) were dissolved by warming in acetonitrile (6 mL). Triethylamine (5.6 mL, 10 eq) was then added and the reaction was heated at reflux for 48 h under inert atmosphere, then cooled to room temperature. The reaction mixture was diluted with saturated sodium bicarbonate (40 mL) and the resulting suspension was allowed to stir for 2 h, and was then filtered. The filtercake was washed sequentially with 1 N HCl (2×50 mL), acetonitrile (2×50 mL) and dichloromethane (50 mL), then dried in a vacuum oven at 50° C. for three days to provide 8-Methoxy-6H-11-oxa-6-aza-benzo[a]fluoren-5-one as an white solid. Yield=0.81 g (76%).

### 1-149o) Preparation of 8-Hydroxy-6H-11-oxa-6-aza-benzo[a]fluoren-5-one

8-Methoxy-6H-11-oxa-6-aza-benzo[a]fluoren-5-one (5.0 g) was cooled using an ice bath, and boron tribromide (1 M in methylene chloride, 95 mL, 95 mmol, 5 eq) added in a steady stream under nitrogen. The reaction was heated at reflux under inert atmosphere for two hours, then cooled to room temperature and poured into water (150 mL). The resulting suspension was allowed to stir for 1 h, filtered, and the solids were washed with water (2×200 mL). The solids were then diluted with 5 N sodium hydroxide (600 mL) using heating. The resulting solution was cooled to 0° C. using an ice bath and the solution was acidified to pH 1 using conc. HCl. The resulting precipitate was vacuum filtered, and the solids washed sequentially with water (3×300 mL) and diethyl ether (300 mL) then dried overnight using a vacuum oven at 50° C. to provide 8-Methoxy-6H-11-oxa-6-aza-benzo[a]fluoren-5-one as a gray solid. Yield 4.74 g (100%).

### 1-149p) Preparation of 3-Nitroso-2-Phenyindole (28)

A solution of 2-phenylindole (27) (25 gm, 0.129 mol) in acetic acid (250 mL) was cooled to 18° C. and a solution of sodium nitrite (8 g, 0.115 mol) in water (10 mL) was added dropwise while keeping the temperature of the reaction at ca. 20° C. The resulting reaction was stirred for 30 min at room temperature then diluted with ice water (250 mL). The reaction mixture was was filtered and the solid was washed with water then recrystallized using methanol to provide Compound 28. Yield=27.5 gm (96.4%). ES-MS: 223.22 (M⁺+1); NMR (DMSO-d₆): δ 7.0 (m, 1H), 7.1 (m, 1H), 7.22 (m, 1H), 7.32 (m, 2H), 7.40 (m, 1H), 7.48 (m, 2H), 7.60 (m, 1H).

### 1-149q) Preparation of 3-Amino-2-Phenylindole (29)

To a solution of 3-nitroso-2-phenyl indole (28) (25 gm, 0.129 mol) in ethanol (450 ml) was added 2N sodium hydroxide (300 mL, 5.0 eq) followed by sodium dithionite (38 g). The reaction was heated at reflux for 5 h, then filtered. The solid was washed with water and dried under vacuum to provide Compound 29 as a yellow solid. Yield=15 g (72.1%). ES-MS: 209.25 (M⁺+1); NMR (DMSO-d₆): δ 7.0 (m, 1H), 7.1 (m, 1H), 7.22 (m, 1H), 7.32 (m, 2H), 7.40 (m, 1H), 7.48 (m, 2H), 7.60 (m, 1H).

### 1-149r) Preparation of 2-Phenylindole-3-ethylcarbamate (30)

To a 0° C. solution of 3-amino-2-phenylindole (29) (1.7 g, 8.17 mmol) in dichloromethane (150 ml) was added triethylamine (5 mL, 4.5 eq) followed by ethyl chloroformate (1 mL). The reaction was allowed to stir for 15 h, after which time the reaction mixture was diluted with water and transferred to a separatory funnel. The dichloromethane (50 mL), washed with water (2×50 mL), brine (50 mL) and dried over sodium sulfate. The solvent was removed and dried under vacuum to provide Compound 30 as a black solid (1.6 gm, 72.7%). ES-MS: 281.25 (M⁺+1); NMR (DMSO-d₆): δ 1.30 (t,3H), 4.12 (t, 2H), 7.0 (m, 1H), 7.1 (m, 1H), 7.22 (m, 2H), 7.32 (m, 2H), 7.40(m, 1H), 7.48 (m, 2H), 7.60 (m, 1H).

### 1-149s) Preparation of 6H,11H-Indolo[3,2-c]Isoquinoline-5-one (31)

A solution of 2-Phenylindole-3-aminoethylcarbamate (30) (1.4 g, 5 mmol) in diphenyl ether (10 ml) was heated at reflux for 4 h, then cooled to room temperature. The reaction mixture was filtered and the solid was washed sequentially using warm hexane and warm dichloromethane and dried under vacuum to provide Compound 31 as a gray solid. Yield=1.6 g (72.7%). ES-MS: 235.25 (M⁺+1); NMR (DMSO-d₆): δ 7.1 (t, 1H), 7.25 (t, 1H), 7.50 (m, 2H), 7.82 (t, 1H), 8.0 (d, I H), 8.14(d, 1H), 8.32 (t, 1H), 11.7(s, IH), 12.2 (s,1H).

### 1-149t) Preparation of 6H,11H-Indolo[3,2-c]Isoquinoline-5-one-9,11-diacetate (32)

To a 0° C. solution of 6H,11H-Indolo[3,2-c]Isoquinoline-5-one (31) (117 mg, 0.5 mmol) in dichloromethane (10 mL) was added triethylamine (2 mL, 30 eq) followed by acetic anhydride (1.8 mL, 35 eq). The reaction was stirred at room temperature for 48 h, then poured over ice and extracted with dichloromethane (100 mL). The dichloromethane layer was washed sequentially using water (2×20 mL) and brine (25 mL), then dried using sodium sulfate and concentrated in vacuo. The resulting solid residue was dried under vacuum to provide Compound 32 as a brown solid. Yield=180 mg, 83.7%. ES-MS: 430.57 (M⁺+1).

### 1-149u) Preparation of 6H,11H-Indolo[3,2-c]Isoquinoline-5-one-9,11-disulfonylchloride (33)

Compound 31 (117 mg, 0.5 mmol) was added to chlorosulfonic acid (2 mL, 60 eq) and the resulting reaction mixture was allowed to stir at room temperature for 4 hours, after which time the reaction mixture was poured over ice. The resulting precipitate was filtered, washed sequentially with water and ethyl acetate and dried under vacuum to provide Compound 33 as a light-yellow solid. Yield=180 mg (83.7%). ES-MS: 430.57 (M⁺+1); NMR (DMSO-d₆): δ 7.1 (t, 1H), 7.25 (t, 1H), 7.50 (m, 2H), 7.82 (t, 1H), 8.0 (d, 1H), 8.14(d, 1H), 8.32 (t, 1H), 11.7(s, 1H), 12.2 (s, 1H).

### 1-149v) Preparation of 6H,11H-Indolo[3,2-c]Isoquinoline-5-one-9,11-disulfonamide

To a solution of 33 (215 mg, 0.5 mmol) in methanol (10 mL) at 0° C. was added a 20% solution of ammonia in methanol (10 mL). The reaction mixture was allowed to stir at room temperature for 15 hours and was then filtered. The resulting solid was washed with methanol and the dried under vacuum to provide 6H,11H-Indolo[3,2-c]Isoquinoline-5-one-9,11-disulfonamide as a yellow solid. Yield=140 mg, 71.4%). ES-MS: 392.81 (M⁺+1).

### 1-149w) Preparation of N-acetylanthranilonitrile (36a)

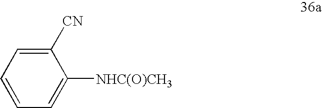

To a solution of anthranilonitrile (4.0 g, 32 mmol) in acetic anhydride (18 mL, 5.5 eq) at 90° C. was added 1 drop of sulfuric acid and the resulting reaction was stirred at 90° C. for 2 h, then allowed to sit at room temperature for 12 h. The reaction mixture was poured onto ice (ca. 200 mL) and the resulting solution was stirred for 2 h, after which time the solution was neutralized to pH 7.0 using 5 N sodium hydroxide. The resulting precipitate was filtered, washed using water (4×50 mL) and dried under vacuum for 72 h to provide Compound 36a as a white crystalline solid. Yield=1.07 g (16%).

### 1-149x) Preparation of 6H,11H-indolo[3,2-c]isoquinolin-5-one (37a)

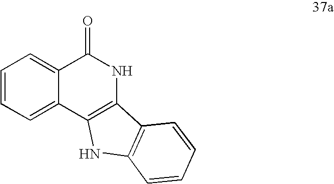

### From α-Bromodimethylhomophthalate

α-Bromodimethylhomophthalate (603 mg, 2.1 mmol) and N-acetylanthranilonitrile (36a) (370 mg, 1.1 eq) were dissolved in DMF (5 mL) under inert atmosphere. Potassium carbonate (1.45 g, 5.0 eq) was added and the reaction was stirred for 48 h at 100° C., then cooled to room temperature. The reaction mixture was poured into 1 N sodium hydroxide and the resulting mixture was extracted with EtOAc (50 mL). The EtOAc layer was washed sequentially with 1N HCl (50 mL), saturated aqueous sodium chloride (50 mL), dried over sodium sulfate, filtered and concentrated in vacuo. The resulting residue was dissolved by warming in toluene (70 mL) and the solution was cooled to room temperature and upon addition of hexanes (200 mL), a solid precipitate appeared. The solid precipitate was filtered, washed using hexanes (50 mL) and dried in a vacuum oven at 50° C. for 72 h to provide Compound 37a as a yellow powder. Yield=33 mg (6.7%).

### 1-149y) Preparation of 6H,11H-thia-6-aza-benzo[a]fluorene-5-one (40a)

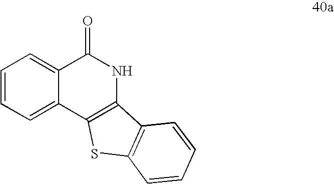

### From homophthalic anhydride

A solution of 2-mercaptobenzonitrile (1.35 g, 10 mmol) and homophthalic anhydride (1.6 g, 10.0 mmol, 1.0 eq) in acetonitrile (150 mL) under inert atmosphere was warmed with stirring until all reactants were in solution. Triethylamine (6.9 mL, 50 mmol, 5.0 eq) was added and the reaction was heated at reflux for 72 hours, then cooled to room temperature. After cooling, the reaction mixture was filtered, and the collected solid was washed using methanol (3×50 mL), then dried in a vacuum oven at 50° C. to provide Compound 40a as a white solid. Yield=225 mg (9%).

### From α-bromodimethylhomophthalate

A solution of 2-mercaptobenzonitrile (1.35 g, 10 mmol) and α-bromodimethylhomophthalate (2.87 g, 10.0 mmol, 1.0 eq) in acetonitrile (150 mL) under inert atmosphere was warmed with stirring until all reactants were in solution. Triethylamine (6.9 mL, 50 mmol, 5.0 eq) was added and the reaction was heated at reflux for 72 hours, then cooled to room temperature. After cooling, the reaction mixture was filtered, and the collected solid was washed using methanol (3×50 mL), then dried in a vacuum oven at 50° C. to provide Compound 40a as a white solid. Yield=250 mg (10%).

### 1-149z) Preparation of 5,6-Dihydro-5-oxo-9-nitro-indeno[1,2-c]isoquinoline (53a)

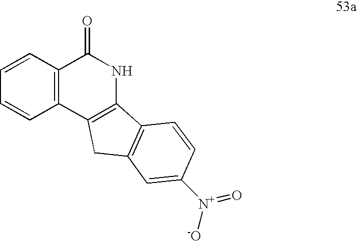

To a refluxing mixture of 2-methyl-4-nitro-benzonitrile (32.4 g, 0.2 mol) and NBS (44.470 g, 0.25 mol) in CCl₄ (300 ml) was added AIBN (0.325 g) and the resultant reaction mixture was refluxed for 4 hours. The reaction mixture was treated with AIBN (0.325 g, 31 mmol) and refluxed further for 4 hours. The reaction mixture was filtered, and the filtered succinimide was washed with CCl₄. The filtrate was concentrated in vacuo to provide a bromo compound (46 g). The bromo compound was dissolved in MeCN (200 ml), and to the reaction mixture was added homophthalic anhydride (30.780 g, 0.19 mol) at room temperature and under inert atmosphere. The reaction mixture was then treated with a solution of triethylamine (84 ml, 0.6 mol) in acetonitrile (100 ml). The reaction mixture was refluxed for 8 hours. The precipitate that formed was removed by filtration and washed with MeCN (100 ml). The washed precipitate was suspended in DMF (300 ml), which was heated at 130° C., then cooled and filtered. The resultant solid was washed with DMF (100 ml) and dried under vacuum to provide Compound 53a as a pale yellow solid (18.310 g, 33%). ¹H-NMR (DMSO-d₆): δ, 4.09 (s, 2H), 7.56 (m, 1H), 7.81-7.82 (m, 2H), 8.17 (d, J=8.4 Hz, 1H), 8.26-8.34 (m, 2H), 8.44 (s, 1H), 12.47 (s, 1H).

### 1-149aa) Preparation of 5,6-Dihydro-5-oxo-9-amino-indeno[1,2-c]isoquinoline (54a)

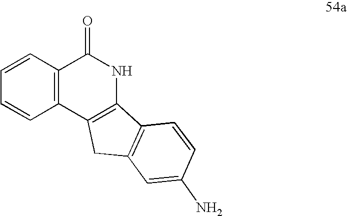

To a suspension of Compound 53a (5.3 g, 0.019 mol) and ammonium formate (5.985 g, 0.095 mol) in DMF (100 ml) was added Pd—C (5%, 100 mg) at 80° C. The reaction mixture was stirred at 100° C. for 1 hour. After the reaction mixture became clear, it was filtered through the pad of celite. The celite was washed with DMF. The filtrate was then diluted with ice, and the resultant solid was filtered, washed with water and dried at 80° C. under vacuum to provide Compound 54a (3.2 g, 68%). ¹H-NMR (DMSO-d₆): δ, 3.89 (s, 2H), 7.18 (d, J=8.4 Hz, 1H), 7.40-7.45 (m, 2H), 7.66-7.72 (m, 2H), 7.94 (d, J=8.1 Hz, 1H), 8.21 (d, J=8.1 Hz, 1H), 12.28 (s, 1H).

### 1-149bb) Preparation of N-[5,6-Dihydro-5-oxo-indeno[1,2-c]isoquinolin-9-yl]-4-bromo-butylamide (55a)

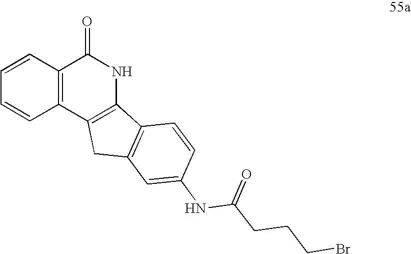

To a suspension of Compound 54a (1.5 g, 0.006 mol) in saturated NaHCO₃ (150 ml) and ethyl acetate (100 ml) was added 4-bromobutyryl chloride (5 eq). The reaction mixture was stirred at room temperature for 1 hour. The resultant solid was isolated by filtration, washed with water and ethyl acetate, and dried under vacuum to provide Compound 55a (1.625 g, 68%). ¹H-NMR (DMSO-d₆): δ, 2.09-2.13 (m, 2H), 2.47-2.52 (m, 2H), 3.58 (t, J=6.6 Hz, 2H), 3.85 (s, 2H), 7.40 (t, J=6.3 Hz, 1H), 7.50 (d, J=8.4 Hz, 1H), 7.66-7.71 (m, 2H), 7.86 (d, J=8.4 Hz, 1H), 7.92 (s, 1H), 8.20 (d, J=8.1 Hz, 1H), 10.10 (s, 1H), 12.24 (s, 1H).

### 1-149cc) Preparation of N-[5,6-Dihydro-5-oxo-indeno[1,2-c]isoquinolin-9-yl]4-chloro-butylamide (55b)

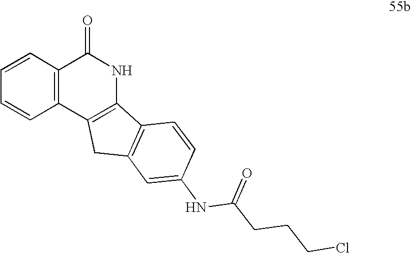

As set forth above for Compound 55a, Compound 55b (N-[5,6-dihydro-5-oxo-indeno[1,2-c]isoquinolin-9-yl]-4-chloro-butylamide) was prepared from the amino compound 54a using chlorobutyryl chloride in the presence of aqueous NaHCO₃ and ethyl acetate. ¹H-NMR (DMSO-d₆): δ, 1.99-2.08 (m, 2H), 2.47-2.52 (m, 2H), 3.70 (t, J=6.6 Hz, 2H), 3.86 (s, 2H), 7.38-7.44 (m, 1H), 7.50 (d, J=8.1 Hz, 1H), 7.66-7.71 (m, 2H), 7.86 (d, J=8.1 Hz, 1H), 7.95 (s, 1H), 8.21 (d, J=8.1 Hz, 1H), 10.09 (s, 1H), 12.24 (s, 1H).

### 1-149dd) Preparation of N-[5,6-Dihydro-5-oxo-indeno[1,2-c]isoquinolin-9-yl]-2-chloro-acetamide (55c)

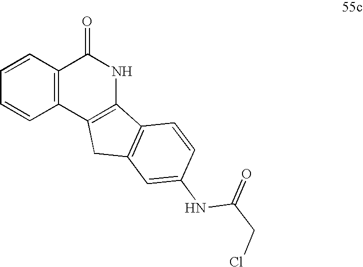

To a suspension of Compound 54a (1.5 g, 0.0060 mol) in saturated NaHCO₃ (250 ml) and ethyl acetate (250 ml) was added chloroacetyl chloride (5 eq). The reaction mixture was stirred at room temperature for 1 hour. The resultant solid was isolated by filtration; washed sequentially with ethyl acetate, water and methanol; and dried under vacuum to provide Compound 55c (1.6 g, 82%). ¹H-NMR (DMSO-d₆): δ, 3.89 (s, 2H), 4.27 (s, 2H), 7.40-7.45 (dd, J=6.3 and 8.1 Hz, 1H), 7.52 (d, J=8.1 Hz, 1H), 7.67-7.75 (m, 2H), 7.90 (d, J=8.4 Hz, 1H), 7.94 (s, 1H), 8.21 (d, J=8.1 Hz, 1H), 10.43 9s, 1H), 12.28 (s, 1H).

### 1-149ee) Preparation of N-[5,6-Dihydro-5-oxo-indeno[1,2-c]isoquinolin-9-yl]-4-morpholino-butylamide (73)

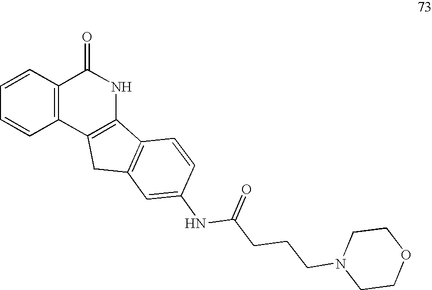

To a suspension of Compound 55a (1.625 g, 0.004 mol) in DMF (25 ml) was added triethyl amine (5 ml) followed by morpholine (5 ml). The reaction mixture was heated at 140 to 155° C. for 1 hour, cooled to room temperature and allowed to stir overnight. The resultant solid precipitate was filtered; washed sequentially with DMF, water and methanol; and dried under vacuum to provide the free base of Compound 73 (1.380 g, 85%). ¹H-NMR (DMSO-d₆): δ, 1.72-1.76 (dd, J=6.9 and 7.2 Hz, 2H), 2.26-2.37 (m, 8H), 3.51-3.54 (t, J=4.2 Hz, 4H), 3.86 (s, 2H), 7.39-7.43 (dd, J=6.3 and 6.6 Hz, 1H), 7.51 (d, J=6.6 Hz, 1H), 7.66-7.74 (m, 2H), 7.86 (d, J=8.4 Hz, 1H), 7.96 (s, 1H), 8.20 (d, J=8.1 Hz, 1H), 10.0 (s, 1H), 12.25 (s, 1H).

### 1-149ff) Preparation of the camphorsulfonic acid salt of 73

To a suspension of the Compound 73 (free base) (0.403 g, 0.001 mol) in MeOH (20 ml) was added camphor sulfonic acid (255 mg, 0.0011 mol). The reaction mixture was allowed to stir at room temperature for 2 hours. The reaction mixture was then concentrated in vacuo, and the resultant residue was dissolved in distilled, deionized water (40 ml); treated with decolorising charcoal (0.5 g); and stirred at 90 to 100° C. for 30 min. The resultant solution was filtered through the pad of celite, and the celite was washed with water. The filtrate was lyopholized to provide the camphorsulfonic acid salt of 73 (0.450 g, 71%). ¹H-NMR (DMSO-d₆): d, 0.72 (s, 3H), 1.02 (s, 3H), 1.20-1.30 (m, 2H), 1.76 (d, J=18 Hz, 1H), 1.82-1.86 (m, 1H), 1.89-1.97 (m, 3H), 1.99-2.25 (m, 1H), 2.35 (d, J=14.7 Hz, 1H), 2.43-2.48 (m, 2H), 2.64-2.71 (dd, J=11.7 and 14.7 Hz, 1H), 2.85 (d, J=14.7 Hz, 1H), 3.05-3.13 (m, 4H), 3.46 (d, J=11.7 Hz, 2H), 3.64 (t, J=12 Hz, 2H), 3.86 (s, 2H), 3.97 (d, J=12.3 Hz, 2H), 7.39-7.44 (dd, J=7.8 and 8.1 Hz, 1H), 7.52 (d, J=8.1 Hz, 1H), 7.67-7.75 (m, 2H), 7.87 (d, J=8.1 Hz, 1H), 7.96 (s, 1H), 8.21 (d, J=8.1 Hz, 1H), 9.57 (s, 1H), 10.15 (s, 1H), 12.25 (s, 1H).

### 1-149gg) Preparation of 2-Dimethylamino-N-(5,6-Dihydro-5-oxo-indeno[1,2-c]isoquinolin-9-yl)-acetamide (43)

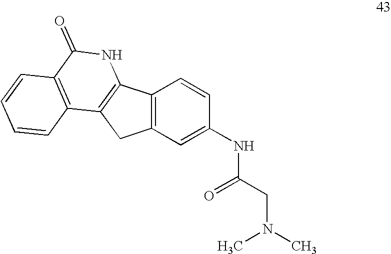

A suspension of Compound 55c (1.6 g, 0.0049 mol) and dimethyl amine in ethanol (2N, 200 ml) was refluxed for 24 h. Additional solution of dimethyl amine in ethanol (2N, 200 ml) was added. The reaction mixture was refluxed further for 24 hours and allowed to cool to room temperature. The resultant solid was filtered, washed with ethanol, and dried under vacuum to provide Compound 43 (1.510 g, 92%). ¹H-NMR (DMSO-d₆): δ, 2.27 (s, 6H), 3.07 (s, 2H), 3.85 (s, 2H), 7.38-7.43 (m, 1H), 7.58 (d, J=8.1 Hz, 1H), 7.66-7.73 (m, 2H), 7.87 (d, J=8.1 Hz, 1H), 8.02 (s, 1H), 8.20 (d, J=8.1 Hz, 1H), 9.82 (s, 1H), 12.21 (s, 1H); MS (ES⁺): m/z 334.01 (M+1).

### 1-149hh) Preparation of camphorsulfonic acid salt of 43

To a suspension of Compound 43 (free base) (0.1.250 g, 0.0037 mol) in MeOH (200 ml) was added camphor sulfonic acid (0.915 g, 0.0039 mol). The reaction mixture was allowed to at room temperature for 1 hour, and concentrated in vacuo. The resultant residue was dissolved in distilled, deionized water (300 ml); filtered; treated with decolorising charcoal (1 g); and allowed to stir at 100 to 105° C. for 30 minutes. The resultant solution was filtered through a pad of celite, and the celite was washed with water. The filtrate was lyophilized to provide the camphor sulfonic acid salt of Compound 43 (1.660 g, 75%). ¹H-NMR (DMSO-d₆): δ, 0.72 (s, 3H), 1.02 9s, 3H), 1.20-1.30 (m, 2H), 1.74-1.92 (m, 3H), 2.17-2.25 (m, 1H), 2.35 (d, J=14.7 Hz, 1H), 2.64 (t, J=9.9 Hz, 1H, 2.80 (d, J=14.7 Hz, 1H), 3.90 (s, 2H), 4.16 (s, 2H), 7.41-7.46 (dd, J=6.3 and 8.1 hz, 1H), 7.53 (d, J=8.1 Hz, 1H), 7.68-7.73 (m, 2H), 7.92-7.94 (m, 2H), 8.22 (d, J=8.1 Hz, 1H), 9.77 (s, 1H), 10.68 (s, 1H), 12.29 (s, 1H).

### 5.2 Example 3-153

### Preparation of Illustrative Compounds of Formula (I-153)

### 3-153a) Preparation of 3-Morpholin-4-yl-propane-1-sulfonic acid (5-oxo-5,11-dihydro-6H-indeno[1,2-c]isoquinolin-9-yl)-amide (6c)

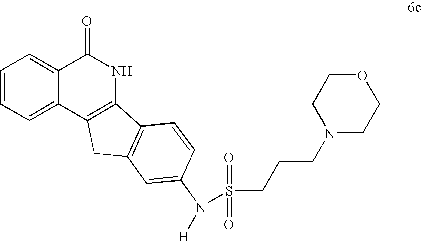

### Step A—Preparation of 3-morpholin-4-yl-propane-1-sulfonic acid (5-oxo-5,11-dihydro-6H-indeno[1,2-c]isoquinolin-9-yl)-amide

9-Amino-6H,11H-indeno[1,2-c]isoquinolin-5-one (250 mg) was diluted with DMF (10 ml) and to the resultant suspension is added triethylamine (3 eq), followed by 3-chloropropanesulfonyl chloride (2 eq) and the resultant reaction was allowd to stir at room temperature for about 1 hour. The reaction mixture was concentrated in vacuo and the resultant residue was diluted with methylene chloride (10 mL) to provide a solution from which a solid separated out. The solution was filtered and the collected solid was washed sequentially using ethyl acetate (10 mL), water (10 mL), and methanol (10 mL) to provide 3-morpholin-4-yl-propane-1-sulfonic acid (5-oxo-5,11-dihydro-6H-indeno[1,2-c]isoquinolin-9-yl)-amide, which was used without further purification. Yield=228 mg. ¹H-NMR (DMSO-D₆): 2.36-2.41 (m, 2H), 3.51 (t, J=7.5 Hz, 2H), 3.78 (t, J=6.3 Hz, 2H), 3.88 (s, 2H), 7.18 (d, J=6.6 Hz, 1H), 7.39-7.47 (m, 2H), 7.66-7.74 (m, 2H), 7.95 (d, J=8.4 Hz, 1H), 8.20 (d, J=8.1 Hz, 1H), 12.26 (s, 1H).

### Step B—Preparation of Compound 6c

3-Morpholin-4-yl-propane-1-sulfonic acid (5-oxo-5,11-dihydro-6H-indeno[1,2-c]isoquinolin-9-yl)-amide (220 mg, prepared using the method of Step A) was diluted with DMF (15 ml) and to the resultant suspension was added triethylamine (1 eq.) followed by morpholine (2 ml) and the reaction mixture was heated to reflux and allowed to stir for about 5 days. The reaction mixture was concentrated in vacuo and the resultant residue was diluted with methanol (10 mL) to provide a solution from which a solid separated out. The solution was filtered and the collected solid was washed sequentially using ethyl acetate (10 mL), water (10 mL), and methanol (10 mL) to provide Compound 6c (135 mg). MS: m/z 340 (M+1).

### 5.3 Example 4-154

### Preparation of Illustrative Compounds of Formula (_-154) or (II-154)

### 4-154a) Preparation of 2-[4-(4-Fluoro-phenyl)-piperazin-1-yl]-N-(5-oxo-5,11-dihydro-6H-indeno[1,2-c]isoquinolin-9-yl)-acetamide (11a)

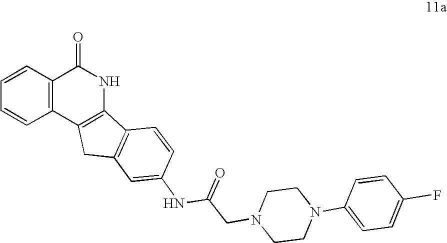

A suspension of Compound 55c (as prepared in Example 1-149 cc) (100 mg) and 4-(p-F-phenyl)piperazine in methanol (10 ml) was refluxed for overnight. The reaction mixture was allowed to cool to room temperature. The resultant solid was filtered, washed with methanol, and dried under vacuum to provide Compound 11a (120 mg).

### 4-154b) Preparation of N-(5-Oxo-5,11-dihydro-6H-indeno[1,2-c]isoquinolin-9-yl)-2-(4-phenyl-3,6-dihydro-2H-pyridin-1-yl)-acetamide (12a)

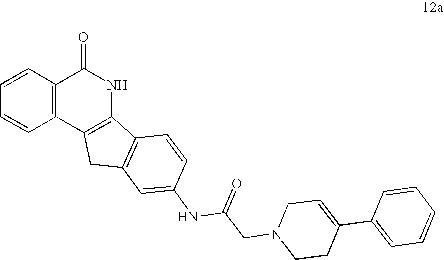

A suspension of Compound 55c (as prepared in Example 1-149 cc) (85 mg), triethylamine (1.2 eq), and 4-(phenyl)-1,2,5,6-tetrahydropyridine (62 mg) in DMF (15 ml) was heated at 60° C. for 16 hr. The reaction mixture was allowed to cool to room temperature. The resultant solid was filtered, washed with methanol, and dried under vacuum to provide Compound 12a (85 mg).

### 5.4 Example 5-123

### Preparation of Illustrative Compounds of Formula (II-123), (IIa-123), or (VI-123)

### 5-123a) Preparation of 4-Phenyl-3-isocoumarincarboxylic acid (3a)

Following the procedure described in Natsugary et al., J. Med. Chem. 38, 3106-3120 (1995), compound 3a (Scheme 1-123) was synthesized. A suspension of 1a (33.9 g, 0.15 mol) (Scheme 1-123), potassium carbonate (41.4 g, 0.3 mol) and diethyl bromomalonate (28.17 mL, 0.165 mol) in DMF (250 mL) was stirred at room temperature for 15 h. The reaction mixture was then diluted using cold water and extracted into ethyl acetate. The ethyl acetate layer was dried over sodium sulfate, and concentrated in vacuo to afford a crude residue to which was added glacial acetic acid (1.0 L) and concentrated HCl (800 mL). The resulting solution was heated at reflux for 6 h. The reaction mixture was cooled to room temperature and poured on ice water. The solid precipitate was filtered, washed with water and dried using vacuum to provide compound 3a as a white solid. Yield=32.6 g (84%).

### 5-123b) Preparation of 4-Phenyl-3-isoquinolinonecarboxylic acid (4a)(Scheme 1-123)

A stirred suspension of 3a (1.4 g, 0.0052 mol) in ammonia-methanol (7 N, 125 mL) was heated at reflux for 23 h, then cooled to room temperature. The reaction mixture was concentrated in vacuo, and the residue obtained was acidified with 10% aqueous HCl. The resulting solid was filtered, washed with water and dried under vacuum to provide compound 4a. Yield=1.225 g (89%).

### 5-123c) Preparation of 6H, 7-oxoindeno[2,1-c]isoquinolinone (5a) (Scheme 1-123)

### Using PPA

To a stirred suspension of compound 4a (0.225 g, 0.85 mmol) in xylenes (20 mL) was added polyphosphoric acid (0.600 g). The resulting reaction mixture was heated at reflux for 6 h. The reaction mixture was cooled to room temperature and concentrated in vacuo to provide a crude residue, which was poured on ice. The resulting solid was filtered, washed with water, and dried under vacuum to provide compound 5a. Yield=155 mg (74%).

### Using chlorosulfonic acid

Similarly, compound 4a (500 mg, 0.0019 mol) was suspended up in chlorosulfonic acid (2.5 mL) at 0° C. for 5 min, and the reaction mixture was stirred at room temperature for 5 min. After the reaction mixture became homogeneous, it was slowly poured on ice. The resultant red-colored solid precipitate was filtered, washed with water, and dried to provide compound 5a. Yield=395 mg (85%).

### 5-123d) Reduction of (5a) to 6H,7H-Hydroxyindeno[2,1-c]isoquinolin-5-one (6a) (Compounds of Formula IIa) (Scheme 1-123)

To a stirred suspension of 5a (1.0 g, 4.0 mmol) in methanol (25 mL) was slowly added solid sodium borohydride (385 mg) at room temperature. The resulting reaction mixture was stirred for 15 min. The reaction mixture was then poured on an ice-cold 1 N HCl solution, and the resulting solid was filtered, washed with water, and dried under vacuum to provide compound 6a (Formula IIa). Yield=0.940 g (93%).

### 5-123e) Preparation of 5-Oxo-5,7-dihydro-6H-indeno[2,1-c]isoquinoline-9-sulfonyl chloride (10a) (Scheme 1-123)

Compound 9a (R₁-R₄ and R₇-R₁₀═H) (210 mg, 0.9 mmol) was slowly added to a solution of chlorosulphonic acid (2.0 mL) at 0° C. for 5 min, and at room temperature for 5 min. After the reaction mixture became homogeneous, it was slowly poured on ice. The solid that precipitated was filtered, washed with water, and dried to provide compound 10a (180 mg, 60%).

### 5-123f) 5-Oxo-5,7-dihydro-6H-indeno[2,1-c]isoquinoline-9-sulfonic acid (3-morpholin-4-yl-propyl)-amide (11a) (Scheme 1-123)

A suspension of 10a (110 mg, 0.33 mmol) in methylene chloride (10 mL) was treated with 4-(3-morpholino)-1-propylamine (240 mg, 1.66 mmol) and triethylamine (1 eq), and the reaction mixture was stirred at room temperature for 1 h. The resulting mixture was diluted with ethyl acetate, and the solid that precipitated was filtered, washed with ether and dried to provide compound 11a. Yield=65 mg (45%).

### Example 2-149

### Compound 8l Methanesulfonate Salt is Useful for Treating or Preventing Erectile Dysfunction

Experiments were conducted in male Sprague-Dawley rats according to previously published methods for forceps-induced nerve crush injury and erectile function measurements (Rehman, J., et al., Urology 51:640-644, 1998; Sezen, S. F., et al., Int J. Impot. Res. 14:506-12, 2002). Subjects were anesthetized with Phenobarbital. The prostate of the subjects was exposed and the cavernosal nerve was clipped on either side with a forceps to induce mechanical injury (crush). This rat model mimics the nerve injury that develops during human male prostatectomy, leading to nerve injury and subsequent erectile dysfunction. Subjects were studied 2 weeks after the injury. Two groups of subjects were used, one group treated with vehicle and one group treated with compound 8l methanesulfonate salt. Compound 8l methanesulfonate salt was injected at 30 mg/kg i.v. immediately before the crush injury, and on the following day at the same dose. Thereafter, for 12 days, subjects were treated with 60 mg/kg compound 8l methanesulfonate salt intraperitoneally. At 2 weeks, subjects were re-anesthetized and measured for mean arterial blood pressure (MAP) and intracavernosal pressure (ICP). Cavernosal nerve stimulation was conducted at 5 and 7.5 V using a square pulse stimulator for 30 msec. ICP was determined as the area under curve (mmHg×sec). In addition, IPC/MAP ratios were determined. The results shown in Table 1 demonstrate that compound 8l methanesulfonate salt, an illustrative compound of the invention, improves erectile function in a rat model that mimics the nerve injury that develops during human male prostatectomy.

TABLE 1
ICP values and ICP/MAP ratios in vehicle treated and compound of the
invention-treated (compound 8l methanesulfonate salt) rats in response to
cavernous nerve crush injury (mean ± SEM). Normal range of ICP is
approximately 4000 mmHg × sec, and normal range of IPC/MAP
values are approx. 0.8-0.9 in normal uninjured animals.

| | | Compound 8l |
|---|---|---|
| | Vehicle | methanesulfonate salt |
| ICP | 1124 ± 212 | 1471 ± 151* |
| ICP/MAP | 0.16 ± 0.03 | 0.23 ± 0.02* |

*p < 0.05, n = 14-16.

Accordingly, Compound 8l (methanesulfonate salt), an illustrative compound of the invention, is useful for treating or preventing erectile dysfunction in a subject.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparant to those skilled in the art and are intended to fall within the scope of the appended claims.

A number of references have been cited, the entire disclosures of which have been incorporated herein in their entirety.

What is claimed is:

1. A method for treating or preventing erectile dysfunction, comprising administering to a subject in need thereof an effective amount of a compound having the Formula (I-149): 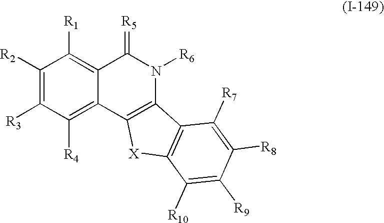
or a pharmaceutically acceptable salt thereof,
wherein:
R₅ is O, NH or S;
R₆ is —H or —C₁-C₅ alkyl;
X is —C(O)—, —CH₂—, —CH(halo)-, —CH(OH)—(CH₂)ₙ—, —CH(OH)—, —CH(-aryl)-, —O—, —NH—, —S—, —CH(NR₁₁R₁₂)— or —N(SO₂Y)—, wherein Y is —OH, —NH₂ or —(C₁-C₅ alkyl)-(3- to 7-membered monocyclic heterocycle);
R₁₁ and R₁₂ are independently -hydrogen or —C₁-C₁₀ alkyl; or N, R₁₁ and R₁₂ are taken together to form a -(nitrogen-containing 3- to 7-membered monocyclic heterocycle);
R₁ is -hydrogen, -halo, —C₁-C₁₀ alkyl, -(halo-substituted C₁-C₅ alkyl), —C₂-C₁₀ alkenyl, —(C₃-C₈ monocyclic cycloalkyl), -aryl, —NH₂, -(amino-substituted C₁-C₅ alkyl), —C(O)OH, —C(O)O(C₁-C₅ alkyl), —NO₂ or -A-B;
A is —SO₂—, —SO₂NH—, —NHC(O)—, —NHC(O)NH—, —O—, —C(O)—, —OC(O)—, —C(O)O—, —C(O)NH—, —C(O)N(C₁-C₅ alkyl)-, —NH—, —CH₂—, —S— or —C(S)—;
B is —C₁-C₁₀ alkyl, —C₂-C₁₀ alkenyl, -(3- to 7-membered monocyclic heterocycle), -(7- to 10-membered bicyclic heterocycle), —(C₃-C₈ monocyclic cycloalkyl), -aryl, —NZ₁Z₂, —(C₁-C₅ alkylene)-NZ₁Z₂, -(amino-substituted C₁-C₅ alkyl), —C₁-C₅ alkylene)-(3- to 7membered monocyclic heterocycle), —(H₂NC(O)-substituted aryl), —C(O)OH, —C(O)O—(C₁-C₅ alkyl), —(C₁-C₅ alkylene)-C(O)OH, —C(O)O-phenyl or —C(NH)NH₂, each of which, other than —NZ₁Z₂, —C(O)OH, and —C(NH)NH₂, is unsubstituted or substituted with one or more of -(hydroxy-substituted C₁-C₅ alkyl), -(amino-substituted C₁-C₅ alkyl), —O—(C₁-C₅ alkyl), -halo, -hydroxy, —NO₂, —CN, —NZ₁Z₂, -(nitrogen-containing 3- to 7-membered monocyclic heterocycle), -(nitrogen containing 7- to 10-membered bicyclic heterocycle), —C₁-C₁₀ alkyl, —C₂-C₁₀ alkenyl, —C₂-C₁₀ alkynyl, -aryl, -benzyl, —C(O)OH, —(C₁-C₅ alkylene)-C(O)O—(C₁-C₅ alkyl), —C₁-C₅ alkylene)-OC(O)—(C₁-C₅ alkyl), or —(C₁-C₅ alkylene)-C(O)OH, each of which is unsubstituted or substituted with —C₁-C₁₀ alkyl or -(hydroxy-substituted C₁-C₅ alkyl);
R₂, R₃, R₄, R₇, R₈, R₉ and R₁₀ are independently -(hydrogen, -halo, -hydroxy, —O—(C₁-C₅ alkyl), —C₁-C₁₀ alkyl, -(halo-substituted C₁-C₅ alkyl), —C₂-C₁₀ alkenyl, —(C₃-C₈ monocyclic cycloalkyl), -aryl, —NH₂, -(amino-substituted C₁-C₅ alkyl), —C(O)OH, —C(O)NH₂, —C(O)O(C₁-C₅ alkyl), —OC(O)(C₁-C₅ alkyl), —NO₂ or -A-B;
Z₁ and Z₂ are independently -hydrogen or —C₁-C₁₀ alkyl, which is unsubstituted or substituted with one or more of -halo, —OH or —NZ₃Z₄, where Z₃ and Z₄ are independently, -hydrogen or —C₁-C₅ alkyl, which is unsubstituted or substituted with one or more of -halo, -hydroxy, benzyl, or —NH₂; or N, Z₃ and Z₄ are taken together to form a -(nitrogen-containing 3- to 7-membered monocyclic heterocycle); or N, Z₁ and Z₂ are taken together to form a -(nitrogen-containing 3- to 7-membered monocyclic heterocycle); and
n is an integer ranging from 0-5.

2. The method of claim 1, wherein the compound has the following formula: 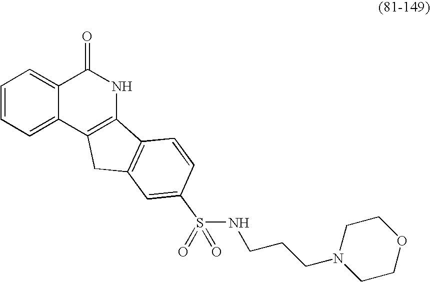
or a pharmaceutically acceptable salt thereof.

3. The method of claim 2, wherein the pharmaceutically acceptable salt is the methanesulfonate salt of (8l-149).

4. A method for treating or preventing erectile dysfunction, comprising administering to a subject in need thereof an effective amount of a compound having the Formula (IV-149): 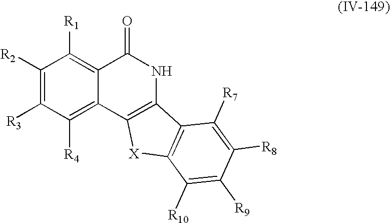
or a pharmaceutically acceptable salt thereof,
wherein:
X is —CH₂—, —O—, —NH—, or —S—;
R₁, R₂, R₃, R₄, R₇, R₉, R₉ and R₁₀ are independently -hydrogen, -halo, -hydroxy, —O—(C₁-C₅ alkyl), —C₁-C₁₀ alkyl, -(halo-substituted C₁-C₅ alkyl), —C₂-C₁₀ alkenyl, —(C₃-C₈ monocyclic cycloalkyl), -aryl, —NH₂, -(amino-substituted C₁-C₅ alkyl), —C(O)OH, —C(O)O(C₁-C₅ alkyl), —OC(O)(C₁-C₅ alkyl), —NO₂ or -A-B;
A is —SO₂—, —SO₂NH—, —NHC(O)—, —NHC(O)NH—, —O—, —CO—, —OC(O)—, —C(O)O—, —C(O)NH—, —C(O)N(C₁-C₅ alkyl)-, —NH—, —CH₂—, —S— or —C(S)—;
B is —C₁-C₁₀ alkyl, —C₂-C₁₀ alkenyl, -(3- to 7-membered monocyclic heterocycle), -(7- to 10-membered bicyclic heterocycle), —(C₃-C₈ monocyclic cycloalkyl), -aryl, —NZ₁Z₂, —(C₁-C₅ alkylene)-NZ₁Z₂, -(amino-substituted C₁-C₅ alkyl), —(C₁-C₅ alkyl)-(3- to 7-membered monocyclic heterocycle), —(H₂NC(O)-substituted aryl), —C(O)OH, —C(O)O—(C₁-C₅ alkyl), —C(O)O-phenyl or —C(NH)NH₂, each of which, other than —NZ₁Z₂, —C(O)OH, or —C(NH)NH₂, is unsubstituted or substituted with one or more of —O—(C₁-C₅ alkyl), -halo, -hydroxy, —NO₂, —CN, —NZ₁Z₂, -(nitrogen-containing 3- to 7-membered monocyclic heterocycle), -C₁-C₁₀ alkyl, —C₂-C₁₀ alkenyl, —C₂-C₁₀ alkynyl, -aryl, -benzyl, —C(O)OH, —(C₁-C₅ alkylene)-C(O)O—(C₁-C₅ alkyl) or —(C₁-C₅ alkylene)-OC(O)—(C₁-C₅ alkyl); and
Z₁ and Z₂ are independently —H or —C₁-C₁₀ alkyl, which is unsubstituted or substituted with one or more of -halo, —OH or —NZ₃Z₄, where Z₃ and Z₄ are independently, —H or —C₁-C₅ alkyl, which is unsubstituted or substituted with one or more of -halo, -hydroxy or —NH₂; or N, Z₃ and Z₄ are taken together to form a -(nitrogen-containing 3- to 7-membered monocyclic heterocycle); or N, Z₁ and Z₂ are taken together to form a -(nitrogen-containing 3- to 7-membered monocyclic heterocycle).

5. A method for treating or preventing erectile dysfunction, comprising administering to a subject in need thereof an effective amount of a compound having the Formula (I-152) 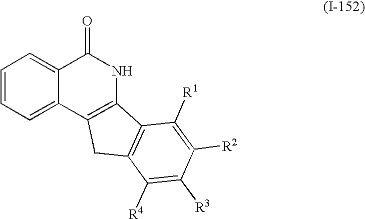
or a pharmaceutically acceptable salt thereof,
wherein
one of the R₁, R², R³ and R⁴ groups is —NH(CH₂)ₙ—N(R⁵)(R⁶) and the remaining groups are simultaneously —H;
R⁵ and R⁶ are independently —H, —C₁-C₆ alkyl or -phenyl, wherein the —C₁-C₆ alkyl or -phenyl is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, -hydroxy or —NH₂; or N, Z₃ and Z₄ are taken together to form an nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C₁-C₁₀ alkyl; or N, R⁵ and R⁶ are taken together to form a nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C₁-C₁₀ alkyl; and
n is an integer ranging from 2 to 6.

6. A method for treating or preventing erectile dysfunction, comprising administering to a subject in need thereof an effective amount of a compound having the Formula (II-152) 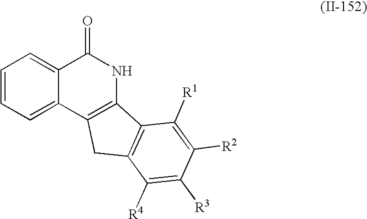
or a pharmaceutically acceptable salt thereof,
wherein
one of the R₁, R², R³ and R⁴ groups is —C(O)NH(CH₂)ₙ—N(R⁵)(R⁶) and the remaining groups are simultaneously —H;
R₅ and R₆ are independently —H, —C₁-C₆ alkyl or -phenyl, wherein the —C₁-C₆ alkyl or -phenyl is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, -hydroxy or —NH₂; or N, Z₃ and Z₄ are taken together to form an nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C₁-C₁₀ alkyl; or N, R⁵ and R⁶ are taken together to form a nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C₁-C₁₀ alkyl; and
n is an integer ranging from 2 to 6.

7. A method for treating or preventing erectile dysfunction, comprising administering to a subject in need thereof an effective amount of a compound having the Formula (I-153) 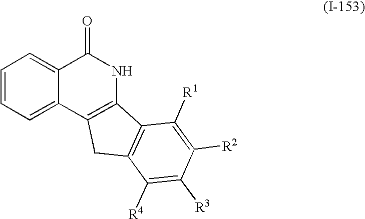
or a pharmaceutically acceptable salt thereof,
wherein
one of the R₁, R², R³ and R⁴ groups is —NHSO₂(CH₂), —N(R⁵)(R⁶) and the remaining groups are simultaneously —H;
R₅ and R₆ are independently —H, —C₁-C₆ alkyl or -phenyl, wherein the —C₁-C₆ alkyl or -phenyl is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, -hydroxy or —NH₂; or N, Z₃ and Z₄ are taken together to form an nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C₁-C₁₀ alkyl; or N, R⁵ and R⁶ are taken together to form a nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C₁-C₁₀ alkyl; and
n is an integer ranging from 1 to 5.

8. A method for treating or preventing erectile dysfunction, comprising administering to a subject in need thereof an effective amount of a compound having the Formula (I-154) 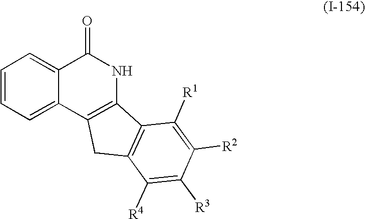
or a pharmaceutically acceptable salt thereof,
wherein
R² and R³ are hydrogen;
one of the R₁ and R⁴ groups is —NHC(O)—(CH₂)ₙ—NR⁵R⁶ and the remaining group is hydrogen;
R₅ and R₆ are independently —H, —C₁-C₆ alkyl or -phenyl, wherein the —C₁-C₆ alkyl or -phenyl is unsubstituted or substituted with one or more of -halo, —OH or —N(Z₃)(Z₄), where Z₃ and Z₄ are independently —H or —C₁-C₅ alkyl, which is unsubstituted or substituted with one or more of -halo, -hydroxy or —NH₂; or N, Z₃ and Z₄ are taken together to form an nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C₁-C₁₀ alkyl; or N, R⁵ and R₆ are taken together to form a nitrogen-containing 3- to 7-membered monocyclic heterocycle which is unsubstituted or substituted with one to three of —C₁-C₅ alkyl, -halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C₁-C₁₀ alkyl; and
n is an integer ranging from 1 to 6.

9. A method for treating or preventing erectile dysfunction, comprising administering to a subject in need thereof an effective amount of a compound having the Formula (II-154) 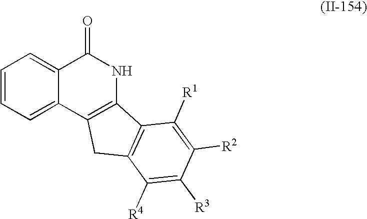
or a pharmaceutically acceptable salt thereof,
wherein
one of the R¹, R², R³, and R⁴ groups is —NHC(O)—(CH₂), —NZ₁Z₂ and the remaining groups are simultaneously hydrogen;
one of Z₁ and Z₂ is —H, —C₁-C₆ alkyl or -phenyl, and the other of Z₁ and Z₂ is -phenyl, wherein the -phenyl in each instance is unsubstituted or substituted with one or more of -halo, —OH or —N(Z₃)(Z₄), where N, Z₃ and Z₄ are taken together to form a nitrogen-containing 3- to 7-membered monocyclic heterocycle which is unsubstituted or substituted with one to three groups of —C₁-C₅ alkyl, -halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C₁-C₁₀ alkyl; or N, Z₁ and Z₂ are taken together to form a nitrogen-containing 3- to 7-membered monocyclic heterocycle, which is substituted with one to three groups of —C₁-C₅ alkyl, -halo, -halo-substituted C₁-C₅ alkyl, hydroxy, —O—C₁-C₅ alkyl, —N(Rₐ)₂, —COOH, —C(O)O—(C₁-C₅ alkyl), —OC(O)—(C₁-C₅ alkyl), —C(O)NH₂, or —NO₂, wherein each occurrence of Rₐ is independently —H, -benzyl, or —C₁-C₁₀ alkyl; and
n is an integer ranging from 1 to 6.

10. A method for treating or preventing erectile dysfunction, comprising administering to a subject in need thereof an effective amount of a compound having the Formula (II-123): 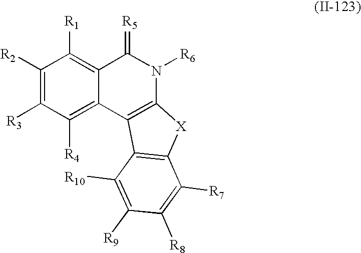
or a pharmaceutically acceptable salt thereof,
wherein:
R₅ is O, NH or S;
R₆ is —H or C₁-C₄ alkyl;
X is —C(O)—, —CH₂—, —CH(halo)-, —(C(OH)((CH₂)ₙCH₃—(C(OH)(aryl))-, —O—, —NH—, —S—, —CH(NR₁₁R₁₂)— or —N(SO₂Y)—, wherein Y is —OH, —NH₂, —C₁-C₅ alkyl)-(3- to 7-membered monocyclic heterocycle), or —(C₁-C₅ alkyl)-(7- to 10-membered bicyclic heterocycle) and n is an integer ranging from 0-5;
R₁₁ and R₁₂ are independently -hydrogen or —C₁-C₉ alkyl, or N, R₁₁ and R₁₂ are taken together to form a -(nitrogen-containing 3- to 7-membered monocyclic heterocycle), or a -(nitrogen-containing 7- to 10-membered bicyclic heterocycle);
R₁, R₂, R₃, R₄, R₇, R₈, R₉ and R₁₀ are independently -hydrogen, -halo, -hydroxy, —O—(C₁-C₅ alkyl), —C₁-C₁₀ alkyl, halo-substituted-(C₁-C₅ alkyl), —C₂-C₁₀ alkenyl, —C₃-C₈-cycloalkyl, -aryl, —NH₂, amino-substituted-(C₁-C₅ alkyl), —C(O)OH, —C(O)O(C₁-C₅ alkyl), —OC(O)(C₁-C₅ alkyl), NO₂ or -A-B;
A is —SO₂—, —SO₂NH—, —NHCO—, —NHCONH—, —O—, —CO—, —OC(O)—, —C(O)O—, —CONH—, —CON(C₁-C₄ alkyl)-, —NH—, —CH₂—, —S— or —C(S)—;
B is —C₁-C₁₀ alkyl, —C₂-C₁₀ alkenyl, -(nitrogen-containing 3- to 7-membered monocyclic heterocycle), -(nitrogen-containing 7- to 10-membered bicyclic heterocycle), -(3- to 7-membered monocyclic heterocycle), -(7- to 10-membered bicyclic heterocycle), —C₃-C₈ cycloalkyl, -aryl, —NZ₁Z₂, —(C₁-C₅ alkylene)-NZ₁Z₂, amino-substituted-(C₁-C₅ alkyl), —N(C₁-C₅ alkyl)(C₁-C₅ alkyl), —(C₁-C₅ alkyl)-(3- to 7-membered monocyclic heterocycle), or —(C₁-C₅ alkyl)-(7- to 10-membered bicyclic heterocycle), —(H₂NC(O))-substituted aryl, —(H₂NC(O))-substituted pyridyl, —C(O)OH, —C(O)O—(C₁-C₅ alkyl), —C(O)O-phenyl or —C(NH)NH₂, each of which is unsubstituted or substituted with one or more of —O—(C₁-C₅ alkyl), -halo, halo-substituted-(C₁-C₅ alkyl), HO-substituted-(C₁-C₅ alkyl), amino-substituted-(C₁-C₅ alkyl), -hydroxy, —NO₂, —NH₂, —CN, —NH(C₁-C₅ alkyl), —N(C₁-C₅ alkyl)(C₁-C₅ alkyl), -(-(nitrogen-containing 3- to 7-membered monocyclic heterocycle)), 7- to 10-membered bicycloheterocyclic amine, —C₁-C₁₀ alkyl, —C₂-C₁₀ alkenyl, —C₂-C₁₀ alkynyl, -aryl, -benzyl, -(H₂NC(O))-substituted(C₁-C₅ alkyl), carboxy-substituted-(C₁-C₅ alkyl), —C(O)OH, —C₁-C₅-alkylene-C(O)O—(C₁-C₅ alkyl) or —C₁-C₅ alkylene-OC(O)—(C₁-C₅ alkyl); and
Z₁ and Z₂ are independently —H or —C₁-C₁₀ alkyl, which is unsubstituted or substituted with one or more of -halo, —OH or —N(Z₃)(Z₄), where Z₃ and Z₄ are independently, —H or —C₁-C₅ alkyl, which is unsubstituted or substituted with one or more of -halo, -hydroxy or —NH₂; or N, Z₃ and Z₄ are taken together to form a -(nitrogen-containing 3- to 7-membered monocyclic heterocycle) or a -(nitrogen-containing 7- to 10-membered bicyclic heterocycle); or N, Z₁ and Z₂ are taken together to form a -(nitrogen-containing 3- to 7-membered monocyclic heterocycle), or a -(nitrogen-containing 7- to 10-membered bicyclic heterocycle).

11. A method for treating or preventing erectile dysfunction, comprising administering to a subject in need thereof an effective amount of a compound having the Formula (IIa-123): 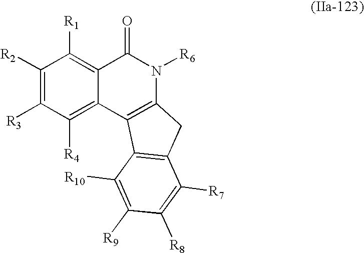
or a pharmaceutically acceptable salt thereof,
wherein:
R₆ is —H or C₁-C₄ alkyl;
R₁, R₂, R₃, R₄, R₇, R₈, R₉ and R₁₀ are independently -hydrogen, -halo, -hydroxy, —O—(C₁-C₅ alkyl), —C₁-C₁₀ alkyl, halo-substituted-(C₁-C₅ alkyl), —C₂-C₁₀ alkenyl, —C₃-C₈-cycloalkyl, -aryl, —NH₂, amino-substituted-(C₁-C₅ alkyl), —C(O)OH, —C(O)O(C₁-C₅ alkyl), —OC(O)(C₁-C₅ alkyl), NO₂ or -A-B;
A is —SO₂—, —SO₂NH—, —NHCO—, —NHCONH—, —O—, —CO—, —OC(O)—, —C(O)O—, —CONH—, —CON(C₁-C₄ alkyl)-, —NH—, —CH₂—, —S— or —C(S)—;
B is —C₁-C₁₀ alkyl, —C₂-C₁₀ alkenyl, -(nitrogen-containing 3- to 7-membered monocyclic heterocycle), -(nitrogen-containing 7- to 10-membered bicyclic heterocycle), -(3- to 7-membered monocyclic heterocycle), -(7- to 10-membered bicyclic heterocycle), —C₃-C₈ cycloalkyl, -aryl, —NZ₁Z₂, —(C₁-C₅ alkylene)-NZ₁Z₂, amino-substituted-(C₁-C₅ alkyl), —N(C₁-C₅ alkyl)(C₁-C₅ alkyl), (C₁-C₅ alkyl)-(3- to 7-membered monocyclic heterocycle), or —(C₁-C₅ alkyl)-(7- to 10-membered bicyclic heterocycle), —(H₂NC(O))-substituted aryl, —(H₂NC(O))-substituted pyridyl, —C(O)OH, —C(O)O—(C₁-C₅ alkyl), —C(O)O-phenyl or —C(NH)NH₂, each of which is unsubstituted or substituted with one or more of —O—(C₁-C₅ alkyl), -halo, halo-substituted-(C₁-C₅ alkyl), HO-substituted-(C₁-C₅ alkyl), amino-substituted-(C₁-C₅ alkyl), -hydroxy, —NO₂, —NH₂, —CN, —NH(C₁-C₅ alkyl), —N(C₁-C₅ alkyl)(C₁-C₅ alkyl), -(-(nitrogen-containing 3- to 7-membered monocyclic heterocycle)), 7- to 10-membered bicycloheterocyclic amine, —C₁-C₁₀ alkyl, —C₂-C₁₀ alkenyl, —C₂-C₁₀ alkynyl, -aryl, -benzyl, —(H₂NC(O))-substituted(C₁-C₅ alkyl), carboxy-substituted-(C₁-C₅ alkyl), —C(O)OH, —C₁-C₅-alkylene-C(O)O—(C₁-C₅ alkyl) or —C₁-C₅ alkylene-OC(O)—(C₁-C₅ alkyl); and
Z₁ and Z₂ are independently —H or —C₁-C₁₀ alkyl, which is unsubstituted or substituted with one or more of -halo, —OH or —N(Z₃)(Z₄), where Z₃ and Z₄ are independently, —H or —C₁-C₅ alkyl, which is unsubstituted or substituted with one or more of -halo, -hydroxy or —NH₂; or N, Z₃ and Z₄ are taken together to form a -(nitrogen-containing 3- to 7-membered monocyclic heterocycle) or a -(nitrogen-containing 7- to 10-membered bicyclic heterocycle); or N, Z₁ and Z₂ are taken together to form a -(nitrogen-containing 3- to 7-membered monocyclic heterocycle), or a -(nitrogen-containing 7- to 10-membered bicyclic heterocycle).

12. A method for treating or preventing erectile dysfunction, comprising administering to a subject in need thereof an effective amount of a compound having the Formula (VI-123): 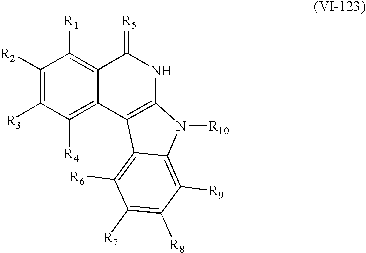
or a pharmaceutically acceptable salt thereof,
wherein:
R₅ is O, S, or NH;
R₁, R₂, R₃, R₄, R₆, R₇, R₈, and R₉ are independently -hydrogen, -halo, -hydroxy, —NH₂ NO₂, or -A-B;
A is —SO₂—, —SO₂NH—, —NHCO—, —NHCONH—, —O—, —CO—, —OC(O)—, —C(O)O—, —CONH—, —CON(C₁-C₄ alkyl)-, —NH—, —CH₂—, —S— or —C(S)—;
B is —C₁-C₁₀ alkyl, —C₂-C₁₀ alkenyl, -(nitrogen-containing 3- to 7-membered monocyclic heterocycle), -(nitrogen-containing 7- to 10-membered bicyclic heterocycle), -(3- to 7-membered monocyclic heterocycle), -(7- to 10-membered bicyclic heterocycle), —C₃-C₈ cycloalkyl, -aryl, —NZ₁Z₂, —(C₁-C₅ alkylene)-NZ₁Z₂, amino-substituted-(C₁-C₅ alkyl), —N(C₁-C₅ alkyl)(C₁-C₅ alkyl), —(C₁-C₅ alkyl)-(3- to 7-membered monocyclic heterocycle), or —(C₁-C₅ alkyl)-(7- to 10-membered bicyclic heterocycle), —(H₂NC(O))-substituted aryl, —(H₂NC(O))-substituted pyridyl, —C(O)OH, —C(O)O—(C₁-C₅ alkyl), —C(O)O-phenyl or —C(NH)NH₂, each of which is unsubstituted or substituted with one or more of —O—(C₁-C₅ alkyl), -halo, halo-substituted-(C₁-C₅ alkyl), HO-substituted-(C₁-C₅ alkyl), amino-substituted-(C₁-C₅ alkyl), -hydroxy, —NO₂, —NH₂, —CN, —NH(C₁-C₅ alkyl), —N(C₁-C₅ alkyl)(C₁-C₅ alkyl), -(nitrogen-containing 3- to 7-membered monocyclic heterocycle), 7- to 10-membered bicycloheterocyclic amine, -C₁-C₁₀ alkyl, —C₂-C₁₀ alkenyl, —C₂-C₁₀ alkynyl, -aryl, -benzyl, —(H₂NC(O))-substituted(C₁-C₅ alkyl), carboxy-substituted-(C₁-C₅ alkyl), —C(O)OH, —C₁-C₅-alkylene-C(O)O—(C₁-C₅ alkyl) or —C₁-C₅ alkylene-OC(O)—(C₁-C₅ alkyl);
Z₁ and Z₂ are independently —H or —C₁-C₁₀ alkyl, which is unsubstituted or substituted with one or more of -halo, —OH or —N(Z₃)(Z₄), where Z₃ and Z₄ are independently, —H or —C₁-C₅ alkyl, which is unsubstituted or substituted with one or more of -halo, -hydroxy or —NH₂; or N, Z₃ and Z₄ are taken together to form a -(nitrogen-containing 3- to 7-membered monocyclic heterocycle) or a -(nitrogen-containing 7- to 10-membered bicyclic heterocycle); or N, Z₁ and Z₂ are taken together to form a -(nitrogen-containing 3- to 7-membered monocyclic heterocycle), or a -(nitrogen-containing 7- to 10-membered bicyclic heterocycle);
R₁₀ is —H, —C₁-C₅ alkyl, —(CH₂)ₙ—CN, —(CH₂)ₙ-aryl, —(CH₂)ₙ-(3- to 7-membered monocyclic heterocycle), —(CH₂)ₙ-7- to 10-membered bicyclic heterocycle), —(CH₂), —COO—(C₁-C₅ alkyl), —(CH₂)ₙ—COO-aryl, —(CH₂)ₙ—COOH, —CONH—(CH₂), —COOH, —CONH—(CH₂)ₙ—COO—(C₁-C₅ alkyl), —CONH—(CH₂)ₙ-aryl, —CONHNH—(C₁-C₅ alkyl), —CONHNH-aryl, —(CH₂)ₙ—CONH₂, —(CH₂), —CONH—(C₁-C₅ alkyl), —(CH₂)ₙ—CONH-aryl, —(CH₂)ₙ—CONH—(CH₂)_{q}-aryl, —(CH₂)ₙ—CONH—(CH₂)_{q}-(3- to 7-membered monocyclic heterocycle), —(CH₂)ₙ—CONH—(CH₂)_{q}-(3- to 7-membered monocyclic heterocycle), —(CH₂)ₙ—CONH—(CH₂)_{q}—CONH₂—(CH₂)ₙ—CONH—(CH₂)_{q}—CONH—(C₁-C₅ alkyl), —(CH₂)ₙ—CONH—(CH₂)_{q}—CON(C₁-C₅ alkyl)₂, —C(O)(CH₂)ₙ—(C₁-C₅ alkyl), —C(O)(CH₂)ₙ-aryl, —C(O)(CH₂)ₙ—COOH, —C(O)(CH₂)ₙ—COO—(C₁-C₅alkyl), —C(O)(CH₂), —COO-(3- to 7-membered monocyclic heterocycle), —C(O)(CH₂), —COO-(7- to 10-membered bicyclic heterocycle), —C(O)(CH₂)ₙ-phenyl, —C(O)(CH₂)ₙ-(3- to 7-membered monocyclic heterocycle), —C(O)(CH₂)ₙ-phenyl, —C(O)(CH₂)ₙ-(7- to 10-membered bicyclic heterocycle), —C(O)O(CH₂)ₙ-phenyl, —C(O)O(CH₂)ₙ-(3- to 7-membered monocyclic heterocycle), —C(O)(CH₂)ₙ-phenyl, —C(O)(CH₂)ₙ-(7- to 10-membered bicyclic heterocycle), —C(O)N((CH₂)ₙ-phenyl)₂, —C(O)N((CH₂)ₙ-phenyl)((CH₂)_{q}-3- to 7-membered monocyclic heterocycle), —C(O)N((CH₂)ₙ-phenyl)((CH₂)_{q 7}- to 10-membered bicyclic heterocycle), —C(O)N((CH₂)ₙ-(3- to 7-membered monocyclic heterocycle)₂, —C(O)N((CH₂)ₙ-7- to 10-membered bicyclic heterocycle)₂, or —SO₂NH₂;
each n is an integer ranging from 0 to 10; and
q is an integer ranging from 0 to 10.
